(19) United States
(12) Patent Application Publication
(10) Pub. No.: US 2025/0066808 A1
(43) Pub. Date: Feb. 27, 2025

(54) NOVEL INSECTICIDAL PROTEINS TOXIC OR INHIBITORY TO HEMIPTERAN PESTS

(71) Applicant: Monsanto Technology LLC, St. Louis, MO (US)

(72) Inventors: David J. Bowen, Wildwood, MO (US); Catherine A. Chay, Ballwin, MO (US); Arlene R. Howe, Clarkson Valley, MO (US); Jennifer L. Lutke, Ballwin, MO (US); Eric Van Fleet, Ballwin, MO (US)

(21) Appl. No.: 18/885,363

(22) Filed: Sep. 13, 2024

Related U.S. Application Data
(62) Division of application No. 17/586,104, filed on Jan. 27, 2022.
Application No. 17/586,104 is a division of application No. 16/805,362, filed on Feb. 28, 2020, now Pat. No. 11,254,950.
Application No. 16/805,362 is a division of application No. 15/627,164, filed on Jun. 19, 2017, now Pat. No. 10,612,037.
(60) Provisional application No. 62/352,136, filed on Jun. 20, 2016.

(51) Int. Cl. C12N 15/82; A01N 37/46; A01N 63/23; C07K 14/32

(52) U.S. Cl. CPC C12N 15/8286; A01N 37/46; A01N 63/23; C07K 14/32; Y02A 40/146

(57) ABSTRACT

Nucleotide sequences are disclosed encoding novel, insecticidal TIC4747 and related proteins exhibiting Hemipteran and Lepidopteran inhibitory activity, as well as fragments thereof. Particular embodiments provide compositions and transformed plants, plant parts, and seeds containing a polynucleotide construct encoding one or more of the toxin proteins within the TIC4747-related protein toxin class.

### CROSS REFERENCE TO RELATED APPLICATION

This application is a divisional of U.S. patent application Ser. No. 17/586,104, filed Jan. 27, 2022, which is a divisional of U.S. patent application Ser. No. 16/805,362, filed Feb. 28, 2020, now U.S. Pat. No. 11,254,950, which is a divisional of U.S. patent application Ser. No. 15/627,164, filed Jun. 19, 2017, now U.S. Pat. No. 10,612,037, which claims the benefit of U.S. Provisional Application No. 62/352,136, filed Jun. 20, 2016, each of which is herein incorporated by reference in its entirety.

### INCORPORATION OF SEQUENCE LISTING

The file named “MONS420USD3_ST26.xml” containing the Sequence Listing was created on Sep. 12, 2024. This file is 107,556 bytes in size (as measured in the MS-Windows® operating system), is filed contemporaneously with this application by electronic submission, and is incorporated herein by reference in its entirety.

### FIELD OF THE INVENTION

The invention generally relates to the field of insect inhibitory proteins. A novel class of insecticidal proteins exhibiting insect inhibitory activity against agriculturally-relevant pests of crop plants and seeds is disclosed in this application. In particular, the disclosed class of proteins exhibits insecticidal activity against agriculturally-relevant pests of crop plants and seeds, particularly the Hemipteran order of insect pests. Plants, plant parts, and seeds containing a recombinant nucleic acid molecule encoding one or more of the disclosed toxin proteins are provided.

### BACKGROUND OF THE INVENTION

Improving crop yield from agriculturally-significant plants including, among others, corn, soybean, sugarcane, rice, wheat, vegetables, and cotton, has become increasingly important. In addition to the growing need for agricultural products to feed, clothe and provide energy for a growing human population, climate-related effects and pressure from the growing population to use land other than for agricultural practices are predicted to reduce the amount of arable land available for farming. These factors have led to grim forecasts with respect to food security, particularly in the absence of major improvements in plant biotechnology and agronomic practices. In light of these pressures, environmentally sustainable improvements in technology, agricultural techniques, and pest management are vital tools to expand crop production on the limited amount of arable land available for farming.

Insects, particularly insects within the order Hemiptera, are considered a major cause of damage to field crops, thereby decreasing crop yields in infested areas. Hemipteran pest species which negatively impact agriculture include, but are not limited to, stink bug, Western tarnished plant bug and Tarnished plant bug.

Historically, the intensive application of synthetic chemical insecticides was relied upon as the pest control agent in agriculture. Concerns for the environment and human health, in addition to emerging resistance issues, stimulated the research and development of biological pesticides. This research effort led to the progressive discovery and use of various entomopathogenic microbial species, including bacteria.

The biological control paradigm shifted when the potential of entomopathogenic bacteria, especially bacteria belonging to the genus Bacillus, was discovered and developed as a biological pest control agent. Strains of the bacterium Bacillus thuringiensis (Bt) have been used as a source for insecticidal proteins since it was discovered that Bt strains show a high toxicity against specific insects. Bt strains are known to produce delta-endotoxins that are localized within parasporal crystalline inclusion bodies at the onset of sporulation and during the stationary growth phase (e.g., Cry proteins), and are also known to produce secreted insecticidal protein. Upon ingestion by a susceptible insect, delta-endotoxins as well as secreted toxins exert their effects at the surface of the midgut epithelium, disrupting the cell membrane, leading to cell disruption and death. Genes encoding insecticidal proteins have also been identified in bacterial species other than Bt, including other Bacillus and a diversity of other bacterial species, such as Brevibacillus laterosporus, Lysinibacillus sphaericus (“Ls” formerly known as Bacillus sphaericus) and Paenibacillus popilliae.

Crystalline and secreted soluble insecticidal protein toxins are highly specific for their hosts and have gained worldwide acceptance as alternatives to chemical insecticides. For example, insecticidal toxin proteins have been employed in various agricultural applications to protect agriculturally important plants from insect infestations, decrease the need for chemical pesticide applications, and increase yields. Insecticidal toxin proteins are used to control agriculturally-relevant pests of crop plants by mechanical methods, such as spraying to disperse microbial formulations containing various bacteria strains onto plant surfaces, and by using genetic transformation techniques to produce transgenic plants and seeds expressing insecticidal toxin protein.

The use of transgenic plants expressing insecticidal proteins has been globally adopted. For example, in 2012, 26.1 million hectares were planted with transgenic crops expressing Bt toxins (James, C., Global Status of Commercialized Biotech/GM Crops: 2012. ISAAA Brief No. 44). The global use of transgenic insect-protected crops and the limited number of insecticidal proteins used in these crops has created a selection pressure for existing insect alleles that impart resistance to the currently-utilized insecticidal proteins.

Hemipteran pests injure plants by puncturing tissues with their piercing and sucking mouthparts and then extracting plant fluids. The principle damage to tissues comes from loss of plant fluids and injection of digestive enzymes. The puncture sites can be predisposed to colonization by pathogenic organisms, causing significant yield and quality loss. One family in the order of Hemiptera, Pentatomidae, has many members commonly referred to as Stink Bugs. Certain species of Stink Bugs that are agricultural pests have become difficult to control with insecticidal chemicals due to the emergence of resistant populations.

Various characteristics of Stink Bugs result in a high susceptibility to resistance emergence. For example, Stink Bugs can have multiple generations in a single growing season. Further, they can overwinter as adults almost anywhere where they can be protected (including homes), and then begin feeding on various host plants as the temperatures rise. Once development begins, Stink Bugs have two to three generations, depending on the species and host plants.

On corn, Stink Bugs are most often found feeding on young plants in late spring and early summer. Feeding at this time results in holes in the leaf, and if severe, results in deformed plants. The Brown Stink Bug is the primary Stink Bug pest for corn crops during the early vegetative stages. The Brown Marmorated Stink Bug, another corn pest, is generally found in late summer on corn usually feeding on the ear, directly destroying the kernels. This is especially a concern on sweet corn and can be quite damaging.

On soybean, most of the injury from Stink Bugs occurs during second half of the growing season when significant feeding takes place on pods and developing seed. Darkish spots will occur where the mouthparts puncture the plant tissue, although these are difficult to see. This can cause deformation and abortion of the seeds, as well as provide a route for infection by pathogenic organisms. Stink Bug feeding on soybean often results in delayed leaf maturity and foliage retention. During seed formation, seeds will become shriveled, deformed, undersized, and even be aborted. Feeding on more developed seeds will result in minor shriveling and discoloration. This not only negatively affects yield, but also will result in lower market value or inhibit the sale of produced seed.

Due to warming temperatures, stink bugs are expanding their population size and geographical range in the United States. Due to the damage on agricultural crops inflicted by stink bugs, their high susceptibility to the emergence there is a need to find new and novel insect toxins that are effective against Hemipteran insect pests such as stink bugs.

### SUMMARY OF THE INVENTION

The present invention provides a novel class of insect inhibitory polypeptides (insect toxic proteins) which are shown to exhibit inhibitory activity against several hemipteran pests of crop plants. Each of the proteins can be used alone or in combination with each other and with other Bt proteins and toxic agents in formulations and in planta, providing alternatives to known Bt proteins and insecticide chemistries.

In one embodiment, a recombinant nucleic acid molecule is disclosed comprising a heterologous promoter fragment operably linked to a polynucleotide segment encoding a pesticidal protein or fragment thereof, wherein said pesticidal protein or pesticidal fragment thereof (a) comprises the amino acid sequence of SEQ ID NO:12, SEQ ID NO:2, SEQ ID NO:4, SEQ ID NO:6, SEQ ID NO:8, SEQ ID NO:10, SEQ ID NO:14, SEQ ID NO:16, SEQ ID NO:18, SEQ ID NO:20, SEQ ID NO:22, SEQ ID NO:24, SEQ ID NO:26, or SEQ ID NO:28; or (b) comprises an amino acid sequence having at least from about 62% to about 100% amino acid sequence identity, or any fraction percentage point between 62% and 100%, to the proteins selected from the group consisting of SEQ ID NO:12, SEQ ID NO:2, SEQ ID NO:4, SEQ ID NO:6, SEQ ID NO:8, SEQ ID NO:10, SEQ ID NO:14, SEQ ID NO:16, SEQ ID NO:18, SEQ ID NO:20, SEQ ID NO:22, SEQ ID NO:24, SEQ ID NO:26, and SEQ ID NO:28. In another embodiment, said polynucleotide segment hybridizes to a polynucleotide having the nucleotide sequence of SEQ ID NO:11, SEQ ID NO:1, SEQ ID NO:3, SEQ ID NO:5, SEQ ID NO:7, SEQ ID NO:9, SEQ ID NO:13, SEQ ID NO:15, SEQ ID NO:17, SEQ ID NO:19, SEQ ID NO:21, SEQ ID NO:23, SEQ ID NO:25, or SEQ ID NO:27 under hybridization conditions of 68° C., followed by washing at 68° C., in 2×SSC containing 0.1% SDS; or (b) said recombinant nucleic acid molecule is in operable linkage with a vector, and said vector is selected from the group consisting of a plasmid, phagemid, bacmid, cosmid, and a bacterial or yeast artificial chromosome. The recombinant nucleic acid molecule can comprise a sequence that functions to express the pesticidal protein in a plant; or is expressed in a plant cell to produce a pesticidally effective amount of pesticidal protein.

In another embodiment, host cells are provided which contain at least one recombinant nucleic acid molecule disclosed herein, wherein the host cell is selected from the group consisting of a bacterial, a yeast, and a plant cell. Bacterial host cells include at least species such as Agrobacterium, Rhizobium, Bacillus, Brevibacillus, Escherichia, Pseudomonas, Klebsiella, and Erwinia. The Bacillus species is a Bacillus cereus or a Bacillus thuringiensis, the Brevibacillus is a Brevibacillus laterosperous, and said Escherichia is an Escherichia coli. Yeast host cells include at least Pichya and Saccharomyces species. Plant host cells include at least dicotyledonous plant cells and monocotyledonous plant cells, and as applicable, further include at least an alfalfa, banana, barley, bean, broccoli, cabbage, brassica, carrot, cassava, castor, cauliflower, celery, chickpea, Chinese cabbage, citrus, coconut, coffee, corn, clover, cotton, a cucurbit, cucumber, Douglas fir, eggplant, eucalyptus, flax, garlic, grape, hops, leek, lettuce, Loblolly pine, millets, melons, nut, oat, olive, onion, ornamental, palm, pasture grass, pea, peanut, pepper, pigeonpea, pine, potato, poplar, pumpkin, Radiata pine, radish, rapeseed, rice, rootstocks, rye, safflower, shrub, sorghum, Southern pine, soybean, spinach, squash, strawberry, sugar beet, sugarcane, sunflower, sweet corn, sweet gum, sweet potato, switchgrass, tea, tobacco, tomato, triticale, turf grass, watermelon, and wheat plant cells.

In another embodiment, the pesticidal protein exhibits activity against an insect species of the order Hemiptera, including Chinavia hilaris, Chinavia marginata, Chinavia pensylvanica, Chlorochroa granulose, Chlorochroa kanei, Chlorochroa ligata, Chlorochroa lineate, Chlorochroa opuntiae, Chlorochroa persimilis, Chlorochroa rossiana, Chlorochroa sayi, Chlorochroa uhleri, Chlorochroa belfragii, Chlorochroa faceta, Chlorochroa osborni, Chlorochroa saucia, Chlorochroa senilis, Nezara viridula, Edessa meditabunda, Edessa bifida, Edessa florida, Euschistus heros, Euschistus acuminatus, Euschistus biformis, Euschistus conspersus, Euschistus crenator, Euschistus egglestoni, Euschistus ictericus, Euschistus inflatus, Euschistus latimarginatus, Euschistus obscures, Euschistus politus, Euschistus quadrator, Euschistus sevus, Euschistus strenuous, Euschistus tristigmus, Euschistus variolarius Halyomorpha halys, Thyanta accerra, Thyanta calceata, Thyanta custator, Thyanta pallidovirens, Thyanta perditor, Thyanta maculate, Thyanta pseudocasta Dichelops melacanthus, Dichelops avilapiresi, Dichelops bicolor, Dichelops dimidatus, Dichelops furcatus, Dichelops furcifrons, Dichelops lobatus, Dichelops miriamae, Dichelops nigrum, Dichelops peruanus, Dichelops phoenix, Dichelops saltensis, Piezodorus guildinni, Piezodorus lituratus Megacopta cribraria, Lygus hesperus, and Lygus lineolaris.

Also provided are plants comprising a recombinant nucleic acid molecule comprising a polynucleotide segment corresponding to a heterologous promoter operably linked to a segment encoding a pesticidal protein or pesticidal fragment thereof, wherein: (a) said pesticidal protein or pesticidal fragment thereof comprises the amino acid sequence of SEQ ID NO:12, SEQ ID NO:2, SEQ ID NO:4, SEQ ID NO:6, SEQ ID NO:8, SEQ ID NO:10, SEQ ID NO:14, SEQ ID NO:16, SEQ ID NO:18, SEQ ID NO:20, SEQ ID NO:22, SEQ ID NO:24, SEQ ID NO:26, or SEQ ID NO:28; or (b) said pesticidal protein or pesticidal fragment thereof comprises an amino acid sequence having at least from about 62% to about 100% amino acid sequence identity, or any fraction percentage point between 62% and 100%, to the proteins selected from the group consisting of SEQ ID NO:12, SEQ ID NO:2, SEQ ID NO:4, SEQ ID NO:6, SEQ ID NO:8, SEQ ID NO:10, SEQ ID NO:14, SEQ ID NO:16, SEQ ID NO:18, SEQ ID NO:20, SEQ ID NO:22, SEQ ID NO:24, SEQ ID NO:26, and SEQ ID NO:28; or (c) said polynucleotide segment hybridizes under stringent hybridization conditions to a polynucleotide having the nucleotide sequence of SEQ ID NO:11, SEQ ID NO:1, SEQ ID NO:3, SEQ ID NO:5, SEQ ID NO:7, SEQ ID NO:9, SEQ ID NO:13, SEQ ID NO:15, SEQ ID NO:17, SEQ ID NO:19, SEQ ID NO:21, SEQ ID NO:23, SEQ ID NO:25, or SEQ ID NO:27 under hybridization conditions of 68° C., followed by washing at 68° C., in 2×SSC containing 0.1% SDS; or (d) said plant exhibits a detectable amount of said pesticidal protein, wherein the pesticidal protein is chosen from the group consisting of SEQ ID NO:12, SEQ ID NO:2, SEQ ID NO:4, SEQ ID NO:6, SEQ ID NO:8, SEQ ID NO:10, SEQ ID NO:14, SEQ ID NO:16, SEQ ID NO:18, SEQ ID NO:20, SEQ ID NO:22, SEQ ID NO:24, SEQ ID NO:26, and SEQ ID NO:28.

In one embodiment, the plant is either a dicotyledonous plant or a monocotyledonous plant. In another embodiment, the plant is further selected from the group consisting of an alfalfa, banana, barley, bean, broccoli, cabbage, brassica, carrot, cassava, castor, cauliflower, celery, chickpea, Chinese cabbage, citrus, coconut, coffee, corn, clover, cotton, a cucurbit, cucumber, Douglas fir, eggplant, eucalyptus, flax, garlic, grape, hops, leek, lettuce, Loblolly pine, millets, melons, nut, oat, olive, onion, ornamental, palm, pasture grass, pea, peanut, pepper, pigeon pea, pine, potato, poplar, pumpkin, Radiata pine, radish, rapeseed, rice, rootstocks, rye, safflower, shrub, sorghum, Southern pine, soybean, spinach, squash, strawberry, sugar beet, sugarcane, sunflower, sweet corn, sweet gum, sweet potato, switchgrass, tea, tobacco, tomato, triticale, turf grass, watermelon, and wheat.

In a further embodiment, seeds comprising the recombinant nucleic acid molecules are disclosed.

In another embodiment, an insect inhibitory composition is provided comprising the recombinant nucleic acid molecules as set forth herein. The insect inhibitory composition can further comprise a nucleotide sequence encoding at least one other pesticidal agent that is different from said pesticidal protein. In certain embodiments, the at least one other pesticidal agent is selected from the group consisting of an insect inhibitory protein, an insect inhibitory dsRNA molecule, and an ancillary protein. The at least one other pesticidal agent in the insect inhibitory composition exhibits activity against one or more pest species of the orders Lepidoptera, Coleoptera, Hemiptera, Homoptera, or Thysanoptera. The at least one other pesticidal agent in the insect inhibitory composition is selected from the group consisting of a Cry1A, Cry1Ab, Cry1Ac, Cry1A.105, Cry1Ae, Cry1B, Cry1C, Cry1C variants, Cry1D, Cry1E, Cry1F, Cry1A/F chimeras, Cry1G, Cry1H, Cry1I, Cry1J, Cry1K, Cry1L, Cry2A, Cry2Ab, Cry2Ae, Cry3, Cry3A variants, Cry3B, Cry4B, Cry6, Cry7, Cry8, Cry9, Cry15, Cry34, Cry35, Cry43A, Cry43B, Cry51Aa1, ET29, ET33, ET34, ET35, ET66, ET70, TIC400, TIC407, TIC417, TIC431, TIC800, TIC807, TIC834, TIC853, TIC900, TIC901, TIC1201, TIC1415, TIC3131, TIC2160, VIP3A, VIP3B, VIP3Ab, AXMI-001, AXMI-002, AXMI-030, AXMI-035, AXMI-036, AXMI-045, Axmi52, Axmi58, Axmi88, Axmi97, Axmi102, Axmi112, Axmi117, Axmi100, AXMI-115, AXMI-113, and AXMI-005, AXMI134, AXMI-150, Axmi171, AXMI-184, axmi196, axmi204, axmi207, axmi209, Axmi205, AXMI218, AXMI220, AXMI221z, AXMI222z, AXMI223z, AXMI224z and AXMI225z, AXMI238, AXMI270, AXMI279, AXMI335, AXMI345, AXMI-R1, and variants thereof, IP3 and variants thereof, DIG-3, DIG-5, DIG-10, DIG-11, DIG-657 protein, PHI-4 variants, PIP-72 variants, PIP-45 variants, PIP-64 varients, PIP-74 variants, PIP-77 variants, DIG-305, PIP-47 varients, DIG-17, DIG-90, DIG-79, and DIG-303.

Commodity products are provided comprising a detectable amount of the recombinant nucleic acid molecules disclosed herein. Such commodity products include commodity corn which may be bagged by a grain handler, corn flakes, corn cakes, corn flour, corn meal, corn syrup, corn oil, corn silage, corn starch, corn cereal, and the like, and corresponding soybean, rice, wheat, sorghum, pigeon pea, peanut, fruit, melon, and vegetable commodity products including, where applicable, juices, concentrates, jams, jellies, marmalades, and other edible forms of such commodity products containing a detectable amount of such polynucleotides and or polypeptides of this application, whole or processed cotton seed, cotton oil, lint, seeds and plant parts processed for feed or food, fiber, paper, biomasses, and fuel products such as fuel derived from cotton oil or pellets derived from cotton gin waste, whole or processed soybean seed, soybean oil, soybean protein, soybean meal, soybean flour, soybean flakes, soybean bran, soybean milk, soybean cheese, soybean wine, animal feed comprising soybean, paper comprising soybean, cream comprising soybean, soybean biomass, and fuel products produced using soybean plants and soybean plant parts.

Also contemplated is a method of producing seed comprising one or more of the recombinant nucleic acid molecules disclosed herein. The method includes planting at least one such seed; growing a plant from the seed; and harvesting progeny seed from the plant, wherein the harvested seed comprises the one or more recombinant nucleic acid molecules.

In another embodiment, a plant resistant to insect infestation is provided. The cells of said plant optionally comprise: (a) a recombinant nucleic acid molecule encoding an insecticidally effective amount of a pesticidal protein as set forth in SEQ ID NO:12, SEQ ID NO:2, SEQ ID NO:4, SEQ ID NO:6, SEQ ID NO:8, SEQ ID NO:10, SEQ ID NO:14, SEQ ID NO:16, SEQ ID NO:18, SEQ ID NO:20, SEQ ID NO:22, SEQ ID NO:24, SEQ ID NO:26, or SEQ ID NO:28; or (b) an insecticidally effective amount of a protein comprising an amino acid sequence having at least 62%, or 65%, or 70%, or 80%, or 85%, or 90%, or 95%, or about 100% amino acid sequence identity to SEQ ID NO:12, SEQ ID NO:2, SEQ ID NO:4, SEQ ID NO:6, SEQ ID NO:8, SEQ ID NO:10, SEQ ID NO:14, SEQ ID NO:16, SEQ ID NO:18, SEQ ID NO:20, SEQ ID NO:22, SEQ ID NO:24, SEQ ID NO:26, or SEQ ID NO:28.

Also disclosed are methods for controlling a Lepidopteran species pest, and controlling a Hemipteran species pest infestation of a plant, particularly a crop plant. The method will comprise contacting the pest with an insecticidally effective amount of a pesticidal proteins as set forth in SEQ ID NO:12, SEQ ID NO:2, SEQ ID NO:4, SEQ ID NO:6, SEQ ID NO:8, SEQ ID NO:10, SEQ ID NO:14, SEQ ID NO:16, SEQ ID NO:18, SEQ ID NO:20, SEQ ID NO:22, SEQ ID NO:24, SEQ ID NO:26, or SEQ ID NO:28; or contacting the pest with an insecticidally effective amount of one or more pesticidal proteins comprising an amino acid sequence having at least 62%, or 65%, or 70%, or 80%, or 85%, or 90%, or 95%, or about 100% amino acid sequence identity to SEQ ID NO:12, SEQ ID NO:2, SEQ ID NO:4, SEQ ID NO:6, SEQ ID NO:8, SEQ ID NO:10, SEQ ID NO:14, SEQ ID NO:16, SEQ ID NO:18, SEQ ID NO:20, SEQ ID NO:22, SEQ ID NO:24, SEQ ID NO:26, or SEQ ID NO:28.

### BRIEF DESCRIPTION OF THE SEQUENCES

SEQ ID NO:1 is the nucleotide sequence encoding the TIC4747 toxin protein obtained from Bacillus thuringiensis (Bt) species CFB007452.

SEQ ID NO:2 is the amino acid sequence of TIC4747 protein.

SEQ ID NO:3 is the nucleotide sequence encoding the TIC7181 toxin protein obtained from Bt species EG9737.

SEQ ID NO:4 is the amino acid sequence of TIC7181 protein.

SEQ ID NO:5 is the nucleotide sequence encoding the TIC4904 toxin protein obtained from Bt species CFB007432.

SEQ ID NO:6 is the amino acid sequence of TIC4904 protein.

SEQ ID NO:7 is the nucleotide sequence encoding the TIC6547 toxin protein obtained from Bt species CFB231019.

SEQ ID NO:8 is the amino acid sequence of TIC6547 protein.

SEQ ID NO:9 is the native nucleotide sequence encoding the TIC4006 toxin protein obtained from Bt species WC12466.

SEQ ID NO:10 is the amino acid sequence of TIC4006 protein.

SEQ ID NO:11 is a synthetic coding sequence encoding a TIC4747PL pesticidal protein designed for expression in a plant cell wherein an additional alanine codon is inserted immediately following the initiating methionine codon.

SEQ ID NO:12 is the amino acid sequence of TIC4747PL encoded by a synthetic coding sequence designed for expression in a plant cell (SEQ ID NO:11), and wherein an additional alanine amino acid is inserted immediately following the initiating methionine.

SEQ ID NO:13 is a synthetic coding sequence encoding a TIC7181PL pesticidal protein designed for expression in a plant cell wherein an additional alanine codon is inserted immediately following the initiating methionine codon.

SEQ ID NO:14 is the amino acid sequence of TIC7181PL encoded by a synthetic coding sequence designed for expression in a plant cell (SEQ ID NO:13), and wherein an additional alanine amino acid is inserted immediately following the initiating methionine.

SEQ ID NO:15 is a synthetic coding sequence encoding a TIC4904PL pesticidal protein designed for expression in a plant cell wherein an additional alanine codon is inserted immediately following the initiating methionine codon.

SEQ ID NO:16 is the amino acid sequence of TIC4904PL encoded by a synthetic coding sequence designed for expression in a plant cell (SEQ ID NO:15), and wherein an additional alanine amino acid is inserted immediately following the initiating methionine.

SEQ ID NO:17 is a synthetic coding sequence encoding a TIC6547PL pesticidal protein designed for expression in a plant cell wherein an additional alanine codon is inserted immediately following the initiating methionine codon.

SEQ ID NO:18 is the amino acid sequence of TIC6547PL encoded by a synthetic coding sequence designed for expression in a plant cell (SEQ ID NO:17), and wherein an additional alanine amino acid is inserted immediately following the initiating methionine.

SEQ ID NO:19 is a synthetic coding sequence encoding a TIC4006PL pesticidal protein designed for expression in a plant cell wherein an additional alanine codon is inserted immediately following the initiating methionine codon.

SEQ ID NO:20 is the amino acid sequence of TIC4006PL encoded by a synthetic coding sequence designed for expression in a plant cell (SEQ ID NO:19), and wherein an additional alanine amino acid is inserted immediately following the initiating methionine.

SEQ ID NO:21 is a nucleotide sequence encoding TIC4747_His which is comprised of the TIC4747 coding sequence with a Histadine tag coding sequence operably linked 3′ to the TIC4747 coding sequence.

SEQ ID NO:22 is the amino acid sequence of TIC4747_His.

SEQ ID NO:23 is a nucleotide sequence encoding TIC4904_His which is comprised of the TIC4904 coding sequence with a Histadine tag coding sequence operably linked 5′ to the TIC4904 coding sequence.

SEQ ID NO:24 is the amino acid sequence of TIC4904_His.

SEQ ID NO:25 is a nucleotide sequence encoding TIC6547_His which is comprised of the TIC6547 coding sequence with a Histadine tag coding sequence operably linked 5′ to the TIC6547 coding sequence.

SEQ ID NO:26 is the amino acid sequence of TIC6547_His.

SEQ ID NO:27 is a nucleotide sequence encoding TIC4006_His which is comprised of the TIC4006 coding sequence with a Histadine tag coding sequence operably linked 5′ to the TIC4006 coding sequence.

SEQ ID NO:28 is the amino acid sequence of TIC4006_His.

### DETAILED DESCRIPTION OF THE INVENTION

One problem in the art of agricultural pest control can be characterized as a need for new insecticidal proteins that are efficacious against target pests, exhibit broad spectrum toxicity against target pest species, are capable of being expressed in plants without causing undesirable agronomic issues, and provide an alternative mode of action compared to current toxins that are used commercially in plants.

Novel insecticidal proteins are disclosed herein, exemplified by TIC4747 and related family members that provide resistance to Hemipteran insect pests. Also disclosed are synthetic coding sequences designed for expression in a plant cell that encode a TIC4747PL and related family member toxin proteins in which an alanine amino acid residue is provided immediately following the initiating methionine residue to improve expression in the plant cell. Further disclosed are recombinant nucleic acid molecules comprising a promoter in operable linkage to a coding sequence encoding a TIC4747 or TIC4747PL toxin protein, or related family members, or fragment thereof.

Reference in this application to “TIC4747 proteins,” “TIC4747 protein toxins,” “TIC4747 toxin proteins,” “TIC4747 pesticidal proteins,” “TIC4747-related toxins,” “TIC4747-related family members,” “TIC4747-related protein toxin class or family,” “TIC4747-related toxin proteins,” “TIC4747-related toxin polypeptides,” “TIC4747-related pesticidal proteins,” “TIC4747 protein toxin class,” “variants of TIC4747,” or “TIC4747 variants” and the like, refer to any novel insect inhibitory protein that comprises, that consists, that is substantially homologous to, that is similar to, or that is derived from the insect inhibitory polypeptide sequence of TIC4747 (SEQ ID NO:2), the insect inhibitory polypeptide sequence of TIC4747-related family members TIC7181 (SEQ ID NO:4), TIC4904 (SEQ ID NO:6), TIC6547 (SEQ ID NO:8) and TIC4006 (SEQ ID NO:10), plant-optimized polypeptides sequences of TIC474 or TIC4747-related family members, and insect inhibitory segments thereof, or combinations thereof, that confer activity against Hemipteran pests, including any protein exhibiting insect inhibitory activity if alignment of such protein with TIC4747 or TIC4747-related family members results in at least about 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or about 100% amino acid sequence identity (or any fraction percentage in this range).

The term “segment” or “fragment” is used in this application to describe consecutive amino acid or nucleic acid sequences that are shorter than the complete amino acid or nucleic acid sequence describing a disclosed TIC4747, TIC4747PL or related family member insecticidal protein. A segment or fragment exhibiting insect inhibitory activity is also disclosed in this application if alignment of such segment or fragment, with the corresponding section of the TIC4747 or TIC4747PL and related family member insecticidal protein, results in amino acid sequence identity of any fraction percentage from about 62 to about 100 percent between the segment or fragment and the corresponding section of the TIC4747 or TIC4747PL and related family member insecticidal protein.

Reference in this application to the terms “active” or “activity”, “pesticidal activity” or “pesticidal”, or “insecticidal activity”, “insect inhibitory” or “insecticidal” refer to efficacy of a toxic agent, such as an insecticidal protein, in inhibiting (inhibiting growth, feeding, fecundity, or viability), suppressing (suppressing growth, feeding, fecundity, or viability), controlling (controlling the pest infestation, controlling the pest feeding activities on a particular crop containing an effective amount of the insecticidal protein) or killing (causing the morbidity, mortality, or reduced fecundity of) a pest. These terms are intended to include the result of providing a pesticidally effective amount of an insecticidal protein to a pest where the exposure of the pest to the insecticidal protein results in morbidity, mortality, reduced fecundity, or stunting. These terms also include repulsion of the pest from the plant, a tissue of the plant, a plant part, seed, plant cells, or from the particular geographic location where the plant may be growing, as a result of providing a pesticidally effective amount of the insecticidal protein in or on the plant. In general, pesticidal activity refers to the ability of an insecticidal protein to be effective in inhibiting the growth, development, viability, feeding behavior, mating behavior, fecundity, or any measurable decrease in the adverse effects caused by an insect feeding on this protein, protein fragment, protein segment or polynucleotide of a particular target pest, including but not limited to insects of the order Hemiptera. The insecticidal protein can be produced by the plant or can be applied to the plant or to the environment within the location where the plant is located. The terms “bioactivity,” “effective,” “efficacious” or variations thereof are also terms interchangeably utilized in this application to describe the effects of the insecticidal proteins of the present invention on target insect pests.

A pesticidally effective amount of a toxic agent, when provided in the diet of a target pest, exhibits pesticidal activity when the toxic agent contacts the pest. A toxic agent can be an insecticidal protein or one or more chemical agents known in the art. Insecticidal chemical agents and insecticidal protein agents can be used alone or in combinations with each other. Chemical agents include but are not limited to dsRNA molecules targeting specific genes for suppression in a target pest, organochlorides, organophosphates, carbamates, pyrethroids, neonicotinoids, and ryanoids. Insecticidal protein agents include the insecticidal proteins set forth in this application, as well as other proteinaceous toxic agents including those that target Hemipteran pest species, as well as protein toxins that are used to control other plant pests such as Cry proteins available in the art for use in controlling Lepidopteran, Coleopteran, Thysanopteranm and Homopteran species.

It is intended that reference to a pest, particularly a pest of a crop plant, means insect pests of crop plants, particularly those Hemipteran insect pests that are controlled by the TIC4747 toxin protein class. However, reference to a pest can also include Lepidopteran, Coleopteran, Homopteran, and Thysanopteran insect pests of plants, as well as nematodes and fungi when toxic agents targeting these pests are co-localized or present together with a TIC4747-related insecticidal protein, or a protein that is 62 to about 100 percent identical to the insecticidal protein.

The insecticidal proteins of the TIC4747 protein toxin class are related by a common function and exhibit insecticidal activity towards insect pests from the Hemipteran insect species as well as the Lepidopteran insect species.

The insects of the order Hemiptera include, but are not limited to, Stink Bugs of the family Pentatomidae: Green Stink Bugs from the genus Chinavia (Chinavia hilaris, Chinavia marginata, and Chinavia pensylvanica), Stink bugs of the genus Chlorochroa (Chlorochroa granulose, Chlorochroa kanei, Chlorochroa ligata, Chlorochroa lineate, Chlorochroa opuntiae, Chlorochroa persimilis, Chlorochroa rossiana, Chlorochroa sayi, Chlorochroa uhleri, Chlorochroa belfragii, Chlorochroa faceta, Chlorochroa osborni, Chlorochroa saucia, and Chlorochroa senilis), Southern Green Stink Bug (Nezara viridula), Stink Bugs from the genus Edessa (Edessa meditabunda, Edessa bifida, and Edessa florida), the Neotropical Brown Stink Bug (Euschistus heros), stink bugs from the genus Euschistus (Euschistus acuminatus, Euschistus biformis, Euschistus conspersus, Euschistus crenator, Euschistus egglestoni, Euschistus ictericus, Euschistus inflatus, Euschistus latimarginatus, Euschistus obscures, Euschistus politus, Euschistus quadrator, Euschistus sevus, Euschistus strenuous, Euschistus tristigmus, and Euschistus variolarius), Brown Marmorated Stink Bug (Halyomorpha halys), Red-Shouldered Stink Bug (Thyanta accerra), stink bugs of the genus Thyanta (Thyanta calceata, Thyanta custator, Thyanta pallidovirens, Thyanta perditor, Thyanta maculate, and Thyanta pseudocasta), the Green Belly Stink Bug (Dichelops melacanthus) and other stink bugs of the genus Dichelops (Dichelops avilapiresi, Dichelops bicolor, Dichelops dimidatus, Dichelops furcatus, Dichelops furcifrons, Dichelops lobatus, Dichelops miriamae, Dichelops nigrum, Dichelops peruanus, Dichelops phoenix, and Dichelops saltensis), the Red Banded Stink Bug (Piezodorus guildinni) as well as Piezodorus lituratus; and insects of the family of Plataspidae such as Kudzu Bug (Megacopta cribraria), Western tarnished plant bug (Lygus hesperus), and Tarnished plant bug (Lygus lineolaris).

The insects of the order Lepidoptera include, but are not limited to, armyworms, cutworms, loopers, and heliothines in the Family Noctuidae, e.g., fall armyworm (Spodoptera frugiperda), beet armyworm (Spodoptera exigua), bertha armyworm (Mamestra configurata), black cutworm (Agrotis ipsilon), cabbage looper (Trichoplusia ni), soybean looper (Pseudoplusia includens), velvetbean caterpillar (Anticarsia gemmatalis), green cloverworm (Hypena scabra), tobacco budworm (Heliothis virescens), granulate cutworm (Agrotis subterranea), armyworm (Pseudaletia unipuncta), western cutworm (Agrotis orthogonia); borers, casebearers, webworms, coneworms, cabbageworms and skeletonizers from the Family Pyralidae, e.g., European corn borer (Ostrinia nubilalis), navel orangeworm (Amyelois transitella), corn root webworm (Crambus caliginosellus), sod webworm (Herpetogramma licarsisalis), sunflower moth (Homoeosoma electellum), lesser cornstalk borer (Elasmopalpus lignosellus); leafrollers, budworms, seed worms, and fruit worms in the Family Tortricidae, e.g., codling moth (Cydia pomonella), grape berry moth (Endopiza viteana), oriental fruit moth (Grapholita molesta), sunflower bud moth (Suleima helianthana); and many other economically important Lepidoptera, e.g., diamondback moth (Plutella xylostella), pink bollworm (Pectinophora gossypiella) and gypsy moth (Lymantria dispar). Other insect pests of order Lepidoptera include, e.g., cotton leaf worm (Alabama argillacea), fruit tree leaf roller (Archips argyrospila), European leafroller (Archips rosana) and other Archips species, Asiatic rice borer or rice stem borer (Chilo suppressalis), rice leaf roller (Cnaphalocrocis medinalis), corn root webworm (Crambus caliginosellus), bluegrass webworm (Crambus teterrellus), southwestern corn borer (Diatraea grandiosella), sugarcane borer (Diatraea saccharalis), spiny bollworm (Earias insulana), spotted bollworm (Earias vittella), American bollworm (Helicoverpa armigera), corn earworm or cotton bollworm (Helicoverpa zea), sod webworm (Herpetogramma licarsisalis), European grape vine moth (Lobesia botrana), citrus leafminer (Phyllocnistis citrella), large white butterfly (Pieris brassicae), imported cabbageworm, or small white butterfly (Pieris rapae), tobacco cutworm or cluster caterpillar (Spodoptera litura), and tomato leafminer (Tuta absoluta).

Reference in this application to an “isolated DNA molecule,” or an equivalent term or phrase, is intended to mean that the DNA molecule is one that is present alone or in combination with other compositions, but not within its natural environment. For example, nucleic acid elements such as a coding sequence, intron sequence, untranslated leader sequence, promoter sequence, transcriptional termination sequence, and the like, that are naturally found within the DNA of the genome of an organism are not considered to be “isolated” so long as the element is within the genome of the organism and at the location within the genome in which it is naturally found. However, each of these elements, and subparts of these elements, would be “isolated” within the scope of this disclosure so long as the element is not within the genome of the organism and at the location within the genome in which it is naturally found. Similarly, a nucleotide sequence encoding an insecticidal protein or any naturally occurring insecticidal variant of that protein would be an isolated nucleotide sequence so long as the nucleotide sequence was not within the DNA of the bacterium from which the sequence encoding the protein is naturally found. A synthetic nucleotide sequence encoding the amino acid sequence of the naturally occurring insecticidal protein would be considered to be isolated for the purposes of this disclosure. For the purposes of this disclosure, any transgenic nucleotide sequence, i.e., the nucleotide sequence of the DNA inserted into the genome of the cells of a plant or bacterium, or present in an extrachromosomal vector, would be considered to be an isolated nucleotide sequence whether it is present within the plasmid or similar structure used to transform the cells, within the genome of the plant or bacterium, or present in detectable amounts in tissues, progeny, biological samples or commodity products derived from the plant or bacterium.

As described further herein, an open reading frame (ORF) encoding TIC4747 (SEQ ID NO:1) was discovered in DNA obtained from Bacillus thuringiensis (Bt) species CFB007452. The coding sequence was cloned and expressed in microbial host cells to produce recombinant proteins used in bioassays. High throughput sequencing and bioinformatic techniques were used to screen microbial genomes for genes encoding proteins exhibiting similarity to TIC4747. The resulting proteins—TIC4904, TIC4006, TIC6547, and TIC7181—along with TIC4747, the polypeptides encoding these proteins, and the plant-optimized polypeptides and polynucleotides for these proteins constitute the TIC4747 protein toxin class. Bioassays using microbial host cell-derived proteins of TIC4747 demonstrated activity against the Hemipteran insect pest species Southern Green Stink Bug (Nezara viridula), Neotropical Brown Stink Bug (Euschistus heros), Western tarnished plant bug (Lygus hesperus), and Tarnished plant bug (Lygus lineolaris). The novel insecticidal protein, TIC6547 exhibits insecticidal activity against the Lepidopteran insect pest species Diamondback Moth (Plutella xylostella). The novel insecticidal protein, TIC7181 demonstrated activity against Tarnished plant bug (Lygus lineolaris).

It is contemplated that additional toxin protein sequences related to TIC4747 can be created by using the amino acid sequence of the proteins in the TIC4747 protein toxin class to create proteins with novel properties. Proteins from the TIC4747 protein toxin class can be aligned to combine differences at the amino acid level into novel amino acid sequence variants and making appropriate changes to the recombinant nucleic acid sequence encoding the variants.

It is further contemplated that improved variants of the TIC4747 toxin class can be engineered in planta by using various gene editing methods known in the art. Such technologies used for genome editing include, but are not limited to, ZFN (zinc-finger nuclease), meganucleases, TALEN (Transcription activator-like effector nucleases), and CRISPR (Clustered Regularly Interspaced Short Palindromic Repeats)/Cas (CRISPR-associated) systems. These genome editing methods can be used to alter the toxin protein coding sequence transformed within a plant cell to a different toxin coding sequence. Specifically, through these methods, one or more codons within the toxin coding sequence is altered to engineer a new protein amino acid sequence. Alternatively, a fragment within the coding sequence is replaced or deleted, or additional DNA fragments are inserted into the coding sequence, to engineer a new toxin coding sequence. The new coding sequence can encode a toxin protein with new properties such as increased activity or spectrum against insect pests, as well as provide activity against an insect pest species wherein resistance has developed against the original insect toxin protein. The plant cell comprising the gene edited toxin coding sequence can be used by methods known in the art to generate whole plants expressing the new toxin protein.

It is also contemplated that fragments of proteins in the TIC4747 protein toxin class can be truncated forms wherein one or more amino acids are deleted from the N-terminal end, C-terminal end, the middle of the protein or combinations thereof wherein the fragments and variants retain insect inhibitory activity. These fragments can be naturally occurring or synthetic variants of proteins in the TIC4747 protein toxin class or derived protein variants, but should retain the insect inhibitory activity of proteins in the TIC4747 protein toxin class.

Proteins that resemble TIC4747 and related family member insecticidal proteins can be identified by comparison to each other using various computer based algorithms known in the art (See Tables 1 and 2). For example, amino acid sequence identities of proteins related to the TIC4747 and related family member insecticidal proteins can be analyzed using a Clustal W alignment using these default parameters: Weight matrix: blosum, Gap opening penalty: 10.0, Gap extension penalty: 0.05, Hydrophilic gaps: On, Hydrophilic residues: GPSNDQERK, Residue-specific gap penalties: On (Thompson, et al (1994) Nucleic Acids Research, 22:4673-4680). Percent amino acid identity is further calculated by the product of 100% multiplied by (amino acid identities/length of the subject protein). Other alignment algorithms are also available in the art, provide results similar to those obtained using Clustal W alignment and are contemplated in this application.

It is intended that a query protein exhibiting insect inhibitory activity against a Hemipteran or Lepidopteran insect species is related to the TIC4747 protein toxin class if alignment of such query protein with the subject TIC4747 and related family member insecticidal proteins set forth in SEQ ID NOs:2, 4, 6, 8, 10, 12, 14, 16, 18, or 20 results in at least about 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or about 100% amino acid sequence identity (or any fraction percentage in this range) between the query and subject protein.

Exemplary proteins of TIC4747 (SEQ ID NO:2) and related family members TIC7181 (SEQ ID NO:4), TIC4904 (SEQ ID NO:6), TIC6547 (SEQ ID NO:8), and TIC4006 (SEQ ID NO: 10) were aligned with each other using a Clustal W algorithm. A pair-wise matrix of percent amino acid sequence identities for each pair was created, as reported in Table 1.

TABLE 1
Pair-wise matrix display of exemplary proteins.

| | TIC4747 | TIC7181 | TIC4904 | TIC6547 | TIC4006 |
|---|---|---|---|---|---|
| | SEQ ID | SEQ ID | SEQ ID | SEQ ID | SEQ ID |
| Sequence | NO: 2 | NO: 4 | NO: 8 | NO: 10 | NO: 12 |
| TIC4747 | — | 99.9 | 94.2 | 93.1 | 82.3 |
| | | (1216) | (1147) | (1133) | (1001) |
| TIC7181 | 99.9 | — | 94.2 | 93 | 82.2 |
| | (1216) | | (1146) | (1132) | (1000) |
| TIC4904 | 94.2 | 94.2 | — | 93.3 | 81.8 |
| | (1147) | (1146) | | (1135) | (995) |
| TIC6547 | 93.6 | 93.5 | 93.7 | — | 82.8 |
| | (1133) | (1132) | (1135) | | (1003) |
| TIC4006 | 82.5 | 82.4 | 82 | 82.7 | — |
| | (1001) | (1000) | (995) | (1003) | |

In addition to percent identity, the TIC4747 and related family member proteins can also be related by primary structure (conserved amino acid motifs), by lengths (about 1211 to 1217 amino acids) and by other characteristics. Characteristics of TIC4747 and related family members are presented in Table 2 below.

TABLE 2
Characteristics of TIC4747 and related family members.

| | | | | | No. of | No. of | No. of | |
|---|---|---|---|---|---|---|---|---|
| | Molecular Weight | Amino Acid | Isoelectric | Charge at | Strongly Basic (−) | Strongly Acidic | Hydrophobic | No. of Polar |
| Protein | (in Daltons) | Length | Point | PH 7.0 | Amino Acids | Amino Acids | Amino Acids | Amino Acids |
| TIC4747 | 137364.49 | 1217 | 6.2857 | −5.0 | 145 | 134 | 601 | 616 |
| TIC7181 | 137378.52 | 1217 | 6.2857 | −5.0 | 145 | 134 | 601 | 616 |
| TIC4904 | 137335.54 | 1217 | 6.1028 | −7.0 | 141 | 134 | 602 | 615 |
| TIC6547 | 136629.45 | 1211 | 6.0733 | −7.0 | 141 | 135 | 597 | 614 |
| TIC4006 | 136704.56 | 1213 | 5.8876 | −9.0 | 135 | 132 | 592 | 621 |

As described further in the Examples of this application, a recombinant nucleic acid molecule sequences encoding the proteins of the TIC4747 protein toxin class were designed for use in plants. One exemplary plant-optimized recombinant nucleic acid molecule sequence encoding TIC4747 is TIC4747PL (SEQ ID NO:11). TIC4747PL has an additional alanine amino acid immediately following the initiating methionine relative to TIC4747. The additional alanine residue inserted into TIC4747 is believed to improve the expression of the protein in planta.

Expression cassettes and vectors containing the recombinant nucleic acid molecule sequence can be constructed and introduced into corn, soybean or cotton plant cells in accordance with transformation methods and techniques known in the art. For example, Agrobacterium-mediated transformation is described in U.S. Patent Application Publications 2009/0138985A1 (soybean), 2008/0280361A1 (soybean), 2009/0142837A1 (corn), 2008/0282432 (cotton), 2008/0256667 (cotton), 2003/0110531 (wheat), 2001/0042257 A1 (sugar beet), U.S. Pat. No. 5,750,871 (canola), U.S. Pat. No. 7,026,528 (wheat), and U.S. Pat. No. 6,365,807 (rice), and in Arencibia et al. (1998) Transgenic Res. 7:213-222 (sugarcane) all of which are incorporated herein by reference in their entirety. Transformed cells can be regenerated into transformed plants that express proteins from the TIC4747 protein toxin class and demonstrate pesticidal activity through bioassays performed in the presence of Lepidopteran or Hemipteran pest larvae using plant leaf disks obtained from the transformed plants.

As an alternative to traditional transformation methods, a DNA sequence, such as a transgene, expression cassette(s), etc., may be inserted or integrated into a specific site or locus within the genome of a plant or plant cell via site-directed integration. Recombinant DNA construct(s) and molecule(s) of this disclosure may thus include a donor template sequence comprising at least one transgene, expression cassette, or other DNA sequence for insertion into the genome of the plant or plant cell. Such donor template for site-directed integration may further include one or two homology arms flanking an insertion sequence (i.e., the sequence, transgene, cassette, etc., to be inserted into the plant genome). The recombinant DNA construct(s) of this disclosure may further comprise an expression cassette(s) encoding a site-specific nuclease and/or any associated protein(s) to carry out site-directed integration. These nuclease expressing cassette(s) may be present in the same molecule or vector as the donor template (in cis) or on a separate molecule or vector (in trans). Several methods for site-directed integration are known in the art involving different proteins (or complexes of proteins and/or guide RNA) that cut the genomic DNA to produce a double strand break (DSB) or nick at a desired genomic site or locus.

As understood in the art, during the process of repairing the DSB or nick introduced by the nuclease enzyme, the donor template DNA may become integrated into the genome at the site of the DSB or nick. The presence of the homology arm(s) in the donor template may promote the adoption and targeting of the insertion sequence into the plant genome during the repair process through homologous recombination, although an insertion event may occur through non-homologous end joining (NHEJ). Examples of site-specific nucleases that may be used include zinc-finger nucleases, engineered or native meganucleases, TALE-endonucleases, and RNA-guided endonucleases (e.g., Cas9 or Cpf1). For methods using RNA-guided site-specific nucleases (e.g., Cas9 or Cpf1), the recombinant DNA construct(s) will also comprise a sequence encoding one or more guide RNAs to direct the nuclease to the desired site within the plant genome.

Recombinant nucleic acid molecule compositions that encode TIC4747 or related family member insecticidal proteins are contemplated. For example, TIC4747 and related family member insecticidal proteins can be expressed with recombinant DNA constructs in which a polynucleotide molecule with an ORF encoding the protein is operably linked to genetic expression elements such as a promoter and any other regulatory element necessary for expression in the system for which the construct is intended. Non-limiting examples include a plant-functional promoter operably linked to TIC4747 or related family member insecticidal protein encoding sequences for expression of the protein in plants or a Bt-functional promoter operably linked to a TIC4747 or related family member protein encoding sequence for expression of the protein in a Bt bacterium or other Bacillus species. Other elements can be operably linked to TIC4747 or related family member protein encoding sequences including, but not limited to, enhancers, introns, untranslated leaders, encoded protein immobilization tags (HIS-tag), translocation peptides (i.e., plastid transit peptides, signal peptides), polypeptide sequences for post-translational modifying enzymes, ribosomal binding sites, and RNAi target sites. Exemplary recombinant polynucleotide molecules provided herewith include, but are not limited to, a heterologous promoter operably linked to a polynucleotide such as SEQ ID NOs:1, 3, 5, 7, 9, 11, 13, 15, 17, and 19 that encodes the polypeptides or proteins having the amino acid sequences set forth in SEQ ID NOs:2, 4, 6, 8, 10, 12, 14, 16, 18, and 20. The codons of a recombinant polynucleotide molecule encoding for proteins disclosed herein can be substituted by synonymous codons (known in the art as a silent substitution).

A recombinant DNA construct comprising TIC4747 or related family member protein encoding sequences can further comprise a region of DNA that encodes for one or more insect inhibitory agents which can be configured to concomitantly express or co-express with a DNA sequence encoding a TIC4747 or related family member protein, a protein different from TIC4747 or related family member proteins, an insect inhibitory dsRNA molecule, or an ancillary protein. Ancillary proteins include, but are not limited to, co-factors, enzymes, binding-partners, or other agents that function to aid in the effectiveness of an insect inhibitory agent, for example, by aiding its expression, influencing its stability in plants, optimizing free energy for oligomerization, augmenting its toxicity, and increasing its spectrum of activity. An ancillary protein may facilitate the uptake of one or more insect inhibitory agents, for example, or potentiate the toxic effects of a toxic agent.

A recombinant DNA construct can be assembled so that all proteins or dsRNA molecules are expressed from one promoter or each protein or dsRNA molecules is under separate promoter control or some combination thereof. The proteins of this invention can be expressed from a multi-gene expression system in which one or more proteins of the TIC4747 protein toxin class are expressed from a common nucleotide segment which also contains other open reading frames and promoters, depending on the type of expression system selected. In one example, a plant multi-gene expression system can utilize multiply-linked expression cassettes, each cassette expressing a different protein or other agent such as one or more dsRNA molecules. Yet in another example, a plant multi-gene expression system can utilize multiply-unlinked expression cassettes each expressing a different protein or other agent such as one or more dsRNA molecules.

Recombinant polynucleotides or recombinant DNA constructs comprising TIC4747 or a related family member protein encoding sequence can be delivered to host cells by vectors, e.g., a plasmid, baculovirus, synthetic chromosome, virion, cosmid, phagemid, phage, or viral vector. Such vectors can be used to achieve stable or transient expression of TIC4747 or a related family member protein encoding sequence in a host cell, or subsequent expression of the encoded polypeptide. An exogenous recombinant polynucleotide or recombinant DNA construct that comprises TIC4747 or a related family member protein encoding sequence and that is introduced into a host cell is referred herein as a “transgene.”

Transgenic bacteria, transgenic plant cells, transgenic plants, and transgenic plant parts that contain a recombinant polynucleotide that expresses any one or more of TIC4747 or a related family member protein encoding sequence are provided herein. The term “bacterial cell” or “bacterium” can include, but is not limited to, an Agrobacterium, Rhizobium, Bacillus, Brevibacillus, Escherichia, Pseudomonas, Klebsiella, and Erwinia wherein the Bacillus species can be a Bacillus cereus or a Bacillus thuringiensis, the Brevibacillus can be a Brevibacillus laterosperous, and the Escherichia can be an Escherichia coli cell. The term “plant cell” or “plant” can include but is not limited to a monocot, a dicot, an alfalfa, banana, barley, bean, broccoli, cabbage, brassica, carrot, cassava, castor, cauliflower, celery, chickpea, Chinese cabbage, citrus, coconut, coffee, corn, clover, cotton, a cucurbit, cucumber, Douglas fir, eggplant, eucalyptus, flax, garlic, grape, hops, leek, lettuce, Loblolly pine, millets, melons, nut, oat, olive, onion, ornamental, palm, pasture grass, pea, peanut, pepper, pigeon pea, pine, potato, poplar, pumpkin, Radiata pine, radish, rapeseed, rice, rootstocks, rye, safflower, shrub, sorghum, Southern pine, soybean, spinach, squash, strawberry, sugar beet, sugarcane, sunflower, sweet corn, sweet gum, sweet potato, switchgrass, tea, tobacco, tomato, triticale, turf grass, watermelon, and wheat plant cell or plant. In certain embodiments, transgenic plants and transgenic plant parts regenerated from a transgenic plant cell are provided. In certain embodiments, the transgenic plants can be obtained from a transgenic seed, by cutting, snapping, grinding or otherwise disassociating the part from the plant. In certain embodiments, the plant part can be a seed, a boll, a leaf, a flower, a stem, a root, or any portion thereof, or a non-regenerable portion of a transgenic plant part. As used in this context, a “non-regenerable” portion of a transgenic plant part is a portion that cannot be induced to form a whole plant or that cannot be induced to form a whole plant that is capable of sexual and/or asexual reproduction. In certain embodiments, a non-regenerable portion of a plant part is a portion of a transgenic seed, boll, leaf, flower, stem, or root.

Methods of making transgenic plants that comprise insect or Hemiptera or Lepidoptera-inhibitory amounts of TIC4747 or a related family member are provided. Such plants can be made by introducing a recombinant polynucleotide that encodes any of the TIC4747 or related family member proteins provided herein into a plant cell, and selecting a plant derived from the plant cell that expresses a Lepidoptera-inhibitory or Hemiptera-inhibitory amount of the protein. Plants can be derived from the plant cells by regeneration, seed, pollen, or meristem transformation techniques. Methods for transforming plants are known in the art.

Processed plant products, wherein the processed product comprises a detectable amount of a protein from the TIC4747 protein toxin class, an insect inhibitory fragment thereof, or any distinguishing portion thereof, are also disclosed herein. In certain embodiments, the processed product is selected from the group consisting of plant parts, plant biomass, oil, meal, sugar, animal feed, flour, flakes, bran, lint, hulls, processed seed and seed. In certain embodiments, the processed plant product is non-regenerable. The plant product can comprise commodity or other products of commerce derived from a transgenic plant or transgenic plant part, where the commodity or other products can be tracked through commerce by detecting nucleotide segments or expressed RNA or proteins that encode or comprise distinguishing portions of a protein from the TIC474 protein toxin class.

Plants expressing a protein from the TIC4747 protein toxin class can be crossed by breeding transgenic events expressing other toxin proteins or expressing other transgenic traits such as herbicide tolerance genes, genes conferring yield or stress tolerance traits, and the like, or such traits can be combined in a single vector so that the traits are all linked.

As described further in the Examples of this application, synthetic or artificial sequences encoding TIC4747 and related family member insecticidal proteins were designed for use in plants. Exemplary synthetic nucleotide sequences that were designed for use in plants are set forth in SEQ ID NO:11 (TIC4747PL), SEQ ID NO:13 (TIC7181PL), SEQ ID NO:15 (TIC4904PL), SEQ ID NO:17 (TIC6547PL), and SEQ ID NO:19 (TIC4006PL).

For expression in plant cells, the TIC4747 and related family member insecticidal proteins can be expressed to reside in the cytosol or targeted to various organelles of the plant cell. For example, targeting a protein to the chloroplast may result in increased levels of expressed protein in a transgenic plant while preventing off-phenotypes from occurring. Targeting may also result in an increase in pest resistance efficacy in the transgenic event. A target peptide or transit peptide is a short (3-70 amino acids long) peptide chain that directs the transport of a protein to a specific region in the cell, including the nucleus, mitochondria, endoplasmic reticulum (ER), chloroplast, apoplast, peroxisome and plasma membrane. Some target peptides are cleaved from the protein by signal peptidases after the proteins are transported. For targeting to the chloroplast, proteins contain transit peptides which are around 40-50 amino acids. For descriptions of the use of chloroplast transit peptides, see U.S. Pat. Nos. 5,188,642 and 5,728,925. Many chloroplast-localized proteins are expressed from nuclear genes as precursors and are targeted to the chloroplast by a chloroplast transit peptide (CTP). Examples of such isolated chloroplast proteins include, but are not limited to, those associated with the small subunit (SSU) of ribulose-1,5,-bisphosphate carboxylase, ferredoxin, ferredoxin oxidoreductase, the light-harvesting complex protein I and protein II, thioredoxin F, enolpyruvyl shikimate phosphate synthase (EPSPS), and transit peptides described in U.S. Pat. No. 7,193,133. It has been demonstrated in vivo and in vitro that non-chloroplast proteins may be targeted to the chloroplast by use of protein fusions with a heterologous CTP and that the CTP is sufficient to target a protein to the chloroplast. Incorporation of a suitable chloroplast transit peptide such as the Arabidopsis thaliana EPSPS CTP (CTP2) (see, Klee et al., Mol. Gen. Genet. 210:437-442, 1987) or the Petunia hybrida EPSPS CTP (CTP4) (see, della-Cioppa et al., Proc. Natl. Acad. Sci. USA 83:6873-6877, 1986) has been shown to target heterologous EPSPS protein sequences to chloroplasts in transgenic plants (see, U.S. Pat. Nos. 5,627,061; 5,633,435; and 5,312,910; and EP 0218571; EP 189707; EP 508909; and EP 924299). For targeting the TIC4747 and related family member insecticidal proteins to the chloroplast, a sequence encoding a chloroplast transit peptide is placed 5′ in operable linkage and in frame to a synthetic coding sequence encoding the TIC4747PL and related family member insecticidal protein that has been designed for optimal expression in plant cells.

Expression cassettes and vectors containing these synthetic or artificial nucleotide sequences can be constructed and introduced into corn, cotton, and soybean plant cells in accordance with transformation methods and techniques which are known in the art. Transformed cells are regenerated into transformed plants that are observed to be expressing the TIC4747PL and related family member insecticidal protein. To test pesticidal activity, bioassays are performed in the presence of Hemipteran pest nymphs using, for example developing soybean pods obtained from the transformed soybean plants or leaf discs from transformed corn plants.

TIC4747 and related family member protein-encoding sequences and sequences having a substantial percentage identity to TIC4747 and related family member protein-encoding sequences can be identified using methods known to those of ordinary skill in the art such as polymerase chain reaction (PCR), thermal amplification and hybridization. For example, the proteins of the TIC4747 and related family member protein toxin class can be used to produce antibodies that bind specifically to this class of proteins, and can be used to screen for and to find other members of the class.

Further, nucleotide sequences encoding the TIC4747 protein toxin class (and reverse complement sequences) can be used as probes and primers for screening to identify other members of the class using thermal-cycle or isothermal amplification and hybridization methods. Nucleotide sequence homologs, e.g., insecticidal proteins encoded by nucleotide sequences that hybridize to each or any of the sequences disclosed in this application under stringent hybridization conditions, are an embodiment of the present invention. The invention also provides a method for detecting a first nucleotide sequence that hybridizes to a second nucleotide sequence, wherein the first nucleotide sequence (or its reverse complement sequence) encodes an insecticidal protein or insecticidal fragment thereof and hybridizes under stringent hybridization conditions to the second nucleotide sequence. In such case, the second nucleotide sequence can be any of the nucleotide sequences disclosed in the TIC4747 protein toxin class under stringent hybridization conditions. Nucleotide coding sequences hybridize to one another under appropriate hybridization conditions, such as stringent hybridization conditions, and the proteins encoded by these nucleotide sequences cross react with antiserum raised against any one of the other proteins. Stringent hybridization conditions, as defined herein, comprise at least hybridization at 42° C. followed by two washes for five minutes each at room temperature with 2×SSC, 0.1% SDS, followed by two washes for thirty minutes each at 65° C. in 0.5×SSC, 0.1% SDS. Washes at even higher temperatures constitute even more stringent conditions, e.g., hybridization conditions of 68° C., followed by washing at 68° C., in 2×SSC containing 0.1% SDS.

One skilled in the art will recognize that, due to the redundancy of the genetic code, many other sequences are capable of encoding such related proteins, and those sequences, to the extent that they function to express insecticidal proteins either in Bt strains or in plant cells, are embodiments of the present invention, recognizing of course that many such redundant coding sequences will not hybridize under these conditions to the native Bt sequences encoding TIC4747 and related family members. This application contemplates the use of these, and other identification methods known to those of ordinary skill in the art, to identify TIC4747 and related family member protein-encoding sequences and sequences having a substantial percentage identity to TIC4747 and related family member protein-encoding sequences.

This disclosure also contemplates the use of molecular methods known in the art to engineer and clone commercially useful proteins comprising chimeras of proteins from pesticidal proteins; e.g., the chimeras may be assembled from segments of TIC4747 and related family member proteins to derive additional useful embodiments including assembly of segments of TIC4747 and related family member proteins with segments of diverse proteins different from TIC4747 and related proteins. Proteins of the TIC4747 protein toxin class may be subjected to alignment to each other and to other Bt pesticidal proteins (whether or not these are closely or distantly related phylogenetically), and segments of each such protein may be identified that are useful for substitution between the aligned proteins, resulting in the construction of chimeric proteins. Such chimeric proteins can be subjected to pest bioassay analysis and characterized for the presence or absence of increased bioactivity and/or expanded target pest spectrum compared to the parent proteins from which each such segment in the chimera was derived. The pesticidal activity of the polypeptides may be further engineered for activity to a particular pest or to a broader spectrum of pests by swapping domains or segments with other proteins or by using directed evolution methods known in the art.

Methods of controlling insects, in particular Hemipteran and Lepidopteran infestations of crop plants, with proteins from the TIC4747 toxin protein class are also disclosed in this application. Such methods can comprise growing a plant comprising an insect- or Hemiptera- or Lepidoptera-inhibitory amount of a protein from the TIC4747 toxin protein class. In certain embodiments, such methods can further comprise any one or more of: (i) applying any composition comprising or encoding a protein of the TIC4747 protein toxin class to the plant or a seed that gives rise to the plant; and (ii) transforming the plant or a plant cell that gives rise to the plant with a polynucleotide encoding a protein of the TIC4747 protein toxin class. In general, it is contemplated that any protein in the TIC4747 protein toxin class can be provided in a composition, provided in a microorganism, or provided in a transgenic plant to confer insect inhibitory activity against Lepidopteran insects.

In certain embodiments, a recombinant polypeptide of the TIC4747 protein toxin class is the insecticidally active ingredient of an insect inhibitory composition prepared by culturing recombinant Bt cells under conditions to express and produce proteins of the TIC4747 protein toxin class. Such a composition can be prepared by desiccation, lyophilization, homogenization, extraction, filtration, centrifugation, sedimentation, or concentration of a culture of recombinant Bt cells expressing/producing said recombinant polypeptide. Such a process can result in a Bt or other entomopathogenic bacterial cell extract, cell suspension, cell homogenate, cell lysate, cell supernatant, cell filtrate, or cell pellet. By obtaining the recombinant polypeptides so produced, a composition that includes the recombinant polypeptides can include bacterial cells, bacterial spores, and parasporal inclusion bodies and can be formulated for various uses, including as agricultural insect inhibitory spray products or as insect inhibitory formulations in diet bioassays.

The possibility for insects to develop resistance to certain insecticides has been documented in the art. One insect resistance management strategy is to employ transgenic crops that express two distinct insect inhibitory agents that operate through different modes of action. Therefore, any insects with resistance to either one of the insect inhibitory agents can be controlled by the other insect inhibitory agent. Another insect resistance management strategy employs the use of plants that are not protected to the target pest species to provide a refuge for such unprotected plants. One particular example is described in U.S. Pat. No. 6,551,962, which is incorporated by reference in its entirety.

In one embodiment, to reduce the likelihood of resistance development, an insect inhibitory composition or transgenic plant comprising one or more proteins from the TIC4747 protein toxin class can further comprise at least one additional polypeptide that exhibits insect inhibitory activity against the same or other susceptible target insect species, but is different from the TIC4747 protein toxin. Possible additional polypeptides for such a composition include an insect inhibitory protein, an insect inhibitory dsRNA molecule, and an ancillary protein. One example for the use of such ribonucleotide sequences to control insect pests is described in U.S. Patent Publication 2006/0021087. Such additional polypeptide for the control of Lepidopterans may be selected from the group consisting of a Lepidopteran insect inhibitory protein, such as, but not limited to, Cry1A (U.S. Pat. No. 5,880,275), Cry1Ab, Cry1Ac, Cry1A.105, Cry1Ae, Cry1B (U.S. patent Ser. No. 10/525,318), Cry1C (U.S. Pat. No. 6,033,874), Cry1D, Cry1 Da, Cry1E, Cry1F, and Cry1A/F chimeras (U.S. Pat. Nos. 7,070,982; 6,962,705; and 6,713,063), Cry1G, Cry1H, Cry1I, Cry1J, Cry1K, Cry1L, Cry2A, Cry2Ab (U.S. Pat. No. 7,064,249), Cry2Ae, Cry4B, Cry6, Cry7, Cry8, Cry9, Cry15, Cry43A, Cry43B, Cry51Aa1, ET66, TIC400, TIC800, TIC834, TIC1415, TIC2160 (International Application Publication WO2016/061392(A2)), Vip3A, VIP3Ab, VIP3B, AXMI-001, AXMI-002, AXMI-030, AXMI-035, AND AXMI-045 (U.S. Patent Publication 2013-0117884 A1), AXMI-52, AXMI-58, AXMI-88, AXMI-97, AXMI-102, AXMI-112, AXMI-117, AXMI-100 (U.S. Patent Publication 2013-0310543 A1), AXMI-115, AXMI-113, AXMI-005 (U.S. Patent Publication 2013-0104259 A1), AXMI-134 (U.S. Patent Publication 2013-0167264 A1), AXMI-150 (U.S. Patent Publication 2010-0160231 A1), AXMI-184 (U.S. Patent Publication 2010-0004176 A1), AXMI-196, AXMI-204, AXMI-207, axmi209 (U.S. Patent Publication 2011-0030096 A1), AXMI-218, AXMI-220 (U.S. Patent Publication 2014-0245491 A1), AXMI-221z, AXMI-222z, AXMI-223z, AXMI-224z, AXMI-225z (U.S. Patent Publication 2014-0196175 A1), AXMI-238 (U.S. Patent Publication 2014-0033363 A1), AXMI-270 (U.S. Patent Publication 2014-0223598 A1), AXMI-345 (U.S. Patent Publication 2014-0373195 A1), AXMI-335 (International Application Publication WO2013/134523(A2)), DIG-3 (U.S. Patent Publication 2013-0219570 A1), DIG-5 (U.S. Patent Publication 2010-0317569 A1), DIG-11 (U.S. Patent Publication 2010-0319093 A1), AfIP-1A and derivatives thereof (U.S. Patent Publication 2014-0033361 A1), AfIP-1B and derivatives thereof (U.S. Patent Publication 2014-0033361 A1), PIP-1APIP-1B (U.S. Patent Publication 2014-0007292 A1), PSEEN3174 (U.S. Patent Publication 2014-0007292 A1), AECFG-592740 (U.S. Patent Publication 2014-0007292 A1), DIG-657 (International Application Publication WO2015/195594(A2)), Pput_1063 (U.S. Patent Publication 2014-0007292 A1), Pput_1064 (U.S. Patent Publication 2014-0007292 A1), GS-135 and derivatives thereof (U.S. Patent Publication 2012-0233726 A1), GS153 and derivatives thereof (U.S. Patent Publication 2012-0192310 A1), GS154 and derivatives thereof (U.S. Patent Publication 2012-0192310 A1), GS155 and derivatives thereof (U.S. Patent Publication 2012-0192310 A1), SEQ ID NO: 2 and derivatives thereof as described in U.S. Patent Publication 2012-0167259 A1, SEQ ID NO: 2 and derivatives thereof as described in U.S. Patent Publication 2012-0047606 A1, SEQ ID NO: 2 and derivatives thereof as described in U.S. Patent Publication 2011-0154536 A1, SEQ ID NO: 2 and derivatives thereof as described in U.S. Patent Publication 2011-0112013 A1, SEQ ID NO: 2 and 4 and derivatives thereof as described in U.S. Patent Publication 2010-0192256 A1, SEQ ID NO: 2 and derivatives thereof as described in U.S. Patent Publication 2010-0077507 A1, SEQ ID NO: 2 and derivatives thereof as described in U.S. Patent Publication 2010-0077508 A1, SEQ ID NO: 2 and derivatives thereof as described in U.S. Patent Publication 2009-0313721 A1, SEQ ID NO: 2 or 4 and derivatives thereof as described in U.S. Patent Publication 2010-0269221 A1, SEQ ID NO: 2 and derivatives thereof as described in U.S. Pat. No. 7,772,465 (B2), CF161_0085 and derivatives thereof as described in WO2014/008054 A2, Lepidopteran toxic proteins and their derivatives as described in US Patent Publications US2008-0172762 A1, US2011-0055968 A1, and US2012-0117690 A1; SEQ ID NO: 2 and derivatives thereof as described in U.S. Pat. No. 7,510,878(B2), SEQ ID NO: 2 and derivatives thereof as described in U.S. Pat. No. 7,812,129(B1); DIG-911 and DIG-180 as described in US Patent Publication No. 2015-0264940A1, and the like.

Where the target insect pest is a Hemipteran insect pest, such additional polypeptide may be selected from the group consisting of Hemipteran-active proteins such as, but not limited to, TIC1415 (US Patent Publication 2013-0097735 A1), TIC807 (U.S. Pat. No. 8,609,936), TIC834 (U.S. Patent Publication 2013-0269060 A1), AXMI-036 (U.S. Patent Publication 2010-0137216 A1), and AXMI-171 (U.S. Patent Publication 2013-0055469 A1).

In other embodiments, such composition/formulation can further comprise at least one additional polypeptide that exhibits insect inhibitory activity to an insect that is not inhibited by an otherwise insect inhibitory protein of the present invention to expand the spectrum of insect inhibition obtained. For example, for the control of Coleopteran pests, combinations of insect inhibitory proteins of the present invention can be used with Coleopteran-active proteins such as, but not limited to, Cry3Bb (U.S. Pat. No. 6,501,009), Cry1C variants, Cry3A variants, Cry3, Cry3B, Cry34/35, 5307, AXMI134 (U.S. Patent Publication 2013-0167264 A1) AXMI-184 (U.S. Patent Publication 2010-0004176 A1), AXMI-205 (U.S. Patent Publication 2014-0298538 A1), AXMI-207 (U.S. Patent Publication 2013-0303440 A1), AXMI-218, AXMI-220 (U.S. Patent Publication 20140245491A1), AXMI-221z, AXMI-223z (U.S. Patent Publication 2014-0196175 A1), AXMI-279 (U.S. Patent Publication 2014-0223599 A1), AXMI-R1 and variants thereof (U.S. Patent Publication 2010-0197592 A1, TIC407, TIC417, TIC431, TIC807, TIC853, TIC901, TIC1201, TIC3131, DIG-10 (U.S. Patent Publication 2010-0319092 A1), eHIPs (U.S. Patent Application Publication No. 2010/0017914), IP3 and variants thereof (U.S. Patent Publication 2012-0210462 A1), PHI-4 variants (U.S. Patent Application Publication 2016-0281105 A1), PIP-72 variants (WO 2016-144688 A1), PIP-45 variants, PIP-64 variants, PIP-74 variants, PIP-75 variants, and PIP-77 variants (WO 2016-144686 A1), DIG-305 (WO 2016109214 A1), PIP-47 variants (U.S. Patent Publication 2016-0186204 A1), DIG-17, DIG-90, DIG-79 (WO 2016-057123 A1), DIG-303 (WO 2016-070079 A1), and ω̅-Hexatoxin-Hvla (U.S. Patent Application Publication US2014-0366227 A1).

Additional polypeptides for the control of Coleopteran, Lepidopteran, and Hemipteran insect pests can be found on the Bacillus thuringiensis toxin nomenclature website maintained by Neil Crickmore (on the World Wide Web at btnomenclature.info).

Other embodiments such as topically applied pesticidal chemistries that are designed for controlling pests that are also controlled by the proteins disclosed herein to be used with proteins in seed treatments, spray on, drip on, or wipe on formulations can be applied directly to the soil (a soil drench), applied to growing plants expressing the proteins disclosed herein, or formulated to be applied to seed containing one or more transgenes encoding one or more of the proteins disclosed. Such formulations for use in seed treatments can be applied with various stickers and tackifiers known in the art. Such formulations can contain pesticides that are complementary in mode of action with the proteins disclosed, so that the formulation pesticides act through a different mode of action to control the same or similar pests that can be controlled by the proteins disclosed, or that such pesticides act to control pests within a broader host range, such as Lepidopteran or Hemipteran species or other plant pest species such as Coleopteran species that are not effectively controlled.

The aforementioned composition/formulation can further comprise an agriculturally-acceptable carrier, such as a bait, a powder, dust, pellet, granule, spray, emulsion, a colloidal suspension, an aqueous solution, a Bacillus spore/crystal preparation, a seed treatment, a recombinant plant cell/plant tissue/seed/plant transformed to express one or more of the proteins, or bacterium transformed to express one or more of the proteins. Depending on the level of insect inhibitory or insecticidal inhibition inherent in the recombinant polypeptide and the level of formulation to be applied to a plant or diet assay, the composition/formulation can include various by weight amounts of the recombinant polypeptide, e.g. from 0.0001% to 0.001% to 0.01% to 1% to 99% by weight of the recombinant polypeptide.

### EXAMPLES

In view of the foregoing, those of skill in the art should appreciate that changes can be made in the specific aspects which are disclosed and still obtain a like or similar result without departing from the spirit and scope of the invention. Thus, specific details disclosed herein are not to be interpreted as limiting. It should be understood that the entire disclosure of each reference cited herein is incorporated by reference in its entirety within the disclosure of this application.

### Example 1

### Discovery of Novel Bt Genes

This Example describes the discovery of the pesticidal proteins TIC4747, TIC7181, TIC4904, TIC6547, and TIC4006.

A sequence encoding a novel Bacillus thuringiensis (Bt) pesticidal protein was identified, cloned, sequence confirmed, and tested in bioassay. The pesticidal protein TIC4747, presented herein as SEQ ID NOs 1 (Bt coding sequence) and 2 (protein), was isolated from Bacillus thuringiensis strain CFB007452. High throughput sequencing and bioinformatics were used to screen Bt genomes for genes (open reading frames) encoding proteins exhibiting similarity to TIC4747. Four related toxin proteins were identified in this screen and are presented in Table 3, along with the corresponding Bt strain and percent identity to TIC4747.

TABLE 3
TIC4747 and related toxin proteins.

| | | | | % Identity to |
|---|---|---|---|---|
| | | Coding | Protein | TIC4747 |
| Toxin | Strain | Sequence | Sequence | protein |
| TIC4747 | CFB007452 | 1 | 2 | — |
| TIC4904 | CFB007432 | 3 | 4 | 99.9 |
| TIC4006 | WC12466 | 5 | 6 | 94.2 |
| TIC6547 | CFB231019 | 7 | 8 | 93.1 |
| TIC7181 | EG9737 | 9 | 10 | 82.3 |

Nucleotide segments encoding TIC4747 and related family members were made by polymerase chain reaction (PCR) amplification using genomic DNA from the corresponding strains and cloned into Bt plasmid expression vectors which comprised a sporulation stage promoter operably linked to each toxin coding sequence.

### Example 2

### Bioassay of TIC4747 and Related Family Members Against Insect Pests

TIC4747 and related family members were expressed in Bt and E. coli, and assayed for toxicity to various species of Lepidoptera, Coleoptera, Hemiptera, and Diptera.

Bacillus thuringiensis host cells were transformed with plasmid expression vectors comprising the insect toxin coding sequences presented in Table 3 of Example 1. The transformed Bt cells were grown in liquid culture, harvested during the sporulation growth stage, and protein was extracted from the cell lysate. In addition, Histadine tagged proteins, TIC4747_His (SEQ ID NO:22, encoded by SEQ ID NO:21), TIC4904_His (SEQ ID NO:24, encoded by SEQ ID NO:23), TIC6547_His (SEQ ID NO:26, encoded by SEQ ID NO:25), and TIC4006_His (SEQ ID NO:28, encoded by SEQ ID NO:27) were prepared in E. coli cells using methods known in the art.

Preparations of TIC4747, TIC4006, TIC4904, and TIC6547 were presented in an insect diet bioassay against the Lepidopteran pest species Fall Armyworm (FAW, Spodoptera frugiperda), Corn Earworm (CEW, Helicoverpa zea), European Corn Borer (ECB, Ostrinia nubilalis), Southwestern Corn borer (SWC, Diatraea grandiosella), Soybean Looper (SL, Chrysodeixis includens), Velvetbean Caterpillar (VBC, Anticarsia gemmatalis), Tobacco Budworm (TBW, Heliothis virescens), Black Cutworm (BCW, Agrotis ipsilon), Southern Armyworm (SAW, Spodoptera eridania), and Diamondback Moth (DBM, Plutella xylostella); the Coleopteran pest species Western Corn Rootworm (WCR, Diabrotica virgifera LeConte) and Colorado Potato Beetle (CPB, Leptinotarsa decemlineata); the Hemipteran pest species Tarnished Plant Bug (TPB, Lygus lineolaris), Western Tarnished Plant Bug (WTPB, Lygus hesperus), Southern Green Stink Bug (SGB, Nezara viridula), Neotropical Brown Stink Bug (NBSB, Euschistus heros). Preparations of TIC4747, TIC4006, and TIC4904 were also presented in an insect diet bioassay against the Dipteran pest species Yellow Fever Mosquito (YFM, Aedes aegypti). Preparations of TIC7181 were presented in an insect diet bioassay against the pests, CEW, ECB, FAW, SAW, SBL, SWCB, TBW, VBC, CPB, TPB, and WTB.

The pesticidal toxin TIC4747 demonstrated activity against Tarnished Plant Bug, Western Tarnished Plant Bug, Green Stink Bug, and Neotropical Brown Stink Bug. The pesticidal toxin TIC6547 demonstrated activity against Diamondback Moth. The pesticidal toxin TIC7181 demonstrated activity against Tarnished Plant Bug.

### Example 3

### Design of Synthetic Coding Sequences Encoding TIC4747PL and Related Family Members for Expression in Plant Cells

Synthetic coding sequences are designed for use in expression of the encoded protein in plants, and are cloned into binary plant transformation vectors, and used to transform plant cells. The synthetic sequences are synthesized, according to methods generally described in U.S. Pat. No. 5,500,365 to avoid certain inimical problem sequences such as ATTTA and A/T rich plant polyadenylation sequences, while preserving the amino acid sequence of the original protein. The synthetic coding sequences presented in Table 4 below encode plant version proteins of TIC4747 and related family members wherein an additional alanine amino acid residue is incorporated immediately following the initiating methionine amino acid residue in the native toxin protein sequence to improve plant expression of the toxin.

TABLE 4
Synthetic coding sequences used for expression of TIC4747PL
and related family members in plant cells.

| | Plant Coding | |
|---|---|---|
| | Sequence | Plant Protein |
| Toxin | SEQ ID NO: | SEQ ID NO: |
| TIC4747PL | 11 | 12 |
| TIC7181PL | 13 | 14 |
| TIC4904PL | 15 | 16 |
| TIC6547PL | 17 | 18 |
| TIC4006PL | 19 | 20 |

The synthetic coding sequences encoding TIC4747PL and related family members are cloned into binary plant transformation vectors comprising a first transgene cassette for expression of the TIC4747PL toxin or related family members and a second transgene cassette for selection of transformed plant cells. This first cassette comprises a plant expressible promoter operably linked 5′ to an optional intron, which is operably linked 5′ to a plastid targeted or untargeted TIC4747PL toxin or related family member coding sequence, which is operably linked 5′ to a 3′ UTR. The second transgene cassette for selection of transformed plant cells uses either glyphosate or an antibiotic such as spectinomycin.

### Example 4

### Assay of TIC4747PL and Related Family Members Against Hemipteran Pests in Stably Transformed Soybean Plants

This Example describes the assay of activity against Hemipteran insect pests in soybean plants stably transformed to express TIC4747PL or related family member toxins.

Soybean plants are transformed using binary plant transformation vectors as described in Example 3. The transformed soybean plant cells are induced to form whole plants. Assay for activity against the Hemipteran pests is performed using a variety of techniques which will depend upon the species of Hemipteran pests and the preferred target tissue of that pest. For example, the Hemipteran pest species of Stink Bugs typically feed on the developing seeds and pods of the soybean plant. To assay for activity against Stink Bugs, R5 stage pods are harvested from the transgenic soybean plants expressing TIC4747PL or related family members and placed in a covered Petri dish or large multi-well plate containing a layer of either agar or wet paper to provide humidity to the feeding environment. Second instar Stink Bug nymphs are placed in the Petri dish or large multi-well plate. A cover providing for the exchange of oxygen while preventing desiccation is placed over the feeding environment. The Stink Bug nymphs are allowed to feed for several days. Measurements of stunting and mortality are taken and compared to Stink Bugs nymphs feeding on pods from untransformed soybean plants.

Alternatively, assay of activity can also be performed on whole stably transformed plants. Transformed plants expressing a protein from the TIC4747 protein toxin class are grown in a growth chamber or in the greenhouse. At R5 stage, the plants are enclosed in a cage made from breathable plastic “pollination” sheets (Vilutis and Company Inc, Frankfort, IL). The sheet sleeves are secured to the main stem just above the soil surface using a Velcro® tie. Each plant is infested with a specific number of second instar Stink Bug nymphs. The nymphs are released into each individual cage through a small slit on the cage side and then the cage is securely closed ensuring the insects won't escape; and allowed to feed on the soybean pods for several days to a week or more. Observations are taken each day to determine measurements of stunting and mortality. At the end of the feeding period, the live and dead nymphs are collected. The plants are cut below the cages and moved to a laboratory where the insects are collected for each plant. Before opening the cage, the plants are vigorously shaken to ensure all of the insects fall off from their feeding sites to the base of the cage. Then the cage base is opened and all plant material is removed and placed on a black sheet. The insects can be collected using an aspirator or some other means. The number of insects and their developmental stage is recorded for each plant. The number and developmental stage of dead nymphs is also recorded. These measurements are compared to the measurements obtained from the negative control, un-transformed plants.

Delays in development of the Stink Bug nymphs (stunting) or mortality are interpreted as an indication of toxicity if, when compared to the un-transformed controls, there is a significant difference.

### Example 5

### Assay of TIC4747PL or Related Family Member Activity Against Hemipteran Pests in Stably Transformed Corn Plants

This Example describes the assay of activity against Hemipteran insect pests in corn plants stably transformed to express TIC4747PL or related family member toxins.

Corn plants are transformed using binary plant transformation vectors as described in Example 3 above. The transformed corn plant cells are induced to form whole plants. Assay for activity against the Hemipteran pests is performed using a variety of techniques which will depend upon the species of Hemipteran pest and the preferred target tissue of that pest. For example, the Hemipteran pest species of Stink Bugs typically feed on the young corn plants in late spring or early summer, resulting in holes in the leaf, and if severe, deformed plants. In late summer, Stink Bugs typically feed on the ear itself, directly destroying the kernels.

One method to assay for Stink Bug activity is to expose the Stink Bug nymphs to leaf discs derived from stably transformed corn plants expressing TIC4747PL or related family members in large multi-well plates. Second stage instar Stink Bug nymphs are placed in large multi-well plates with leaf discs derived from the stably transformed corn plants and allowed to feed for several days. Measurements of stunting and mortality are taken and compared to Stink Bug nymphs who have fed on un-transformed corn leaf discs.

Alternatively, whole transformed plants can be used to assay for Stink Bug activity. Stably transformed corn plants expressing TIC4747PL or related family members are enclosed in cages in a similar manner as described for soybean plants in Example 4 above. Second instar nymphs are introduced to V3 stage corn plants and allowed to feed for several days to a week. After the prescribed feeding period, the nymphs are collected as described in Example 4 above. Measurements of stunting and mortality are compared to un-transformed control plants.

To assay Stink Bug activity using stably transformed corn ears, a similar approach can be taken as that of assaying in V3 stage plants. The developing corn ears of stably transformed corn plants expressing TIC4747PL or related family members are encapsulated using sheets of material that permit the free exchange of air while preventing escape of the Stink Bug nymphs. The encapsulated ears are infested with second instar stage Stink Bug nymphs and allowed to feed on the developing kernels of the ear for several days to a week. Measurements of stunting and mortality are compared to un-transformed control plant ears.

All of the compositions disclosed and claimed herein can be made and executed without undue experimentation in light of the present disclosure. While the compositions of this invention have been described in terms of the foregoing illustrative embodiments, it will be apparent to those of skill in the art that variations, changes, modifications, and alterations may be applied to the composition described herein, without departing from the true concept, spirit, and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope, and concept of the invention as defined by the appended claims.

All publications and published patent documents cited in the specification are incorporated herein by reference to the same extent as if each individual publication or patent application was specifically and individually indicated to be incorporated by reference.

What is claimed is:

1. A recombinant nucleic acid molecule comprising a heterologous promoter operably linked to a polynucleotide segment encoding a pesticidal protein or fragment thereof, wherein:
a) said pesticidal protein comprises the amino acid sequence of SEQ ID NO:12, SEQ ID NO:2, SEQ ID NO:4, SEQ ID NO:6, SEQ ID NO:8, SEQ ID NO:10, SEQ ID NO:14, SEQ ID NO:16, SEQ ID NO:18, SEQ ID NO:20, SEQ ID NO:22, SEQ ID NO:24, SEQ ID NO:26, or SEQ ID NO:28; or
b) said pesticidal protein comprises an amino acid sequence having at least 62% amino acid sequence identity to the amino acid sequence of SEQ ID NO:12, SEQ ID NO:2, SEQ ID NO:4, SEQ ID NO:6, SEQ ID NO:8, SEQ ID NO:10, SEQ ID NO:14, SEQ ID NO:16, SEQ ID NO:18, SEQ ID NO:20, SEQ ID NO:22, SEQ ID NO:24, SEQ ID NO:26, or SEQ ID NO:28; or
c) said polynucleotide segment hybridizes under stringent hybridization conditions to a polynucleotide comprising the nucleotide sequence of SEQ ID NO:11, SEQ ID NO:1, SEQ ID NO:3, SEQ ID NO:5, SEQ ID NO:7, SEQ ID NO:9, SEQ ID NO:13, SEQ ID NO:15, SEQ ID NO:17, SEQ ID NO:19, SEQ ID NO:21, SEQ ID NO:23, SEQ ID NO:25, or SEQ ID NO:27 under hybridization conditions of 68° C., followed by washing at 68° C., in 2×SSC containing 0.1% SDS.

2. The recombinant nucleic acid molecule of claim 1:
a) comprising a sequence that functions to express the pesticidal protein in a plant or a pesticidal fragment thereof in a plant; or
b) wherein said molecule is expressed in a plant cell to produce a pesticidally effective amount of the pesticidal protein or a pesticidal fragment thereof; or
c) wherein said recombinant nucleic acid molecule is in operable linkage to a vector, and said vector is selected from the group consisting of a plasmid, phagemid, bacmid, cosmid, and a bacterial or yeast artificial chromosome.

3. The recombinant nucleic acid molecule of claim 1 present within a host cell, wherein said host cell is selected from the group consisting of a bacterial and a plant cell.

4. The recombinant nucleic acid molecule of claim 3, wherein the bacterial host cell is from a genus of bacteria selected from the group consisting of: Agrobacterium, Rhizobium, Bacillus, Brevibacillus, Escherichia, Pseudomonas, Klebsiella, and Erwinia; and wherein said Bacillus species is a Bacillus cereus or a Bacillus thuringiensis, said Brevibacillus is a Brevibacillus laterosperous, and said Escherichia is an Escherichia coli.

5. The recombinant nucleic acid of claim 3, wherein said plant cell is a dicotyledonous or a monocotyledonous plant cell.

6. The recombinant nucleic acid of claim 5, wherein said plant host cell is selected from the group consisting of an alfalfa, banana, barley, bean, broccoli, cabbage, brassica, carrot, cassava, castor, cauliflower, celery, chickpea, Chinese cabbage, citrus, coconut, coffee, corn, clover, cotton, a cucurbit, cucumber, Douglas fir, eggplant, eucalyptus, flax, garlic, grape, hops, leek, lettuce, Loblolly pine, millets, melons, nut, oat, olive, onion, ornamental, palm, pasture grass, pea, peanut, pepper, pigeonpea, pine, potato, poplar, pumpkin, Radiata pine, radish, rapeseed, rice, rootstocks, rye, safflower, shrub, sorghum, Southern pine, soybean, spinach, squash, strawberry, sugar beet, sugarcane, sunflower, sweet corn, sweet gum, sweet potato, switchgrass, tea, tobacco, tomato, triticale, turf grass, watermelon, and wheat plant cell.

7. The recombinant nucleic acid molecule of claim 1, wherein said protein exhibits activity against an insect species of the order of Hemiptera.

8. The recombinant nucleic acid molecule of claim 7, wherein said insect species is selected from the group consisting of: Chinavia hilaris, Chinavia marginata, Chinavia pensylvanica, Chlorochroa granulose, Chlorochroa kanei, Chlorochroa ligata, Chlorochroa lineate, Chlorochroa opuntiae, Chlorochroa persimilis, Chlorochroa rossiana, Chlorochroa sayi, Chlorochroa uhleri, Chlorochroa belfragii, Chlorochroa faceta, Chlorochroa osborni, Chlorochroa saucia, Chlorochroa senilis, Nezara viridula, Edessa meditabunda, Edessa bifida, Edessa florida, Euschistus heros, Euschistus acuminatus, Euschistus biformis, Euschistus conspersus, Euschistus crenator, Euschistus egglestoni, Euschistus ictericus, Euschistus inflatus, Euschistus latimarginatus, Euschistus obscures, Euschistus politus, Euschistus quadrator, Euschistus sevus, Euschistus strenuous, Euschistus tristigmus, Euschistus variolarius Halyomorpha halys, Thyanta accerra, Thyanta calceata, Thyanta custator, Thyanta pallidovirens, Thyanta perditor, Thyanta maculate, Thyanta pseudocasta Dichelops melacanthus, Dichelops avilapiresi, Dichelops bicolor, Dichelops dimidatus, Dichelops furcatus, Dichelops furcifrons, Dichelops lobatus, Dichelops miriamae, Dichelops nigrum, Dichelops peruanus, Dichelops phoenix, Dichelops saltensis, Piezodorus guildinni, Piezodorus lituratus Megacopta cribraria, Lygus hesperus, and Lygus lineolaris.

9. A plant comprising a recombinant nucleic acid molecule comprising a heterologous promoter operably linked to a polynucleotide segment encoding a pesticidal protein or fragment thereof, wherein:
a) said pesticidal protein comprises the amino acid sequence of SEQ ID NO:12, SEQ ID NO:2, SEQ ID NO:4, SEQ ID NO:6, SEQ ID NO:8, SEQ ID NO:10, SEQ ID NO:14, SEQ ID NO:16, SEQ ID NO:18, SEQ ID NO:20, SEQ ID NO:22, SEQ ID NO:24, SEQ ID NO:26, or SEQ ID NO:28; or
b) said pesticidal protein comprises an amino acid sequence having at least 62% amino acid sequence identity to the amino acid sequence of SEQ ID NO:12, SEQ ID NO:2, SEQ ID NO:4, SEQ ID NO:6, SEQ ID NO:8, SEQ ID NO:10, SEQ ID NO:14, SEQ ID NO:16, SEQ ID NO:18, SEQ ID NO:20, SEQ ID NO:22, SEQ ID NO:24, SEQ ID NO:26, or SEQ ID NO:28; or
c) said polynucleotide segment hybridizes under stringent hybridization conditions to a polynucleotide comprising the nucleotide sequence of SEQ ID NO:11, SEQ ID NO:1, SEQ ID NO:3, SEQ ID NO:5, SEQ ID NO:7, SEQ ID NO:9, SEQ ID NO:13, SEQ ID NO:15, SEQ ID NO:17, SEQ ID NO:19, SEQ ID NO:21, SEQ ID NO:23, SEQ ID NO:25, or SEQ ID NO:27 under hybridization conditions of 68° C., followed by washing at 68° C., in 2×SSC containing 0.1% SDS; or
d) said plant exhibits a detectable amount of said pesticidal protein, wherein the pesticidal protein comprises an amino acid sequence of SEQ ID NO:12, SEQ ID NO:2, SEQ ID NO:4, SEQ ID NO:6, SEQ ID NO:8, SEQ ID NO:10, SEQ ID NO:14, SEQ ID NO:16, SEQ ID NO:18, SEQ ID NO:20, SEQ ID NO:22, SEQ ID NO:24, SEQ ID NO:26, or SEQ ID NO:28.

10. The plant of claim 9, wherein said plant is a monocot plant or a dicot plant.

11. The plant of claim 10, wherein the plant is selected from the group consisting of an alfalfa, banana, barley, bean, broccoli, cabbage, brassica, carrot, cassava, castor, cauliflower, celery, chickpea, Chinese cabbage, citrus, coconut, coffee, corn, clover, cotton, a cucurbit, cucumber, Douglas fir, eggplant, eucalyptus, flax, garlic, grape, hops, leek, lettuce, Loblolly pine, millets, melons, nut, oat, olive, onion, ornamental, palm, pasture grass, pea, peanut, pepper, pigeon pea, pine, potato, poplar, pumpkin, Radiata pine, radish, rapeseed, rice, rootstocks, rye, safflower, shrub, sorghum, Southern pine, soybean, spinach, squash, strawberry, sugar beet, sugarcane, sunflower, sweet corn, sweet gum, sweet potato, switchgrass, tea, tobacco, tomato, triticale, turf grass, watermelon, and wheat.

12. A seed from the plant of claim 9, wherein said seed comprises said recombinant nucleic acid molecule.

13. An insect inhibitory composition comprising the recombinant nucleic acid molecule of claim 1.

14. The insect inhibitory composition of claim 13, further comprising a nucleotide sequence encoding at least one other pesticidal agent that is different from said pesticidal protein.

15. The insect inhibitory composition of claim 14, wherein said at least one other pesticidal agent is selected from the group consisting of an insect inhibitory protein, an insect inhibitory dsRNA molecule, and an ancillary protein.

16. The insect inhibitory composition of claim 15, wherein said at least one other pesticidal agent exhibits activity against one or more pest species of the orders Lepidoptera, Coleoptera, Hemiptera, Homoptera, or Thysanoptera.

17. The insect inhibitory composition of claim 16, wherein said at least one other pesticidal protein is selected from the group consisting of a Cry1A, Cry1Ab, Cry1Ac, Cry1A.105, Cry1Ae, Cry1B, Cry1C, Cry1C variants, Cry1D, Cry1E, Cry1F, Cry1A/F chimeras, Cry1G, Cry1H, Cry1I, Cry1J, Cry1K, Cry1L, Cry2A, Cry2Ab, Cry2Ae, Cry3, Cry3A variants, Cry3B, Cry4B, Cry6, Cry7, Cry8, Cry9, Cry15, Cry34, Cry35, Cry43A, Cry43B, Cry51Aa1, ET29, ET33, ET34, ET35, ET66, ET70, TIC400, TIC407, TIC417, TIC431, TIC800, TIC807, TIC834, TIC853, TIC900, TIC901, TIC1201, TIC1415, TIC3131, TIC2160, VIP3A, VIP3B, VIP3Ab, AXMI-001, AXMI-002, AXMI-030, AXMI-035, AXMI-036, AXMI-045, Axmi52, Axmi58, Axmi88, Axmi97, Axmi102, Axmi112, Axmi117, Axmi100, AXMI-115, AXMI-113, and AXMI-005, AXMI134, AXMI-150, Axmi171, AXMI-184, axmi196, axmi204, axmi207, axmi209, Axmi205, AXMI218, AXMI220, AXMI221z, AXMI222z, AXMI223z, AXMI224z and AXMI225z, AXMI238, AXMI270, AXMI279, AXMI335, AXMI345, AXMI-R1, and variants thereof, IP3 and variants thereof, DIG-3, DIG-5, DIG-10, DIG-11, DIG-657 protein, PHI-4 variants, PIP-72 variants, PIP-45 variants, PIP-64 varients, PIP-74 variants, PIP-77 variants, DIG-305, PIP-47 varients, DIG-17, DIG-90, DIG-79, and DIG-303.

18. A commodity product produced from the host cell of claim 3, said commodity product comprising a detectable amount of said recombinant nucleic acid molecule or pesticidal protein.

19. The commodity product of claim 18, selected from the group consisting of commodity corn bagged by a grain handler, corn flakes, corn cakes, corn flour, corn meal, corn syrup, corn oil, corn silage, corn starch, corn cereal, and the like, and corresponding soybean, rice, wheat, sorghum, pigeon pea, peanut, fruit, melon, and vegetable commodity products including, where applicable, juices, concentrates, jams, jellies, marmalades, and other edible forms of such commodity products containing a detectable amount of such polynucleotides and or polypeptides of this application, whole or processed cotton seed, cotton oil, lint, seeds and plant parts processed for feed or food, fiber, paper, biomasses, and fuel products such as fuel derived from cotton oil or pellets derived from cotton gin waste, whole or processed soybean seed, soybean oil, soybean protein, soybean meal, soybean flour, soybean flakes, soybean bran, soybean milk, soybean cheese, soybean wine, animal feed comprising soybean, paper comprising soybean, cream comprising soybean, soybean biomass, and fuel products produced using soybean plants and soybean plant parts.

20. A method of producing seed comprising the recombinant nucleic acid molecule of claim 1, said method comprising:
a) planting at least one seed comprising said recombinant nucleic acid molecule;
b) growing plants from said seed; and
c) harvesting seed from said plants, wherein said harvested seed comprises said recombinant nucleic acid molecule.

21. A plant resistant to insect infestation, wherein said plant comprises the recombinant nucleic acid molecule of claim 1.

22. A method for controlling a Lepidopteran species pest, and controlling a Hemipteran species infestation of a plant, said method comprising contacting the pest with an insecticidally effective amount of one or more pesticidal proteins comprising an amino acid sequence having at least 62% amino acid sequence identity to SEQ ID NO:12, SEQ ID NO:2, SEQ ID NO:4, SEQ ID NO:6, SEQ ID NO:8, SEQ ID NO:10, SEQ ID NO:14, SEQ ID NO:16, SEQ ID NO:18, SEQ ID NO:20, SEQ ID NO:22, SEQ ID NO:24, SEQ ID NO:26, or SEQ ID NO:28.


SEQUENCE LISTING (28 sequences)

SEQ ID NO: 1 - DNA, 3654 bp, Bacillus thuringiensis
  misc_feature 1..3654: note = The native nucleotide sequence encoding TIC4747 toxin protein obtained from Bacillus thuringiensis species CFB007452.
  source 1..3654: mol_type = unassigned DNA
atggatcaaaagattataaaaatgcgagaagcagtcaatgccttgttttccaataatcatttaaaattgaatattactgattacaatatagatcagactgcataccttgttgatagtatgtctgatgacgcatatcgacaagaaaaaatgatgtttctcgatcaaatcaaatttgcaaagcgcttgagccaaaaacgcaacctgttgaatcatggagattttgaaggatccaattggacaggtaagaatggatggaaaagaaataattatgtagttgtcgcatcggatcatcctatatttaaaggccgatatttacacataccaggtgcaacaaccgcgatgagtggcgcaatcattccgacttatgtatatcaaagtatagatgaatcgaagttaaaaccgtatacacgttatttggtacgagggtttgttggaaagagtcaagatttagcgttacttgtttcccggtataccaaagaagtgtacaagaaaatcaatgtaccaaatgataaagactacgatatgacatcgcatataaatagggaagagaatctatggcacaatagatatataaaagacacttcggttcaaaattcaatctctatgtgcaaaaatccacatgaatttacgtgtcatattgatataggggaactggatagaaagaaaggtcctggtataaccatcggttttcaaattggaacaacagatgggatggcaacattagataatatagaagtgatagaagcacatccgttaaccggatacgccttagcacgtatcgaaaaacgtgaacgtaaatggaaacaaaaatggctagagcatcgaatacaaatcgaaaaggctgtgcaaacagcgcaagaggtgattcgaaatttatttacatgcccacaacaaaatcaattgaactggatgacaacccgaaacgacattgcacatgcagaaacattgataaaagagatttcatatcggtatagccaactttcttgtggagatttccccatactaccagaagaggcgtatgacatccttcaacaactttcaactgcagttgaaaccgcaaaagcgttgtatacacaacgaaacgtggtgaataatggggattttcaagctggattatcgaattggcataggacagatggtgcagagatacaacaaattcagaatgcatcctctgttctaataattacagactgggctgcgaatatttcacaagacatgcgtgtagttgaaaaaggtagctatctgttgcgcgtaacagcgaaaaaagaagatgccggagaaggttatattacaattagtgattgtgccgcattgatagaaacattgacatttacaacgggggaatctgtggaaagtctgacacattctgatattcattcaaggctccataaacgctataataaaaaacacataaaaaaccatccttcagaagaatatgaaatagaatcggatcttcatttatttaatagggcggaacaaaacggttctctcccctctagctatgtaaccaaaacgatggaaatctttccggaaaccaatcgagtacgcattgaaattggagaaacaggtggaacatttatagtggaaagtgtggaattaattcgaatggaacagatgaacgaaacaaacaatccagatgtagatgttcaaattgtaatgaatgatacacccgctacacaatttgatccagtttcttttacagaatccacggtgaggcccagaaatgctcagtatgcatattctcatgattcaaatataggttatgaaaatcctaactggatggctgatatttcaggtgatactttatttactgatttatctatccctggtacacataatacaatggctctttatggaggagatattacacaatgtcaaacgatgtcactgaatacgcaattacatgtaggaattcgttatttagatattcgctgtaggcacatagaaaatgcttttgcgattcatcatggacctgtgtaccaaaatgcgatgtttggagatgtttgtattgccgtaaggaattttttgagaagcaaccctagtgaaacagtatttatgcggataaaagaagaacatacagcagaaaacaatacaagatctttttcagatacatttgcagattataagtctcaatatagcgacttattttgggattggacaggtgataacccaagattaagtgaaataagaggaaaagttgttgttttacaaaatttttcaggtggtaaatttggtatcaattacaatacattgaatactcaagatcaatatcatttaaatacaaactgggatttatatgataaatggctattcgtcaaagaacatttgtatgccgctgacaactcttataaaagtggccgtaaacaagtatatctgaattacctaagtggatcaggtggttcatttccttattttgttgcaagtggacatagtagtccaggtacagatgctccacaattatctacaggtctaacaacaccagcatttgcaagctggtatccggattttccacggggaagttgttttataggaatttgcacaatttactttgaaggaacaaatattcttacaagtcagtggatagagaaaaatgattttaaatatataggaatcatagctgctgattttccaggaagaacattaatttccaatattattagtctgaataaacttcttagcttagaaattaaaaatggtggtacctatcaaattgtttccgctttaaataatagtagtgttatagatatgagtctgagtggagatcgaaatgttcacctatggtccaataacggtactcttaatcaagtatggaaattcgtgtatgattcaaatagattggcataccaaattaaaagtctatccgatgaaaatttagtactaacttgggcttattatagtagtaatcgagataatgtaattgttgcttctaatcaaaatagcgatgagcaatattggatacctgagcgcacaggcgcatatcattattttaaaaatctcatcaatccctcgggagcattagatgtaagcggatcaggaacaacaaacggaacgaatattttgtattggagttataacagagcaacgaatcaaaaattcaaactggaagaagtaaatatacctggaggtcaagctgaaggtgtacttttatatgcagatgctaattatgtagggaaatctgtactactaacaaatagtgtctcaaaccttagagacgttggtatgaatgatatagccagttctataaaatttattggtccttatcaagctactctatatgaacatgataattttacaggtgcggcttttactctcacatctaatgttgcaaatttaaaagatgttggcatgaatgatacagttagttctataaaaattacaaagacatctggaggccgagctacaggtatatatttatatgcagatgctaattatgtaggcagatctgtatggttaacatctaatgttgcaaatttaaaagatattggcatgaatgatacagtcagttctgtagaaattgttggcgcatatcaagccactttatatggggatgccaattatacagggaaggcttataatctcactcataatgttacaaatttaaaagatgttggcatgaatgatatagtcagttccataaaaatttttagtgtgtaa

SEQ ID NO: 2 - protein, 1217 aa, Bacillus thuringiensis
  REGION 1..1217: note = MISC_FEATURE - The amino acid sequence of TIC4747.
MDQKIIKMREAVNALFSNNHLKLNITDYNIDQTAYLVDSMSDDAYRQEKMMFLDQIKFAKRLSQKRNLLNHGDFEGSNWTGKNGWKRNNYVVVASDHPIFKGRYLHIPGATTAMSGAIIPTYVYQSIDESKLKPYTRYLVRGFVGKSQDLALLVSRYTKEVYKKINVPNDKDYDMTSHINREENLWHNRYIKDTSVQNSISMCKNPHEFTCHIDIGELDRKKGPGITIGFQIGTTDGMATLDNIEVIEAHPLTGYALARIEKRERKWKQKWLEHRIQIEKAVQTAQEVIRNLFTCPQQNQLNWMTTRNDIAHAETLIKEISYRYSQLSCGDFPILPEEAYDILQQLSTAVETAKALYTQRNVVNNGDFQAGLSNWHRTDGAEIQQIQNASSVLIITDWAANISQDMRVVEKGSYLLRVTAKKEDAGEGYITISDCAALIETLTFTTGESVESLTHSDIHSRLHKRYNKKHIKNHPSEEYEIESDLHLFNRAEQNGSLPSSYVTKTMEIFPETNRVRIEIGETGGTFIVESVELIRMEQMNETNNPDVDVQIVMNDTPATQFDPVSFTESTVRPRNAQYAYSHDSNIGYENPNWMADISGDTLFTDLSIPGTHNTMALYGGDITQCQTMSLNTQLHVGIRYLDIRCRHIENAFAIHHGPVYQNAMFGDVCIAVRNFLRSNPSETVFMRIKEEHTAENNTRSFSDTFADYKSQYSDLFWDWTGDNPRLSEIRGKVVVLQNFSGGKFGINYNTLNTQDQYHLNTNWDLYDKWLFVKEHLYAADNSYKSGRKQVYLNYLSGSGGSFPYFVASGHSSPGTDAPQLSTGLTTPAFASWYPDFPRGSCFIGICTIYFEGTNILTSQWIEKNDFKYIGIIAADFPGRTLISNIISLNKLLSLEIKNGGTYQIVSALNNSSVIDMSLSGDRNVHLWSNNGTLNQVWKFVYDSNRLAYQIKSLSDENLVLTWAYYSSNRDNVIVASNQNSDEQYWIPERTGAYHYFKNLINPSGALDVSGSGTTNGTNILYWSYNRATNQKFKLEEVNIPGGQAEGVLLYADANYVGKSVLLTNSVSNLRDVGMNDIASSIKFIGPYQATLYEHDNFTGAAFTLTSNVANLKDVGMNDTVSSIKITKTSGGRATGIYLYADANYVGRSVWLTSNVANLKDIGMNDTVSSVEIVGAYQATLYGDANYTGKAYNLTHNVTNLKDVGMNDIVSSIKIFSV

SEQ ID NO: 3 - DNA, 3654 bp, Bacillus thuringiensis
  misc_feature 1..3654: note = The native nucleotide sequence encoding TIC7181 toxin protein obtained from Bacillus thuringiensis species EG9737.
  source 1..3654: mol_type = unassigned DNA
atggatcaaaagattataaaaatgcgagaagcagtcaatgccttgttttccaataatcatttaaaattgaatattactgattacaatatagatcagactgcataccttgttgatagtatgtctgatgacgcatatcgacaagaaaaaatgatgtttctcgatcaaatcaaatttgcaaagcgcttgagccaaaaacgcaacctgttgaatcatggagattttgaaggatccaattggacaggtaagaatggatggaaaagaaataattatgtagttgtcgcatcggatcatcctatatttaaaggccgatatttacacataccaggtgcaacaaccgcgatgagtggcgcaatcattccgacttatgtatatcaaagtatagatgaatcgaagttaaaaccgtatacacgttatttggtacgagggtttgttggaaagagtcaagatttagcgttacttgtttcccggtataccaaagaagtgtacaagaaaatcaatgtaccaaatgataaagactacgatatgacatcgcatataaatagggaagagaatctatggcacaatagatatataaaagacacttcggttcaaaattcaatctctatgtgcaaaaatccacatgaatttacgtgtcatattgatataggggaactggatagaaagaaaggtcctggtataaccatcggttttcaaattggaacaacagatgggatggcaacattagataatatagaagtgatagaagcacatccgttaaccggatacgccttagcacgtatcgaaaaacgtgaacgtaaatggaaacaaaaatggctagagcatcgaatacaaatcgaaaaggctgtgcaaacagcgcaagaggtgattcgaaatttatttacatgcccacaacaaaatcaattgaactggatgacaacccgaaacgacattgcacatgcagaaacattgataaaagagatttcatatcggtatagccaactttcttgtggagatttccccatactaccagaagaggcgtatgacatccttcaacaactttcaactgcagttgaaaccgcaaaagcgttgtatacacaacgaaacgtggtgaataatggggattttcaagctggattatcgaattggcataggacagatggtgcagagatacaacaaattcagaatgcatcctctgttctaataattacagactgggctgcgaatatttcacaagacatgcgtgtagttgaaaaaggtagctatctgttgcgcgtaacagcgaaaaaagaagatgccggagaaggttatattacaattagtgattgtgccgcattgatagaaacattgacatttacaacgggggaatctgtggaaagtctgacacattctgatattcattcaaggctccataaacgctataataaaaaacacataaaaaaccatccttcagaagaatatgaaatagaatcggatcttcatttatttaatagggcggaacaaaacggttctctcccctctagctatgtaaccaaaacgatggaaatctttccggaaaccaatcgaatacgcattgaaattggagaaacaggtggaacatttatagtggaaagtgtggaattaattcgaatggaacagatgaacgaaacaaacaatccagatgtagatgttcaaattgtaatgaatgatacacccgctacacaatttgatccagtttcttttacagaatccacggtgaggcccagaaatgctcagtatgcatattctcatgattcaaatataggttatgaaaatcctaactggatggctgatatttcaggtgatactttatttactgatttatctatccctggtacacataatacaatggctctttatggaggagatattacacaatgtcaaacgatgtcactgaatacgcaattacatgtaggaattcgttatttagatattcgctgtaggcacatagaaaatgcttttgcgattcatcatggacctgtgtaccaaaatgcgatgtttggagatgtttgtattgccgtaaggaattttttgagaagcaaccctagtgaaacagtatttatgcggataaaagaagaacatacagcagaaaacaatacaagatctttttcagatacatttgcagattataagtctcaatatagcgacttattttgggattggacaggtgataacccaagattaagtgaaataagaggaaaagttgttgttttacaaaatttttcaggtggtaaatttggtatcaattacaatacattgaatactcaagatcaatatcatttaaatacaaactgggatttatatgataaatggctattcgtcaaagaacatttgtatgccgctgacaactcttataaaagtggccgtaaacaagtatatctgaattacctaagtggatcaggtggttcatttccttattttgttgcaagtggacatagtagtccaggtacagatgctccacaattatctacaggtctaacaacaccagcatttgcaagctggtatccggattttccacggggaagttgttttataggaatttgcacaatttactttgaaggaacaaatattcttacaagtcagtggatagagaaaaatgattttaaatatataggaatcatagctgctgattttccaggaagaacattaatttccaatattattagtctgaataaacttcttagcttagaaattaaaaatggtggtacctatcaaattgtttccgctttaaataatagtagtgttatagatatgagtctgagtggagatcgaaatgttcacctatggtccaataacggtactcttaatcaagtatggaaattcgtgtatgattcaaatagattggcataccaaattaaaagtctatccgatgaaaatttagtactaacttgggcttattatagtagtaatcgagataatgtaattgttgcttctaatcaaaatagcgatgagcaatattggatacctgagcgcacaggcgcatatcattattttaaaaatctcatcaatccctcgggagcattagatgtaagcggatcaggaacaacaaacggaacgaatattttgtattggagttataacagagcaacgaatcaaaaattcaaactggaagaagtaaatatacctggaggtcaagctgaaggtgtacttttatatgcagatgctaattatgtagggaaatctgtactactaacaaatagtgtctcaaaccttagagacgttggtatgaatgatatagccagttctataaaatttattggtccttatcaagctactctatatgaacatgataattttacaggtgcggcttttactctcacatctaatgttgcaaatttaaaagatgttggcatgaatgatacagttagttctataaaaattacaaagacatctggaggccgagctacaggtatatatttatatgcagatgctaattatgtaggcagatctgtatggttaacatctaatgttgcaaatttaaaagatattggcatgaatgatacagtcagttctgtagaaattgttggcgcatatcaagccactttatatggggatgccaattatacagggaaggcttataatctcactcataatgttacaaatttaaaagatgttggcatgaatgatatagtcagttccataaaaatttttagtgtgtaa

SEQ ID NO: 4 - protein, 1217 aa, Bacillus thuringiensis
  REGION 1..1217: note = MISC_FEATURE - The amino acid sequence of a TIC7181 protein.
MDQKIIKMREAVNALFSNNHLKLNITDYNIDQTAYLVDSMSDDAYRQEKMMFLDQIKFAKRLSQKRNLLNHGDFEGSNWTGKNGWKRNNYVVVASDHPIFKGRYLHIPGATTAMSGAIIPTYVYQSIDESKLKPYTRYLVRGFVGKSQDLALLVSRYTKEVYKKINVPNDKDYDMTSHINREENLWHNRYIKDTSVQNSISMCKNPHEFTCHIDIGELDRKKGPGITIGFQIGTTDGMATLDNIEVIEAHPLTGYALARIEKRERKWKQKWLEHRIQIEKAVQTAQEVIRNLFTCPQQNQLNWMTTRNDIAHAETLIKEISYRYSQLSCGDFPILPEEAYDILQQLSTAVETAKALYTQRNVVNNGDFQAGLSNWHRTDGAEIQQIQNASSVLIITDWAANISQDMRVVEKGSYLLRVTAKKEDAGEGYITISDCAALIETLTFTTGESVESLTHSDIHSRLHKRYNKKHIKNHPSEEYEIESDLHLFNRAEQNGSLPSSYVTKTMEIFPETNRIRIEIGETGGTFIVESVELIRMEQMNETNNPDVDVQIVMNDTPATQFDPVSFTESTVRPRNAQYAYSHDSNIGYENPNWMADISGDTLFTDLSIPGTHNTMALYGGDITQCQTMSLNTQLHVGIRYLDIRCRHIENAFAIHHGPVYQNAMFGDVCIAVRNFLRSNPSETVFMRIKEEHTAENNTRSFSDTFADYKSQYSDLFWDWTGDNPRLSEIRGKVVVLQNFSGGKFGINYNTLNTQDQYHLNTNWDLYDKWLFVKEHLYAADNSYKSGRKQVYLNYLSGSGGSFPYFVASGHSSPGTDAPQLSTGLTTPAFASWYPDFPRGSCFIGICTIYFEGTNILTSQWIEKNDFKYIGIIAADFPGRTLISNIISLNKLLSLEIKNGGTYQIVSALNNSSVIDMSLSGDRNVHLWSNNGTLNQVWKFVYDSNRLAYQIKSLSDENLVLTWAYYSSNRDNVIVASNQNSDEQYWIPERTGAYHYFKNLINPSGALDVSGSGTTNGTNILYWSYNRATNQKFKLEEVNIPGGQAEGVLLYADANYVGKSVLLTNSVSNLRDVGMNDIASSIKFIGPYQATLYEHDNFTGAAFTLTSNVANLKDVGMNDTVSSIKITKTSGGRATGIYLYADANYVGRSVWLTSNVANLKDIGMNDTVSSVEIVGAYQATLYGDANYTGKAYNLTHNVTNLKDVGMNDIVSSIKIFSV

SEQ ID NO: 5 - DNA, 3654 bp, Bacillus thuringiensis
  misc_feature 1..3654: note = The native nucleotide sequence encoding TIC4904 toxin protein obtained from Bacillus thuringiensis species CFB007432.
  source 1..3654: mol_type = unassigned DNA
atggatcaaaagattataaaaatgcgagaagcagtcaatgccttgttttccaataatcagttaaaattgaatattactgattacaatatagatcagattgcataccttgttgatagtatgtctgatgacgcatatcgacaagaaaaaatgaggtttctcgatcaaatcaaatttgcaaagcgcttgagtcaaaaacgcaacctgttgaattatggagattttgaaggatccaattggccaggtaagaatggatggaaaagaaataattatgtagttgtcgcatcggatcatcctatatttaaaggccgatatttacacataccaagtgcaacaaccacgatgagtggcgcaatcattccgacttatgtatatcaacgtatagatgaatcgaagttaaaaccgtatacacgttatttggtacgagggtatgttggaaagagtcaagatttagcgttacttgtttcccggtataccaaagaagtgtacaagaaaatcaatgtaccaaatgatgaagattacgatatcacatcgcatataaatagggaagagaatctatggcacaatagatatataagaggcacccaggttcaaaattcaatctctatgtgcaacaatccacatgaatttacgtgtcatattgatataggggaactggatagaaagaaaggtcctggtataaccatcggttttcaaattggaacaacagatgggatggcaacattagataatatagaagtgatagaagcacatccgttaactggatcggccttagcacgtatcgaaaaacgtgaacgtaaatggaaacaaaaatggctagagaatcaaatacaaatcgaaaaggctgtgcaaacagtgcaagaggtgattcgaaatttatttacatgcccacaacaaaatcaattgaactggatgacaacccgaaacgacattgcacatgcagaaacattgataaaagagattccatatcggtatagtcaactttcttgtggagatttccccatactaccagaagaggcatatgacatccttcaacaactttcaactgcagttgaaaccgcaaaaacgttgtatacacagcgaaatgtggtgaagaatggggattttcaagctggattatcaaattggcataggacagatggtgcagagatacaacaaattcagaatacatcctctgttctggtaattacagactgggctgcgaatatttcacaagacatgcgtgtagttgaaaaaggtggatatctgttgcgcgtaacagcgaaaaaagaaaatccgggagaaggttatataactattagtgattgtgccgcattgacagaaacactgaaatttacagcgggggaatctgtagaaagtctgacacattctgatatttattcaaggctccataagcgctctgataaagaacaaataacaaaccatctttcaaaagaatatgaaatagaatcggatcctcatttattaaatagggcagaacaaaatggttctctcccttttagctatgtaaccaaaacaattgaaatttttccggaaaccaatcgagtacgcattgaaattggagaaacaggtggaacatttatagtggaaagtgtggaattgattcaaatggaacaggtaaacgaaacaaacaatccaactgtagatgttcaaattgtaatgaatgatacacccgctacaaaatttaatccagtttcttttacagaatcaacggtgagtcctagaactgttcattatgcgtattcacatgattcaagtataggttatgaaaaccctaactggatggatgatatttcaggtgatactttatttagtgatttatctctccctggtacacataatacaatggctctttatggaggagatattacacaatgccaaacgatgtcactgagtacgcaattacaagtaggaattcgttatttagatattcgctgtaggcacatagaaaatgtttttgctattcatcatggacctgtgtaccaaaatgcgatgtttggagatgtttgtattgccgtaaggaattttttgaaaagcaaccctagtgaaacagtatttatgcggattaaagaagaacatacagcagaaaacaatacaagatctttttcagatacatttgcagattataagtctcaatatagcgacttattttgggattggacaggtgataatccaagattaagtgaaataagaggaaaagttgttgttttgcaaaattttataggtgctaaatttggtatccattacgatacattgaataaacaagatcaatatcatttaaatacaaactgggatttatatgataaatggatattcgtcaaagaacatttgtatgccgctgacaactcttataaaagtggccgtaaacaagtatatctgaattacctaagtggatcaggtggttcatttccttattttgttgcaagtggacatagtagtccaggtacagatgctccacaattatctacaggtctaacaacaccagcatttgcaagctggtatccggattttccacggggaagttgttttataggaatttgcacaatttactttgaaggaacaaatattcttacaagtcagtggatagagaaaaatgattttaaatatataggaatcatagctgctgattttccaggaagaacattaatttccaatattattagtctgaataaacttcttagcttagaaattaaaaatggtggtacctatcaaattgtttccgctttaaataatagtagtgttatagatatgagtctgagtggagatcgaaatgttcacctatggtccaataacggtactcttaatcaagtatggaaattcgtgtatgattcaaatagattagcatatcaaattaaaagtctatccgatgaaaatttagtactaacttgggcttattatagtagtaatcgagataatgtaattgttgcttctaatcaaaatagcgatgagcaatattggatacctgagcgcacaggcgcatatcattattttaaaaatctcatcaatccctcgggagcattagatgtaagcggatcaggaacaacaaacggaacgaatattttgtattggagttataacagagcaacgaatcaaaaattcaaactggaagaagtaaatatacctggaggtcaagctgaaggtgtacttttatatgcagatgctaattatgtagggaaatctgtactactaacaaatagtgtctcaaaccttagagacgttggtatgaatgatatagccagttctataaaatttattggtccttatcaagctactctatatgaacatgataattttacaggtgcggtttttactcccacatctaatgttgcaaatttaaaagatgttggcatgaatgatacagttagttccataaaaattacaaagacatctggaggccgagctacaggtatatatttatatgcagatgctaattatgtaggcagatctgtatggttaacatcaaatgttgcaaatttaaaagatgttggcatgaatgatacagtcagttctgtagaaattgttggcgcgtatcaggccactttatatggggattccaattatacagggaaggcttataatctcactcataatgttgcaaatttaaaagatgttggcatgaatgatatagtcagttccataaaaatttttagtgtgtaa

SEQ ID NO: 6 - protein, 1217 aa, Bacillus thuringiensis
  REGION 1..1217: note = MISC_FEATURE - The amino acid sequence of a TIC4904 protein.
MDQKIIKMREAVNALFSNNQLKLNITDYNIDQIAYLVDSMSDDAYRQEKMRFLDQIKFAKRLSQKRNLLNYGDFEGSNWPGKNGWKRNNYVVVASDHPIFKGRYLHIPSATTTMSGAIIPTYVYQRIDESKLKPYTRYLVRGYVGKSQDLALLVSRYTKEVYKKINVPNDEDYDITSHINREENLWHNRYIRGTQVQNSISMCNNPHEFTCHIDIGELDRKKGPGITIGFQIGTTDGMATLDNIEVIEAHPLTGSALARIEKRERKWKQKWLENQIQIEKAVQTVQEVIRNLFTCPQQNQLNWMTTRNDIAHAETLIKEIPYRYSQLSCGDFPILPEEAYDILQQLSTAVETAKTLYTQRNVVKNGDFQAGLSNWHRTDGAEIQQIQNTSSVLVITDWAANISQDMRVVEKGGYLLRVTAKKENPGEGYITISDCAALTETLKFTAGESVESLTHSDIYSRLHKRSDKEQITNHLSKEYEIESDPHLLNRAEQNGSLPFSYVTKTIEIFPETNRVRIEIGETGGTFIVESVELIQMEQVNETNNPTVDVQIVMNDTPATKFNPVSFTESTVSPRTVHYAYSHDSSIGYENPNWMDDISGDTLFSDLSLPGTHNTMALYGGDITQCQTMSLSTQLQVGIRYLDIRCRHIENVFAIHHGPVYQNAMFGDVCIAVRNFLKSNPSETVFMRIKEEHTAENNTRSFSDTFADYKSQYSDLFWDWTGDNPRLSEIRGKVVVLQNFIGAKFGIHYDTLNKQDQYHLNTNWDLYDKWIFVKEHLYAADNSYKSGRKQVYLNYLSGSGGSFPYFVASGHSSPGTDAPQLSTGLTTPAFASWYPDFPRGSCFIGICTIYFEGTNILTSQWIEKNDFKYIGIIAADFPGRTLISNIISLNKLLSLEIKNGGTYQIVSALNNSSVIDMSLSGDRNVHLWSNNGTLNQVWKFVYDSNRLAYQIKSLSDENLVLTWAYYSSNRDNVIVASNQNSDEQYWIPERTGAYHYFKNLINPSGALDVSGSGTTNGTNILYWSYNRATNQKFKLEEVNIPGGQAEGVLLYADANYVGKSVLLTNSVSNLRDVGMNDIASSIKFIGPYQATLYEHDNFTGAVFTPTSNVANLKDVGMNDTVSSIKITKTSGGRATGIYLYADANYVGRSVWLTSNVANLKDVGMNDTVSSVEIVGAYQATLYGDSNYTGKAYNLTHNVANLKDVGMNDIVSSIKIFSV

SEQ ID NO: 7 - DNA, 3636 bp, Bacillus thuringiensis
  misc_feature 1..3636: note = The native nucleotide sequence encoding TIC6547 toxin protein obtained from Bacillus thuringiensis species CFB231019.
  source 1..3636: mol_type = unassigned DNA
atggatcaaaagattataaaaatgcgagaagcagtcaatgccttgttttccaataatcagttaaaattgaatattactgattacaatatagatcagattgcataccttgttgatagtatgtctgatgacgcatatcgacaagaaaaaatgaggtttctcgatcaaatcaaatttgcaaagcgcttgagtcaaaaacgcaacctgttgaattatggagattttgaaggatccaattggccaggtaagaatggatggaaaagaaataattatgtagttgtcgcatcggatcatcctatatttaaaggccgatatttacacataccaagtgcaacaaccacgatgagtggcgcaatcattccgacttatgtatatcaacgtatagatgaatcgaagttaaaaccgtatacacgttatttggtacgagggtatgttggaaagagtcaagatttagcgttacttgtttcccggtataccaaagaagtgtacaagaaaatcaatgtaccaaatgatgaggattacgatatcacatcgcatataaatagggaagagaatttatggcacaatagatatataagaggcacccaagttcaaaattcaatctctatgtgcaacaatccacatgaatttacgtgtcatattgatataggagaactggatagaaagaaaggtcctggtataaccatcggttttcaaattggaacaacagatgggatggcaacattagataatatagaagtgatagaagcacatccgttaactggatcggccttagcacgtatccaaaaacgtgaacgtaaatggaaacaaaaatggatagagaatcgaatgcaaatcgaaaaggctgtacaaacagcgcaagaggtgattcgaaatttatttacatgcccacaacaaaatcaattgaactggatgacaactcgaaacgacattacacatgcagaaacattgataaaagagattccatatcggtatagccaactttcttgtggagatttccccacactaccagaagaggcgtatgacatccttcaacaactttcaactgcagttgaaaccgcaaaagcgttatatgcacaacgaaatgtggtgaataatggggattttcaagctggattatcgaattggtatacgacagatggtgcagagatacaacaaatacagaattcgtcctctgttctagtaattaaagactgggctacaaatatttcacaggacatgcgtgtggttgaaaaaggtggctatctgctacgcgtaacagcgaaaaaagaagataccggagaaggttatataacaattagtgattgtgcagcattggtagaaaaattgacatttacaacgggggaagctgtagaaagtctggcacattctgatagtcgttcaaggctccataagcgctatgataaaaaatcagaaggatatgaaatagaatcggatcctcatttatttaatagggcgaaacaaaacggttctcttccttctagctatgtaaccaaaacgattgaaatctttccggaaaccaatcgagtacgcattgagattggagaaacaggtggaaagtttatggtggaaagtgtggaattgattcgaatggaacagatgaacgaaacaaataatccagctgtagatgttcaaactgtaatgaatgatacacctgctacacaatttgatccagtttcttttacagaatcaacggtgagtcccagaaatgctcagtatgcgtattctcatgatacaaatataggctatgaaaatcctaactggatggctgatatttcaggtgatactttatttagtgatttatctatccctggtacacataatacaatggctcttcatggaggagatattacacaatgtcaaacgatgtcactgaatacacaattacatgtaggaattcgttatttagatattcgctgtaggcatatcgataatgtttttgcgattcatcatgggcctgtgtaccaaaatacgatgtttggagatgtttgtatagccgtaagggattttttgaggaacaaccctagtgaaacagtatttatgcggataaaagaagaacatacaccagaaaataatacaagatctttttcggatacatttgcagattataagtctcaatatagcgacttattttggaattggacaggtgataacccaagattaagtgaaataagaggaaaagttgttgttttgcaaaacttttcaggggataggtttggtatctactacaatacactgaatacacaagatcaatatcatttagatacaaactgggatttatatgataaatggctatttgtaaaagagcatttgtataaagctgacgacgcttataaaagtggtggtaaacaagcatatctgaattatctaagtgggtcaggtggttcttttccttattttgttgcaagtggacatagtagtcctggtacagatgctccacaattatctacaggtctaacaacaccagcatttgcaagctggtatccggattttccacggggaagttgttttataggaatttgcacaatttactttgaaggaacaaatattcttacaagtcagtggatagagaaaaatgattttaaatatataggaatcatagctgctgattttccaggaagaacattaatttccaatattattagtttgaataaacttcttagcttagaaattaaaaatggtggtacctatcaaattgtttccgctttaaataatagtagtgttatagatatgagtctgagtggagatcgaaatgctcacctatggtccaataacggtactcctaatcaagtatggaaattcgtgtatgattcaaatagattagcataccaaattaaaagtttatccgatgaaaatttagtactaacttgggcttattatagtagtaatcgagataatgtaattgtcgcttctaatcaaaatagcgatgagcaatattggatacctgagcgcacaggcgcatatcattattttaaaaatctcatcaatccctcaggagcattagatgtaagcggatcaggaacaacaaacggaacgaatattttgtattggagttataacagagcaacgaatcaaaaattcaaactggaagaagtaaatatatctggaggtcaaactgaaggtgtacttttatatgcagaggctaattatgtagggaaatctgtactactaacaaatagtgtctccaaccttagagacgttggtatgaatgatatagctagttctataaaatttattggtccttatcaagctactctatatgaacatgatgattttacaggtgcggtttttactcccacatctaatgttgcaaatttaaaagatgttggcatgaatgatacagttagttctataaaaattacaaagacatctggaggccgagctacaggtatatatttatatgcagatgctaattatgtaggcagatctgtatggttaacatctaatgttgcaaatttaaaagatgttggcatgaatgatacagtcagttctgtagaaattgttggcgcgtatcaggccactttatatggggattccaattatacagggaaggcttataatctcactcataatgttgcaaatttaaaagatgttggcatgaatgatatagtcagttccataaaaatttttagtgtgtaa

SEQ ID NO: 8 - protein, 1211 aa, Bacillus thuringiensis
  REGION 1..1211: note = MISC_FEATURE - The amino acid sequence of a TIC6547 protein.
MDQKIIKMREAVNALFSNNQLKLNITDYNIDQIAYLVDSMSDDAYRQEKMRFLDQIKFAKRLSQKRNLLNYGDFEGSNWPGKNGWKRNNYVVVASDHPIFKGRYLHIPSATTTMSGAIIPTYVYQRIDESKLKPYTRYLVRGYVGKSQDLALLVSRYTKEVYKKINVPNDEDYDITSHINREENLWHNRYIRGTQVQNSISMCNNPHEFTCHIDIGELDRKKGPGITIGFQIGTTDGMATLDNIEVIEAHPLTGSALARIQKRERKWKQKWIENRMQIEKAVQTAQEVIRNLFTCPQQNQLNWMTTRNDITHAETLIKEIPYRYSQLSCGDFPTLPEEAYDILQQLSTAVETAKALYAQRNVVNNGDFQAGLSNWYTTDGAEIQQIQNSSSVLVIKDWATNISQDMRVVEKGGYLLRVTAKKEDTGEGYITISDCAALVEKLTFTTGEAVESLAHSDSRSRLHKRYDKKSEGYEIESDPHLFNRAKQNGSLPSSYVTKTIEIFPETNRVRIEIGETGGKFMVESVELIRMEQMNETNNPAVDVQTVMNDTPATQFDPVSFTESTVSPRNAQYAYSHDTNIGYENPNWMADISGDTLFSDLSIPGTHNTMALHGGDITQCQTMSLNTQLHVGIRYLDIRCRHIDNVFAIHHGPVYQNTMFGDVCIAVRDFLRNNPSETVFMRIKEEHTPENNTRSFSDTFADYKSQYSDLFWNWTGDNPRLSEIRGKVVVLQNFSGDRFGIYYNTLNTQDQYHLDTNWDLYDKWLFVKEHLYKADDAYKSGGKQAYLNYLSGSGGSFPYFVASGHSSPGTDAPQLSTGLTTPAFASWYPDFPRGSCFIGICTIYFEGTNILTSQWIEKNDFKYIGIIAADFPGRTLISNIISLNKLLSLEIKNGGTYQIVSALNNSSVIDMSLSGDRNAHLWSNNGTPNQVWKFVYDSNRLAYQIKSLSDENLVLTWAYYSSNRDNVIVASNQNSDEQYWIPERTGAYHYFKNLINPSGALDVSGSGTTNGTNILYWSYNRATNQKFKLEEVNISGGQTEGVLLYAEANYVGKSVLLTNSVSNLRDVGMNDIASSIKFIGPYQATLYEHDDFTGAVFTPTSNVANLKDVGMNDTVSSIKITKTSGGRATGIYLYADANYVGRSVWLTSNVANLKDVGMNDTVSSVEIVGAYQATLYGDSNYTGKAYNLTHNVANLKDVGMNDIVSSIKIFSV

SEQ ID NO: 9 - DNA, 3642 bp, Bacillus thuringiensis
  misc_feature 1..3642: note = The native nucleotide sequence encoding TIC4006 toxin protein obtained from Bacillus thuringiensis species WC12466.
  source 1..3642: mol_type = unassigned DNA
atgaatcaatatgttacaacagtgcaaaaggcagttaatgcattattttcaaataataccttacccttaaacattactgattataatatagaccagacagcatatcttgtagaacgtatatctaatgatagatattctaaagacaagatgatgttactcaatcaagtcaaatttgcgaaacgtttgagtcgagcgcgtaacttattgaaaggtggcgcttttgaattatcagataagaatagatggaagacaaacaattatgcgaatattttatcaggttctctcctatccaaaggccaatctttaaacattctaagcgcaagccctacagtaagtagtcaaattattccgactcatgtatatcaaagaatagatgaatcaaagttaaaaccatatacacgttatttagtaagagggttcgttgaaaagagtcgagatttagaactatttgtgctcagatataacaaagaggtgtataaaagaatcaatgtacccaagaatgaggattatcatatcacatcgcatttaaatgaagaagagaatccatggcacaataaatatatccaaaacactccggttcaaaattcaatctctatgcgcaagaattcacatgagtttacgtgtcatattgatataggggaactggatataaagaaaggacctggtataaccatcggttttcaaattagcacaacagatgggatggcaacattagataatatagaagtgatagaagcacatccgttaactggagacgatttaacacgtatccaaaggcgtgaacgtaaatggaaacaaaaatggctagagaatcaaatacaaatcgaaaaagctgcacaaacagcgaaagaggcgattaaaaatttatttacatgcccacaacaaaatcaattgacctggatgacaaccctaaacgacattatacaggcagaaaaattgatacaagagattccatattggtatagccgacttttaggtgaggatttccccatactaccagaagaggcatatgacacccttcaacaactttcaactgcagttgaaaccgcaaaattgttgtatgcacaacgaaatgtggtgaataatggggattttcaagctggattttcaaattggaatacgaccgatggtgcagagataaaacaaattcaggattcatcttctgttctagtaattacggactgggctgcaaatatttcacaggacatgcgtgtggttgaaaaaggtggctatctgctgcgcgtaacagcgaaaaaagaagatgccggagaaggttatataacaattagtgattgttccgtagtgatggaaaaattgacatttacaacaggggattctgtagagagtctggcacattctgatatttattcaaggatccataagcgctatgctaaaaaacaaataacaaatcatctttcagaaagatatgaaatagaatcgaatcctcatttaattaatagagcggaacaaaatgcttccctcccttctagctatgtaaccaaaacgattgaagtctttccggaaaccaatcgagtacgcgttgaaattggagaaacaggtggaacatttatcgtggaaagtgtcgaattgattcgaatggaacagatgaacgaaacaaacaatccagctgtagatattcaaactgtaatgaatgatacacccgctacacaatttgatccagtttcttttacagaatcaacggtgagtcccagaaatactcaatatgcatattctcatgattcaaatataggttatgaaaatcctaactggatggctgatatttcaggtgatactttatttagtgatttatctatccctggtacacataatacaatggctttttatggaggagatattacacaatgtcaaacgatgtcactgaatacgcaattacatgtaggaattcgttatttagatattcgctgtaggcatatcgaaaatatttttgcgattcatcatggaattgtgtaccaaaatgcgacgtttacagatgtttgtatagccgtaagagattttttgaggaacaaccctagtgagacagtatttatgcggataaaagaagaacatacagcagaaaataatacaagatcttttggggagacatttgcagactataagtctcaatatagcgacttattttggaattggacgggtgataacccaagattaagtgaaataagaggaaaagttgttgttttgcaaaatttttttggggataaatttggtatcgattacaatacactgaataaacaagatcaatatcatttaaatacaaactgggatttatatgataaatggctatttgtaaaagaacatttgtatgccgctgacgattcttataaaaatggtcgtaaacaagcatatctaaattatctaagcgggtcaggtggttcttttccttattttgttgcaagtggacacagtagtcctggtacaaatgcttcaaatctatctacagggctaacaacaccggcatttgaaagctggtatccggattttccacggggaagttgttttataggaatttgcacaatttattttgaaggaacaaatattcttacaagtgagtggatacagaaaagtgattttaaatatgtaggaatcatagctgctgattttccaggaagaacattaatttccaatattattagtctgaataatcttcttagtttagaaattaaaaatggtggtacctatcaaattgtttccgctttaaataatagtagtgttgtagatatgaatccaggagaccaaaatattcacttatggaacaataacggtactgctaatcaattatggaaattcgtatataattcaaatgaattagcataccaaattaaaagtttatctaatgaaaatttagtattaacctgggcttacaatagtagtaatccagataatgtaattgctgcttccaatcaaaataggtctgagcaatattggatacctgagcgtacgggagcatatcattattttaaaaatctaagcaatcgttcgggagcattagatgtaagcggctcagagacaaaaaacggaacaaacattctgtactggagttataaaaaagcaacaaatcaaaaattcaaactgacagaagtaaatgtatctggaggtcaagctgaaggtgtatatttatatgcagatgccaattatgtagggcaatctgtagggctaacaaatagtgtcgcagaccttagcgaagttggtatgaatgatatagctagttctataaaatttattggtccttatcaagctactctatatgagcatgctgattttaaaggtgcggtttttactcccacaactaatattgcaaatttaaaagatgttggcatgaatgatacaatcagctctataaaaattacaaagacatctggaggccgagctgcaggtatatatttatattcggatgccaattatgtgggaaggtctatatggttaacgtctaatgttgcaaatttaaaagatgttggcatgaatgatacaatcagttccgtagaaattgttggcgcatatggagtcactttatatggggatgccaattatacaggtaaggcttatgctctcacatctaatgttgcaaatttaaaagatgttggcatgaatgatatagtcagttctataaaaatttttagtgtataa

SEQ ID NO: 10 - protein, 1213 aa, Bacillus thuringiensis
  REGION 1..1213: note = MISC_FEATURE - The amino acid sequence of a TIC4006 protein.
MNQYVTTVQKAVNALFSNNTLPLNITDYNIDQTAYLVERISNDRYSKDKMMLLNQVKFAKRLSRARNLLKGGAFELSDKNRWKTNNYANILSGSLLSKGQSLNILSASPTVSSQIIPTHVYQRIDESKLKPYTRYLVRGFVEKSRDLELFVLRYNKEVYKRINVPKNEDYHITSHLNEEENPWHNKYIQNTPVQNSISMRKNSHEFTCHIDIGELDIKKGPGITIGFQISTTDGMATLDNIEVIEAHPLTGDDLTRIQRRERKWKQKWLENQIQIEKAAQTAKEAIKNLFTCPQQNQLTWMTTLNDIIQAEKLIQEIPYWYSRLLGEDFPILPEEAYDTLQQLSTAVETAKLLYAQRNVVNNGDFQAGFSNWNTTDGAEIKQIQDSSSVLVITDWAANISQDMRVVEKGGYLLRVTAKKEDAGEGYITISDCSVVMEKLTFTTGDSVESLAHSDIYSRIHKRYAKKQITNHLSERYEIESNPHLINRAEQNASLPSSYVTKTIEVFPETNRVRVEIGETGGTFIVESVELIRMEQMNETNNPAVDIQTVMNDTPATQFDPVSFTESTVSPRNTQYAYSHDSNIGYENPNWMADISGDTLFSDLSIPGTHNTMAFYGGDITQCQTMSLNTQLHVGIRYLDIRCRHIENIFAIHHGIVYQNATFTDVCIAVRDFLRNNPSETVFMRIKEEHTAENNTRSFGETFADYKSQYSDLFWNWTGDNPRLSEIRGKVVVLQNFFGDKFGIDYNTLNKQDQYHLNTNWDLYDKWLFVKEHLYAADDSYKNGRKQAYLNYLSGSGGSFPYFVASGHSSPGTNASNLSTGLTTPAFESWYPDFPRGSCFIGICTIYFEGTNILTSEWIQKSDFKYVGIIAADFPGRTLISNIISLNNLLSLEIKNGGTYQIVSALNNSSVVDMNPGDQNIHLWNNNGTANQLWKFVYNSNELAYQIKSLSNENLVLTWAYNSSNPDNVIAASNQNRSEQYWIPERTGAYHYFKNLSNRSGALDVSGSETKNGTNILYWSYKKATNQKFKLTEVNVSGGQAEGVYLYADANYVGQSVGLTNSVADLSEVGMNDIASSIKFIGPYQATLYEHADFKGAVFTPTTNIANLKDVGMNDTISSIKITKTSGGRAAGIYLYSDANYVGRSIWLTSNVANLKDVGMNDTISSVEIVGAYGVTLYGDANYTGKAYALTSNVANLKDVGMNDIVSSIKIFSV

SEQ ID NO: 11 - DNA, 3657 bp, synthetic construct
  misc_feature 1..3657: note = A synthetic coding sequence encoding a TIC4747PL pesticidal protein designed for expression in a plant cell wherein an additional alanine codon is inserted immediately following the initiating methionine codon.
  source 1..3657: mol_type = other DNA
atggctgaccagaagatcattaagatgcgggaggcggtgaacgccctgttctccaacaatcatcttaaactcaacataacggactacaacatcgaccaaacggcctacctggttgacagcatgagcgacgacgcctaccgccaggagaagatgatgttcctcgaccagataaagtttgcgaagcggttaagccagaagcgcaatctcctgaatcatggcgacttcgagggtagcaattggacgggcaagaacggatggaagcggaataactacgtggtcgtggcctccgaccatcccatcttcaagggccgctatctccacatccctggcgcgacaacggccatgtccggagccatcatcccgacctatgtgtaccagtccattgatgagagtaaactcaagccatacacacgctaccttgtgcgcgggttcgttggcaagagccaggacttagcgctgctggtgtctcggtacacgaaggaggtgtacaagaagatcaacgtaccgaacgacaaggactacgacatgacttctcacatcaaccgggaggagaacctgtggcataaccgctacatcaaggacacttcggttcagaactccatctccatgtgcaagaacccgcatgagttcacctgccacattgacatcggcgaactcgaccggaagaagggccctggcattacaatcggcttccagattggcacgacagacggcatggcaacactggacaacatcgaggtgatcgaggcccacccgctcaccggttacgcgctggcacggatcgagaagcgcgagcgcaagtggaagcagaagtggctggagcaccgaatccagatcgagaaggctgtgcagaccgcccaggaagtcatcaggaacctcttcacctgtccacagcagaaccagcttaactggatgaccacccggaacgacatcgcccacgccgagaccctcatcaaggagatttcctaccgatactcccaactatcctgcggtgacttcccgatcttacccgaggaggcgtacgacatactccagcagctttccacggcggtcgagacggcgaaggcactctacacacaacgtaacgtcgtcaacaatggcgacttccaggcgggcttgagtaattggcacagaaccgacggtgccgagatacagcaaatccagaacgcatcgtcggtgctcatcatcacggactgggccgccaacatctctcaagacatgagagtagtcgagaagggttcttatctcctaagggtcaccgctaagaaggaggacgctggcgaagggtacattaccataagcgactgcgccgccctcattgagacccttaccttcaccacgggcgaatcggttgagtccttaactcattccgacattcactcccgtctccacaagcgctacaacaagaaacacattaagaaccacccatccgaggagtacgaaatagagagcgacctccacctctttaaccgtgcggagcagaatggctcgttgccatcatcctacgtcactaagacgatggaaatcttcccggagaccaacagggtgcgaattgaaatcggcgagacgggcggaaccttcatcgtggagtctgtagaactcatccgcatggaacagatgaacgagacaaacaatcccgatgtggatgttcagatcgtgatgaatgacacgccagcaacccagtttgatccggtgtcgtttaccgagagcaccgtccggccgcgcaatgcccagtacgcctactctcatgattccaacatcgggtacgagaaccctaattggatggccgacatcagcggcgacactctgttcactgacttgtccatccctggtacgcataacactatggctctttacggcggcgacattacccaatgtcagacaatgtcgctgaacacccagttgcacgtgggaatccgctacctggacatccgctgccgccacatcgagaacgctttcgccatccaccacggcccggtgtaccagaacgccatgttcggcgatgtgtgcatagccgttcgcaactttctccggagtaacccgagtgagactgtgttcatgcgtatcaaggaggagcacactgctgagaacaacacaaggtctttctcagacacattcgccgattacaagtcacagtacagcgacctgttctgggactggactggcgacaatccgaggctcagcgagatcagaggcaaagtagtggtgctccagaacttctctggcgggaaattcgggataaactacaacaccctcaacacacaggatcagtaccacctaaataccaattgggatctctacgataaatggctgttcgtcaaggagcatctgtacgctgctgacaacagctacaagtccggtaggaagcaagtgtacttaaattacctgtcgggatctggcggttccttcccgtactttgtggcgtcaggccattcaagtccgggcactgacgcgccgcaactttctactggccttaccacgcccgccttcgcctcgtggtatcctgacttcccacgcggtagctgcttcatcggcatctgcactatctacttcgagggcaccaacattctgacaagccagtggatcgagaagaatgacttcaagtacattgggatcatcgcggccgactttccgggtcgcaccctcatctcaaacattatttccctgaacaaactcctgtccctggagattaagaacggcggcacttatcagatagtatccgcgctcaataacagctcggtcattgacatgagcctttcgggcgaccggaacgtgcacctgtggtcgaataacggcacactgaaccaggtgtggaagttcgtctatgactcaaaccggctggcataccagatcaagagtctttctgacgagaatttggtcctcacctgggcctattactcctccaacagagacaacgtcatagtggccagcaaccagaactcggacgagcagtattggattccagagcgtactggagcttaccattactttaagaacctcatcaacccgagtggcgcgctcgacgtcagtggttccggcaccactaacgggaccaacatactctactggtcgtacaacagggcgaccaaccagaaattcaagctagaggaggtgaacatccctggcggacaggcggaaggagttctgctgtacgccgatgccaactatgtgggcaagtcagtcctcttgacgaactcagtatccaacctccgcgacgtcggcatgaacgacatcgcaagttccatcaaattcatcgggccgtaccaggcgaccctctacgagcatgataactttaccggcgcggctttcaccctgacatcgaatgtggcgaatctcaaagacgtcggtatgaacgacacggtttcctccatcaagatcaccaagaccagtggcgggcgcgccacgggcatctatctgtacgcggacgccaactacgtcgggaggagcgtgtggctcactagcaacgtggccaacctcaaggacatcggaatgaacgataccgtgtcctccgtcgagatcgtgggtgcgtatcaggcgaccctctacggcgacgcaaattacacgggcaaggcgtacaacctcacgcacaatgtcacgaacctgaaagacgtgggcatgaacgacatcgtctccagtatcaagatcttcagcgtgtga

SEQ ID NO: 12 - protein, 1218 aa, synthetic construct
  REGION 1..1218: note = The amino acid sequence of TIC4747PL encoded by a synthetic coding sequence designed for expression in a plant cell, and wherein an additional alanine amino acid is inserted immediately following the initiating methionine.
MADQKIIKMREAVNALFSNNHLKLNITDYNIDQTAYLVDSMSDDAYRQEKMMFLDQIKFAKRLSQKRNLLNHGDFEGSNWTGKNGWKRNNYVVVASDHPIFKGRYLHIPGATTAMSGAIIPTYVYQSIDESKLKPYTRYLVRGFVGKSQDLALLVSRYTKEVYKKINVPNDKDYDMTSHINREENLWHNRYIKDTSVQNSISMCKNPHEFTCHIDIGELDRKKGPGITIGFQIGTTDGMATLDNIEVIEAHPLTGYALARIEKRERKWKQKWLEHRIQIEKAVQTAQEVIRNLFTCPQQNQLNWMTTRNDIAHAETLIKEISYRYSQLSCGDFPILPEEAYDILQQLSTAVETAKALYTQRNVVNNGDFQAGLSNWHRTDGAEIQQIQNASSVLIITDWAANISQDMRVVEKGSYLLRVTAKKEDAGEGYITISDCAALIETLTFTTGESVESLTHSDIHSRLHKRYNKKHIKNHPSEEYEIESDLHLFNRAEQNGSLPSSYVTKTMEIFPETNRVRIEIGETGGTFIVESVELIRMEQMNETNNPDVDVQIVMNDTPATQFDPVSFTESTVRPRNAQYAYSHDSNIGYENPNWMADISGDTLFTDLSIPGTHNTMALYGGDITQCQTMSLNTQLHVGIRYLDIRCRHIENAFAIHHGPVYQNAMFGDVCIAVRNFLRSNPSETVFMRIKEEHTAENNTRSFSDTFADYKSQYSDLFWDWTGDNPRLSEIRGKVVVLQNFSGGKFGINYNTLNTQDQYHLNTNWDLYDKWLFVKEHLYAADNSYKSGRKQVYLNYLSGSGGSFPYFVASGHSSPGTDAPQLSTGLTTPAFASWYPDFPRGSCFIGICTIYFEGTNILTSQWIEKNDFKYIGIIAADFPGRTLISNIISLNKLLSLEIKNGGTYQIVSALNNSSVIDMSLSGDRNVHLWSNNGTLNQVWKFVYDSNRLAYQIKSLSDENLVLTWAYYSSNRDNVIVASNQNSDEQYWIPERTGAYHYFKNLINPSGALDVSGSGTTNGTNILYWSYNRATNQKFKLEEVNIPGGQAEGVLLYADANYVGKSVLLTNSVSNLRDVGMNDIASSIKFIGPYQATLYEHDNFTGAAFTLTSNVANLKDVGMNDTVSSIKITKTSGGRATGIYLYADANYVGRSVWLTSNVANLKDIGMNDTVSSVEIVGAYQATLYGDANYTGKAYNLTHNVTNLKDVGMNDIVSSIKIFSV

SEQ ID NO: 13 - DNA, 3657 bp, synthetic construct
  misc_feature 1..3657: note = A synthetic coding sequence encoding a TIC7181PL pesticidal protein designed for expression in a plant cell wherein an additional alanine codon is inserted immediately following the initiating methionine codon.
  source 1..3657: mol_type = other DNA
atggctgaccagaagattatcaagatgcgtgaggcagtgaatgcgctgttcagcaacaaccatctcaagctgaacatcaccgactacaacatcgaccagaccgcgtacctagtggacagcatgtccgacgacgcctaccgtcaggagaagatgatgttcctggatcagatcaagtttgcaaagcgcctgtcccagaagcgcaacctgctgaaccacggcgacttcgagggttcgaactggacgggcaagaacggctggaagcggaacaactacgtggtggtggcgtccgaccacccgatcttcaaaggccgctacctccacatccctggcgcgaccacggccatgtcgggtgcaattatccctacctatgtgtaccagagcattgatgagagtaagctcaagccctacacacgctaccttgtgcgcggtttcgttggaaagagccaggatctcgccctgctcgtgagcagatacacgaaagaggtgtacaagaagatcaacgtgccgaacgacaaggactatgacatgacctcccacatcaaccgcgaggagaacctctggcacaatcgctacatcaaggacacctcggtccagaacagcatctccatgtgcaagaacccgcacgagttcacctgccacattgacattggtgagctcgaccgcaagaagggtccgggcattaccattggtttccagattgggactactgacggcatggcaacgctcgataacatcgaagttatcgaagcccatcctcttactggatacgcccttgctcgcatcgagaagcgtgaacgcaagtggaagcagaaatggctcgagcaccggatacagattgagaaggctgttcaaacagcacaggaggtgattcggaacctgttcacctgtccgcagcagaaccagctgaactggatgacaactcggaacgatatcgctcacgctgagaccctgatcaaggaaatctcttaccggtactctcagctgtcgtgcggcgactttcccatactcccggaggaggcgtatgacatcttgcagcaactttcgactgccgtcgagactgcaaaggccctctacactcagcggaacgtcgtgaacaacggcgacttccaggctggcttatcgaactggcaccgcactgacggagctgagatccagcagatccagaacgcatcatctgtcctgataataactgactgggcggcgaacatctcccaagacatgcgggtggttgagaagggcagctatctcctgagggtcacagccaagaaggaggacgccggtgaaggctacatcaccatctctgactgcgcggcactgatcgagaccctcactttcactacaggcgaaagcgtggagtccttaacgcactcagacattcacagtaggctacacaaacgctacaacaagaagcacatcaagaatcacccatccgaggagtacgagattgagtccgacctccacctgtttaaccgcgcggagcagaacggttcccttccctcgagctacgtgaccaagacgatggagatcttcccggagaccaaccggatccgcattgaaatcggagagacgggcggaacgttcatagtcgagagcgtcgaattgatccggatggagcagatgaacgagaccaacaacccggatgtcgacgtccaaatcgtgatgaacgacacgcccgcaacccagtttgacccggtcagcttcaccgagagcactgttcgcccgcgcaatgcccagtacgcctacagccacgatagtaacattggctacgagaacccgaactggatggccgacattagcggagacaccctgttcacggacctctcgatccctgggactcataacacaatggcactttacggtggcgatatcactcagtgccagacaatgagcctcaacactcagctccacgtgggcattcggtatttggacattcgttgtcgccacattgagaacgccttcgcgatccatcacgggcctgtgtaccagaacgccatgttcggcgacgtctgcatagcagtgcgcaactttcttaggtcaaacccatccgagactgtgttcatgcgtatcaaggaggagcacacggctgagaataacacccgttccttctctgatacgttcgccgattacaaatcccaatactccgacctcttctgggattggaccggtgacaacccaagactctccgagattcgcggcaaagttgttgtcttacagaatttctccggcggcaaattcgggataaactacaacaccctcaacacccaggaccagtatcatctgaacacaaattgggacttgtacgacaagtggctgttcgtcaaggaacacctctacgccgctgacaattcgtacaaatccggtcgcaaacaagtttatctgaactacctgtccggctcgggcggttcctttccttacttcgtcgctagcgggcacagcagtcctgggactgatgcgccgcaactatcgaccggtctcacaacgccagcgttcgccagctggtatccggatttcccgcgcggctcctgcttcatcgggatctgcaccatctatttcgagggcacgaacatcctgacaagccaatggatcgagaagaacgacttcaagtacattggaatcatagcggccgacttccctggacgtaccctcatctcgaacatcatctcccttaacaagcttctgtcactggagatcaagaatggcggcacctaccaaatcgttagcgcgcttaataacagcagcgtgatcgacatgtccctcagcggcgacagaaacgttcatctgtggtccaacaacggaacactcaatcaagtgtggaaattcgtgtacgacagcaaccgactggcataccagatcaagtccctgtcagacgagaatctcgtgctcacgtgggcttattacagctccaaccgtgataacgtcatcgtggctagtaaccagaactccgacgagcaatactggattccagaacgaacgggcgcataccactacttcaagaatctgatcaacccatccggagcccttgacgtgagtggcagcggtacgacgaacggaacgaacatcctctactggtcttacaatcgggccaccaaccagaagttcaagctcgaggaggtgaacattccgggaggtcaggccgagggcgtgctactgtacgccgacgcaaactacgtcggcaagtccgtcctactgaccaactccgtgagcaacctgagggacgtcggtatgaacgacattgcgtccagcatcaagttcattgggccctaccaggccacactgtacgagcacgacaatttcaccggcgcggcgttcactctcacctcaaacgtggccaacttgaaagacgtgggcatgaacgacacggtgtcctccattaagataacgaagacctctggtggtcgcgctacgggcatctacctctacgccgacgcgaactacgtcggtcggtcggtgtggctcacatccaacgtggctaacctcaaggacattggaatgaacgacacggtctccagcgtagagatcgtaggcgcctaccaagccaccctctacggcgatgcaaactacactggcaaggcgtacaacctaacccacaacgtgacgaacctcaaggacgttggtatgaacgacattgtgtccagtattaagatcttcagcgtctga

SEQ ID NO: 14 - protein, 1218 aa, synthetic construct
  REGION 1..1218: note = The amino acid sequence of TIC7181PL encoded by a synthetic coding sequence designed for expression in a plant cell, and wherein an additional alanine amino acid is inserted immediately following the initiating methionine.
MADQKIIKMREAVNALFSNNHLKLNITDYNIDQTAYLVDSMSDDAYRQEKMMFLDQIKFAKRLSQKRNLLNHGDFEGSNWTGKNGWKRNNYVVVASDHPIFKGRYLHIPGATTAMSGAIIPTYVYQSIDESKLKPYTRYLVRGFVGKSQDLALLVSRYTKEVYKKINVPNDKDYDMTSHINREENLWHNRYIKDTSVQNSISMCKNPHEFTCHIDIGELDRKKGPGITIGFQIGTTDGMATLDNIEVIEAHPLTGYALARIEKRERKWKQKWLEHRIQIEKAVQTAQEVIRNLFTCPQQNQLNWMTTRNDIAHAETLIKEISYRYSQLSCGDFPILPEEAYDILQQLSTAVETAKALYTQRNVVNNGDFQAGLSNWHRTDGAEIQQIQNASSVLIITDWAANISQDMRVVEKGSYLLRVTAKKEDAGEGYITISDCAALIETLTFTTGESVESLTHSDIHSRLHKRYNKKHIKNHPSEEYEIESDLHLFNRAEQNGSLPSSYVTKTMEIFPETNRIRIEIGETGGTFIVESVELIRMEQMNETNNPDVDVQIVMNDTPATQFDPVSFTESTVRPRNAQYAYSHDSNIGYENPNWMADISGDTLFTDLSIPGTHNTMALYGGDITQCQTMSLNTQLHVGIRYLDIRCRHIENAFAIHHGPVYQNAMFGDVCIAVRNFLRSNPSETVFMRIKEEHTAENNTRSFSDTFADYKSQYSDLFWDWTGDNPRLSEIRGKVVVLQNFSGGKFGINYNTLNTQDQYHLNTNWDLYDKWLFVKEHLYAADNSYKSGRKQVYLNYLSGSGGSFPYFVASGHSSPGTDAPQLSTGLTTPAFASWYPDFPRGSCFIGICTIYFEGTNILTSQWIEKNDFKYIGIIAADFPGRTLISNIISLNKLLSLEIKNGGTYQIVSALNNSSVIDMSLSGDRNVHLWSNNGTLNQVWKFVYDSNRLAYQIKSLSDENLVLTWAYYSSNRDNVIVASNQNSDEQYWIPERTGAYHYFKNLINPSGALDVSGSGTTNGTNILYWSYNRATNQKFKLEEVNIPGGQAEGVLLYADANYVGKSVLLTNSVSNLRDVGMNDIASSIKFIGPYQATLYEHDNFTGAAFTLTSNVANLKDVGMNDTVSSIKITKTSGGRATGIYLYADANYVGRSVWLTSNVANLKDIGMNDTVSSVEIVGAYQATLYGDANYTGKAYNLTHNVTNLKDVGMNDIVSSIKIFSV

SEQ ID NO: 15 - DNA, 3657 bp, synthetic construct
  misc_feature 1..3657: note = A synthetic coding sequence encoding a TIC4904PL pesticidal protein designed for expression in a plant cell wherein an additional alanine codon is inserted immediately following the initiating methionine codon.
  source 1..3657: mol_type = other DNA
atggctgaccagaagatcattaagatgcgcgaggcggtcaacgccctcttctcaaacaatcagctgaaactgaacattaccgactacaacatcgaccagattgcctacctggtggattcaatgtcggacgacgcctaccgccaggagaagatgcggttcctggaccagatcaagttcgccaaacgcctgtcccagaagcggaacttgctgaactacggcgacttcgaaggcagcaactggcccggcaagaatggctggaagcgcaacaattatgtggtcgtggcctccgatcacccgatcttcaagggccgctatctccacatcccgtcggccacaactaccatgagcggcgccatcattcccacgtacgtttaccaacgcattgatgagtctaagctaaagccgtacactcgctacttggtccgaggctacgttggcaagagtcaggatcttgccctcctggtttcacgatacaccaaggaggtgtacaagaagatcaatgtcccgaacgacgaggactacgacatcacttcccacatcaaccgggaggagaacctctggcacaaccgctacattcgcggcacgcaagtgcagaactctatttcaatgtgcaacaatcctcatgagtttacctgccacattgacatcggcgagctggacaggaagaagggccctgggatcacgataggcttccagattggcaccacggacgggatggcaacccttgacaacatcgaggtgatcgaagcgcaccctctgaccgggagcgcgctggcgagaatcgagaaacgggagcgcaagtggaagcagaagtggctcgagaaccagatacaaatcgagaaggcggtccagacagtccaggaagtcatccgtaacctctttacctgcccgcagcagaaccagttgaattggatgaccacaagaaatgacatcgcgcacgcggagaccctgatcaaggagattccctaccgctacagccaactctcctgcggcgacttcccgatcctcccggaggaggcgtacgacattctccagcagctcagcactgcagtcgagaccgcgaagactctgtacacccagaggaacgtggtcaagaacggcgacttccaagcgggtctttccaactggcatcgaaccgacggagcagagatacagcaaatccagaacactagcagcgtccttgtcatcacggactgggccgccaacatcagccaggacatgcgcgttgtcgagaagggcggatacctgctgcgcgtaacagctaagaaggagaaccctggcgagggttacatcacaatctcagactgcgcggcattaacagagacgttgaaattcaccgctggcgagtcagtcgagtcactcactcactccgacatctattcccggcttcacaagaggtcggacaaggaacagatcacgaaccatctgagcaaggagtacgagattgagagcgacccgcatctcctgaatcgggccgagcagaacggctcccttcctttctcctacgtgacgaagaccatcgagatcttccctgagaccaatcgcgtccggattgaaatcggcgagacaggcgggactttcattgtggagtcggtggagctcatccagatggagcaagtcaacgagaccaacaacccgaccgtggacgttcaaatagtcatgaatgacacaccggcaactaagttcaatccagtcagtttcacagagtcgacagtctcgccaaggacagtgcattacgcctactcacatgactcatccattggttacgagaacccgaactggatggacgatatcagcggagacaccctcttctcggatcttagcctcccagggactcacaatacgatggccctgtacggtggcgacattacccaatgccagacgatgagccttagcacacagctccaagtcggaatccgctacctggacattcgttgccgccacatcgagaacgtcttcgccattcatcacggcccggtctatcagaacgctatgttcggcgacgtctgcatagcagtccggaacttcttaaagtctaatccctcagagaccgtgttcatgcgcatcaaagaggaacacacagcggagaataacacacggtcattctccgatacctttgccgattacaaatcccaatactccgatctgttctgggattggacgggcgataacccgcgtctgagcgagatccgtgggaaagtggtggttctccagaacttcatcggtgcgaagttcggcattcactacgataccctcaataagcaagaccagtaccatctcaacactaactgggatctctacgacaagtggatcttcgtcaaggagcacctgtacgccgccgacaattcctacaagagcggtcggaagcaagtgtacctcaactatctgagtggctccggcggttccttcccgtacttcgtggcgtcaggccattccagccctggtacggacgcgccacagctctcaactggcctcaccacgcccgcgttcgcctcatggtatccagactttcctcgaggctcctgtttcattggtatttgcacaatctatttcgaaggcaccaacatcttaacttcccagtggatcgagaagaacgacttcaagtacatcggtatcatcgctgccgatttccctggtaggacgctcatctccaacatcatctcgctcaacaagctgctttcactggagatcaagaacggcgggacataccagatcgttagcgcgctgaataactccagcgtgatcgacatgagtctctctggcgaccgcaacgttcatctgtggtcaaacaacggcacgcttaaccaagtgtggaagttcgtctatgacagcaaccggcttgcctaccagatcaagagcctctcagacgagaatctggtcctgacctgggcgtactattccagcaaccgtgacaacgtcattgtcgcttccaaccagaactcagatgagcaatactggatacccgagcggactggcgcctaccactatttcaagaacctgattaacccgagcggcgcacttgacgtgagtggttcgggtaccacaaacggcaccaacattctctactggtcctacaaccgggccacgaatcagaagtttaagctcgaggaggtgaacatccctggcggtcaggccgaaggcgtcctgctttacgccgatgccaactacgtcggcaagtcggtgctcctcacaaacagcgtctccaaccttcgcgacgtcgggatgaatgacatcgcgtcttccatcaagtttatcggcccgtaccaggctaccctttacgagcatgataacttcacgggcgcggtgttcacgccgacctccaacgtggcgaacctcaaagacgtcggtatgaacgacacggtgtcatccatcaagattacgaagacctccggcggccgggccaccggcatctacctttacgccgatgccaattacgttggaaggtccgtatggctgacctcaaatgtggccaacctaaaggacgtcggtatgaacgacaccgtctccagcgtggagatcgtcggcgcgtatcaggcgacgctgtacggtgactccaactacacgggaaaggcctacaacctgacgcataacgtggccaaccttaaggacgtcggaatgaatgatatcgtctctagcatcaagatcttcagcgtttag

SEQ ID NO: 16 - protein, 1218 aa, synthetic construct
  REGION 1..1218: note = The amino acid sequence of TIC4904PL encoded by a synthetic coding sequence designed for expression in a plant cell, and wherein an additional alanine amino acid is inserted immediately following the initiating methionine.
MADQKIIKMREAVNALFSNNQLKLNITDYNIDQIAYLVDSMSDDAYRQEKMRFLDQIKFAKRLSQKRNLLNYGDFEGSNWPGKNGWKRNNYVVVASDHPIFKGRYLHIPSATTTMSGAIIPTYVYQRIDESKLKPYTRYLVRGYVGKSQDLALLVSRYTKEVYKKINVPNDEDYDITSHINREENLWHNRYIRGTQVQNSISMCNNPHEFTCHIDIGELDRKKGPGITIGFQIGTTDGMATLDNIEVIEAHPLTGSALARIEKRERKWKQKWLENQIQIEKAVQTVQEVIRNLFTCPQQNQLNWMTTRNDIAHAETLIKEIPYRYSQLSCGDFPILPEEAYDILQQLSTAVETAKTLYTQRNVVKNGDFQAGLSNWHRTDGAEIQQIQNTSSVLVITDWAANISQDMRVVEKGGYLLRVTAKKENPGEGYITISDCAALTETLKFTAGESVESLTHSDIYSRLHKRSDKEQITNHLSKEYEIESDPHLLNRAEQNGSLPFSYVTKTIEIFPETNRVRIEIGETGGTFIVESVELIQMEQVNETNNPTVDVQIVMNDTPATKFNPVSFTESTVSPRTVHYAYSHDSSIGYENPNWMDDISGDTLFSDLSLPGTHNTMALYGGDITQCQTMSLSTQLQVGIRYLDIRCRHIENVFAIHHGPVYQNAMFGDVCIAVRNFLKSNPSETVFMRIKEEHTAENNTRSFSDTFADYKSQYSDLFWDWTGDNPRLSEIRGKVVVLQNFIGAKFGIHYDTLNKQDQYHLNTNWDLYDKWIFVKEHLYAADNSYKSGRKQVYLNYLSGSGGSFPYFVASGHSSPGTDAPQLSTGLTTPAFASWYPDFPRGSCFIGICTIYFEGTNILTSQWIEKNDFKYIGIIAADFPGRTLISNIISLNKLLSLEIKNGGTYQIVSALNNSSVIDMSLSGDRNVHLWSNNGTLNQVWKFVYDSNRLAYQIKSLSDENLVLTWAYYSSNRDNVIVASNQNSDEQYWIPERTGAYHYFKNLINPSGALDVSGSGTTNGTNILYWSYNRATNQKFKLEEVNIPGGQAEGVLLYADANYVGKSVLLTNSVSNLRDVGMNDIASSIKFIGPYQATLYEHDNFTGAVFTPTSNVANLKDVGMNDTVSSIKITKTSGGRATGIYLYADANYVGRSVWLTSNVANLKDVGMNDTVSSVEIVGAYQATLYGDSNYTGKAYNLTHNVANLKDVGMNDIVSSIKIFSV

SEQ ID NO: 17 - DNA, 3639 bp, synthetic construct
  misc_feature 1..3639: note = A synthetic coding sequence encoding a TIC6547PL pesticidal protein designed for expression in a plant cell wherein an additional alanine codon is inserted immediately following the initiating methionine codon.
  source 1..3639: mol_type = other DNA
atggctgaccagaagatcatcaagatgcgtgaggccgtcaacgcgctcttctcaaacaaccagttgaagctgaacatcaccgactacaacattgatcagattgcttacctcgtcgactccatgtcggacgacgcgtacagacaggagaagatgcgattcctcgaccagataaagtttgccaagcggctgtctcagaaacgtaacctccttaactacggcgacttcgagggcagcaactggcccggcaagaacggctggaagcgcaacaactacgttgtggtcgcgtcagaccacccgatctttaagggccgctaccttcacattccgagcgccactaccaccatgtctggcgcgattatcccgacctacgtttaccagaggatcgacgagtcgaagctcaaaccctacactcggtatctggtgcgcgggtacgtagggaagtcccaggatctcgcgcttctggtctcccgctacaccaaggaagtgtacaagaagataaacgtcccgaatgatgaggactacgacatcacctcacacatcaaccgcgaggagaacctctggcataaccggtacatccgcggcactcaagtacagaactcgatctctatgtgcaacaacccgcacgagtttacatgccacattgacatcggcgagctggaccgcaagaagggcccagggatcaccatcggtttccagatcggaacaacggatggtatggcgaccttggacaacatcgaagtgatcgaggcccacccactaaccggctctgccctcgcgcgcatccagaaacgtgagcgtaagtggaagcagaagtggattgagaaccggatgcagatcgagaaggcagtccagacggctcaggaggtgatcaggaacctcttcacctgtccgcagcagaaccagcttaactggatgacaacaaggaacgacatcacccatgcagagacgctgataaaggagatcccgtaccgctacagccagctgtcgtgcggtgatttcccgactttgccggaggaggcgtacgacatcctacaacagctgagcaccgctgttgaaacagcgaaggctctttatgctcagaggaacgtggtgaacaacggcgatttccaggctggcctctcgaattggtacactacggacggcgcggagatccagcaaatccagaacagcagtagcgtcttagtcatcaaggactgggcaacgaacatctctcaggacatgcgcgtcgtggagaagggcggctacctcctgcgtgtcacagcgaagaaggaggatacgggtgaagggtacatcactatttccgattgcgctgccctggtggagaaacttaccttcactacgggcgaagcagttgaaagccttgcccactcggactcaaggtctcgcttgcacaaacggtacgataagaaatcagaaggttatgagatcgagagcgaccctcacctctttaaccgcgcgaagcagaacggctcactgccctcaagctacgtcaccaagacaatcgaaatcttcccggaaacgaaccgggttcgcattgagatcggtgagaccggtggcaagttcatggttgagtcagtagagctgatccgaatggagcagatgaacgagacgaacaacccggcagtggatgttcagacagttatgaacgatactcctgctacccagttcgacccggtctcgtttaccgagagcaccgtgtcgccacggaacgctcagtatgcgtactcgcacgacacaaacattggctacgagaacccgaattggatggcagatatcagcggtgatacgctcttcagcgacctgagcattcccggcacgcacaatacgatggctttgcatggcggcgatatcacccaatgtcagactatgtctctgaacacacagttgcacgtgggaatccgctacttggacatccgttgccgccacatcgacaacgtcttcgccatccatcacggcccggtgtaccagaacactatgttcggcgacgtctgcatagcagtgcgggacttcttgcgtaacaacccgagcgaaaccgtcttcatgcgtattaaggaggaacacactccggagaacaatacccgctctttcagtgacaccttcgccgactacaagtctcagtacagcgacctgttctggaactggacgggcgacaaccctcgcctatctgagatccgtggaaaggtcgtcgtgctgcagaacttctccggcgaccgcttcggcatctactacaatacgctgaacacccaggaccagtatcacctcgacactaactgggatctttatgacaagtggctcttcgtaaaggagcatctttacaaagccgatgatgcgtacaagtccggcggcaaacaggcttacctcaactatctaagtggctcgggcggctcgtttccctactttgtggcctcgggccactcatcgcctggtactgacgcgcctcagctcagcaccggcctcaccacgccggccttcgctagctggtatccagacttcccacgcggttcttgcttcatcgggatctgtacgatctacttcgagggcacaaacattctgaccagccagtggatcgagaagaatgatttcaagtacattggcataatcgcagccgacttcccaggccggacgctcatctcaaacatcatctccctaaacaagcttctcagcttggagatcaagaacggcggcacctatcagatagtcagcgcgctcaacaatagctctgtaatcgacatgtccctttccggagaccgtaatgcacacctatggtcaaacaacgggacgccaaaccaggtctggaagtttgtgtacgatagcaatcgcttagcataccagatcaagagcctcagcgacgagaacctcgtcctcacatgggcctattactcctccaaccgcgataacgtcatcgtcgcctcaaaccagaactcagacgagcagtattggattccagagcggactggcgcctatcactactttaagaacctcatcaatccgtcgggcgccctggatgtttccgggagcggcactaccaatgggaccaacatcttgtactggtcgtacaacagggccacgaaccagaagttcaagctcgaggaggtgaacatctccggaggccagactgagggcgtgcttctgtacgcggaggccaactatgttggcaagagtgtcctgctcacgaactccgtctccaatctgcgcgacgtgggcatgaacgacatcgcctctagcattaagttcatcggcccgtaccaagctacgctctacgagcatgacgacttcactggagccgtcttcacaccgacgagtaacgtggcgaacctcaaagacgttggaatgaacgatacggtcagctcgatcaagatcaccaagacgtcgggcggcagagccacgggcatctacctctacgccgacgccaactacgtgggccgctcggtgtggctgacctcgaacgtagccaatttgaaggacgtgggcatgaacgacaccgtgtcgagcgtggaaattgttggtgcctaccaggcgacactctacggcgacagcaactacaccggcaaggcgtacaacctgacacacaacgtggccaaccttaaagacgtgggcatgaacgacatcgtgtcttccatcaagatcttcagcgtgtga

SEQ ID NO: 18 - protein, 1212 aa, synthetic construct
  REGION 1..1212: note = The amino acid sequence of TIC6547PL encoded by a synthetic coding sequence designed for expression in a plant cell, and wherein an additional alanine amino acid is inserted immediately following the initiating methionine.
MADQKIIKMREAVNALFSNNQLKLNITDYNIDQIAYLVDSMSDDAYRQEKMRFLDQIKFAKRLSQKRNLLNYGDFEGSNWPGKNGWKRNNYVVVASDHPIFKGRYLHIPSATTTMSGAIIPTYVYQRIDESKLKPYTRYLVRGYVGKSQDLALLVSRYTKEVYKKINVPNDEDYDITSHINREENLWHNRYIRGTQVQNSISMCNNPHEFTCHIDIGELDRKKGPGITIGFQIGTTDGMATLDNIEVIEAHPLTGSALARIQKRERKWKQKWIENRMQIEKAVQTAQEVIRNLFTCPQQNQLNWMTTRNDITHAETLIKEIPYRYSQLSCGDFPTLPEEAYDILQQLSTAVETAKALYAQRNVVNNGDFQAGLSNWYTTDGAEIQQIQNSSSVLVIKDWATNISQDMRVVEKGGYLLRVTAKKEDTGEGYITISDCAALVEKLTFTTGEAVESLAHSDSRSRLHKRYDKKSEGYEIESDPHLFNRAKQNGSLPSSYVTKTIEIFPETNRVRIEIGETGGKFMVESVELIRMEQMNETNNPAVDVQTVMNDTPATQFDPVSFTESTVSPRNAQYAYSHDTNIGYENPNWMADISGDTLFSDLSIPGTHNTMALHGGDITQCQTMSLNTQLHVGIRYLDIRCRHIDNVFAIHHGPVYQNTMFGDVCIAVRDFLRNNPSETVFMRIKEEHTPENNTRSFSDTFADYKSQYSDLFWNWTGDNPRLSEIRGKVVVLQNFSGDRFGIYYNTLNTQDQYHLDTNWDLYDKWLFVKEHLYKADDAYKSGGKQAYLNYLSGSGGSFPYFVASGHSSPGTDAPQLSTGLTTPAFASWYPDFPRGSCFIGICTIYFEGTNILTSQWIEKNDFKYIGIIAADFPGRTLISNIISLNKLLSLEIKNGGTYQIVSALNNSSVIDMSLSGDRNAHLWSNNGTPNQVWKFVYDSNRLAYQIKSLSDENLVLTWAYYSSNRDNVIVASNQNSDEQYWIPERTGAYHYFKNLINPSGALDVSGSGTTNGTNILYWSYNRATNQKFKLEEVNISGGQTEGVLLYAEANYVGKSVLLTNSVSNLRDVGMNDIASSIKFIGPYQATLYEHDDFTGAVFTPTSNVANLKDVGMNDTVSSIKITKTSGGRATGIYLYADANYVGRSVWLTSNVANLKDVGMNDTVSSVEIVGAYQATLYGDSNYTGKAYNLTHNVANLKDVGMNDIVSSIKIFSV

SEQ ID NO: 19 - DNA, 3645 bp, synthetic construct
  misc_feature 1..3645: note = A synthetic coding sequence encoding a TIC4006PL pesticidal protein designed for expression in a plant cell wherein an additional alanine codon is inserted immediately following the initiating methionine codon.
  source 1..3645: mol_type = other DNA
atggctaaccagtatgtgacaacggtccagaaggcggtgaatgcgctgttctcgaataacacgcttccgctcaacatcaccgactacaacattgaccaaactgcctacctcgttgagcgcatctccaacgaccggtactccaaagacaagatgatgctcctcaaccaggtgaagtttgcgaagcgcctgagccgcgcccggaacttgctcaagggcggcgcgttcgaactgtccgacaagaaccgctggaagaccaacaactacgcaaacatcctgtccggttccctgctcagcaagggccagtccctcaacatcctgagcgcgagcccgaccgtgtcttcgcagattatccctacgcatgtgtaccagcgcatcgacgagagcaagctcaagccctacacacgctacctcgtgcgcggattcgttgagaagtcccgtgaccttgagctcttcgttcttcgctacaacaaggaggtgtacaagaggatcaacgtcccgaagaatgaggactaccacataacgagccacctgaacgaggaggagaatccgtggcacaacaagtacatccagaacactcccgtgcagaactcgatttccatgcgcaagaactcacacgagttcacatgccacatcgacattggtgaattggacatcaagaagggtccgggcatcacaatcggctttcagatctccacaacggacggtatggccactctcgataacatcgaagtcattgaggcccaccctcttaccggagatgaccttacccggattcaaagacgggagcggaagtggaagcagaagtggctcgagaaccagattcagattgagaaggccgctcaaacagcgaaagaggccatcaagaacctcttcacttgccctcaacagaaccagctcacgtggatgacaaccttaaacgatatcatccaggcggagaaacttattcaagaaatcccgtactggtactcacgtctgcttggtgaggacttccctatccttcctgaggaggcctacgacaccttgcagcagttatccaccgccgtggagaccgcgaagctgctgtacgctcagaggaacgtcgtcaacaatggcgacttccaggcgggcttctctaattggaacaccacggacggtgcggagataaagcaaatccaggactccagcagcgttctcgtgatcaccgactgggctgcgaacatctcacaggacatgcgcgtggtggagaagggaggttacctgctgagagttacagccaagaaggaggatgcgggcgagggctacataaccatctctgactgtagcgtagtgatggagaagctaaccttcactactggtgacagtgtcgagagcctggcgcactccgacatctactctcgcatccacaagcgctacgcgaagaagcagattacgaaccacctctcagagcgttatgagatcgagtcgaacccgcacttgattaaccgcgctgagcagaacgccagccttccatcaagctacgtgaccaagacgatcgaggtcttcccggagacaaatcgggtgcgcgtcgagataggtgagacaggcggcacattcattgtcgagagcgtcgagctgatccgaatggagcagatgaatgagaccaacaatcccgccgtcgacattcagaccgttatgaatgacacgcctgctacgcagttcgaccctgtttccttcactgagtcgacagtgtcgccaaggaacacgcaatacgcctattcacacgattcgaacatcggctatgagaaccctaactggatggctgacatctcaggcgataccttgttctcggatcttagcatcccaggcacacacaacacgatggccttctacggtggcgacatcacgcagtgccaaacgatgagcctcaacacccaacttcacgtgggcatccgctacctagacatccgctgcagacacattgagaacatcttcgccatccatcacggaattgtgtaccagaacgcgacctttactgacgtctgcatcgcagtcagggacttcctcaggaacaaccctagcgaaaccgtgtttatgaggatcaaggaggaacatacagcggagaacaacaccagatccttcggtgagacgttcgccgattacaagagccaatactccgacctcttctggaactggacaggcgataatccgaggctgtccgagatccgcgggaaggtggtggtacttcagaacttcttcggcgacaagttcgggatcgactacaacacgctgaacaaacaggatcaataccacctgaacactaactgggatctctacgacaagtggctcttcgtgaaggagcacctgtacgcggctgacgatagctacaagaacgggcgcaagcaagcgtacctcaactatcttagtggctccggaggttcattcccgtacttcgtcgcgtccggccattccagccctggtacgaatgcgtccaacctgtccaccggtctgacaacaccggcgtttgagtcttggtatccagatttccctcgcggctcttgcttcatagggatctgcaccatctactttgagggcacaaacatcctgacatcagagtggattcagaagtcggacttcaaatacgtgggcatcattgccgcggacttcccagggcgcaccctgatctccaacataatctcccttaataaccttctttcgctcgagattaagaacggcgggacataccagatcgtgtcggctcttaacaattcgagcgtggtggacatgaatcccggcgaccagaacatccacctgtggaacaataacggtacggctaaccagctgtggaagttcgtttacaacagcaacgagcttgcctatcagatcaagtctctctcaaacgagaacctcgttcttacgtgggcgtacaactcctccaacccggacaacgtgatcgcagcctcgaaccagaaccgttctgaacagtactggatccctgagcgcaccggtgcctaccactacttcaagaacctttcgaaccgctcgggagccttggatgtcagtgggtcggaaacgaagaacgggacgaacatcctgtactggtcctacaagaaggctactaatcagaagttcaagctcaccgaggtgaacgtttctggcggccaagccgaaggcgtgtacctgtacgctgacgcgaactacgtcggccagtctgtgggcctgaccaactccgtggccgacttgtcggaggtgggcatgaacgatatcgcttcttcgatcaagttcatcgggccctaccaggccacactctacgagcacgctgacttcaagggtgcggtcttcactccgaccaccaacatcgccaatctcaaggacgtgggcatgaacgacaccatctcgagcatcaagatcactaagacctccggcggaagggcggccggcatctacctctactccgacgccaactacgtcgggaggtctatctggctgacgagcaacgtcgcgaatcttaaagacgtgggcatgaacgataccatctcttccgtcgagatagtcggcgcgtacggcgtgaccctctacggcgacgcgaactacacgggcaaggcctacgcacttacctccaacgtggctaacctcaaggatgtcgggatgaacgacatcgtttcgagcatcaagatcttctcagtttag

SEQ ID NO: 20 - protein, 1214 aa, synthetic construct
  REGION 1..1214: note = The amino acid sequence of TIC4006PL encoded by a synthetic coding sequence designed for expression in a plant cell, and wherein an additional alanine amino acid is inserted immediately following the initiating methionine.
MANQYVTTVQKAVNALFSNNTLPLNITDYNIDQTAYLVERISNDRYSKDKMMLLNQVKFAKRLSRARNLLKGGAFELSDKNRWKTNNYANILSGSLLSKGQSLNILSASPTVSSQIIPTHVYQRIDESKLKPYTRYLVRGFVEKSRDLELFVLRYNKEVYKRINVPKNEDYHITSHLNEEENPWHNKYIQNTPVQNSISMRKNSHEFTCHIDIGELDIKKGPGITIGFQISTTDGMATLDNIEVIEAHPLTGDDLTRIQRRERKWKQKWLENQIQIEKAAQTAKEAIKNLFTCPQQNQLTWMTTLNDIIQAEKLIQEIPYWYSRLLGEDFPILPEEAYDTLQQLSTAVETAKLLYAQRNVVNNGDFQAGFSNWNTTDGAEIKQIQDSSSVLVITDWAANISQDMRVVEKGGYLLRVTAKKEDAGEGYITISDCSVVMEKLTFTTGDSVESLAHSDIYSRIHKRYAKKQITNHLSERYEIESNPHLINRAEQNASLPSSYVTKTIEVFPETNRVRVEIGETGGTFIVESVELIRMEQMNETNNPAVDIQTVMNDTPATQFDPVSFTESTVSPRNTQYAYSHDSNIGYENPNWMADISGDTLFSDLSIPGTHNTMAFYGGDITQCQTMSLNTQLHVGIRYLDIRCRHIENIFAIHHGIVYQNATFTDVCIAVRDFLRNNPSETVFMRIKEEHTAENNTRSFGETFADYKSQYSDLFWNWTGDNPRLSEIRGKVVVLQNFFGDKFGIDYNTLNKQDQYHLNTNWDLYDKWLFVKEHLYAADDSYKNGRKQAYLNYLSGSGGSFPYFVASGHSSPGTNASNLSTGLTTPAFESWYPDFPRGSCFIGICTIYFEGTNILTSEWIQKSDFKYVGIIAADFPGRTLISNIISLNNLLSLEIKNGGTYQIVSALNNSSVVDMNPGDQNIHLWNNNGTANQLWKFVYNSNELAYQIKSLSNENLVLTWAYNSSNPDNVIAASNQNRSEQYWIPERTGAYHYFKNLSNRSGALDVSGSETKNGTNILYWSYKKATNQKFKLTEVNVSGGQAEGVYLYADANYVGQSVGLTNSVADLSEVGMNDIASSIKFIGPYQATLYEHADFKGAVFTPTTNIANLKDVGMNDTISSIKITKTSGGRAAGIYLYSDANYVGRSIWLTSNVANLKDVGMNDTISSVEIVGAYGVTLYGDANYTGKAYALTSNVANLKDVGMNDIVSSIKIFSV

SEQ ID NO: 21 - DNA, 3678 bp, synthetic construct
  misc_feature 1..3678: note = A nucleotide sequence encoding TIC4747_His comprised of a histidine tag coding sequence operably linked 3' to the TIC4747 coding sequence.
  source 1..3678: mol_type = other DNA
atggatcaaaagattataaaaatgcgagaagcagtcaatgccttgttttccaataatcatttaaaattgaatattactgattacaatatagatcagactgcataccttgttgatagtatgtctgatgacgcatatcgacaagaaaaaatgatgtttctcgatcaaatcaaatttgcaaagcgcttgagccaaaaacgcaacctgttgaatcatggagattttgaaggatccaattggacaggtaagaatggatggaaaagaaataattatgtagttgtcgcatcggatcatcctatatttaaaggccgatatttacacataccaggtgcaacaaccgcgatgagtggcgcaatcattccgacttatgtatatcaaagtatagatgaatcgaagttaaaaccgtatacacgttatttggtacgagggtttgttggaaagagtcaagatttagcgttacttgtttcccggtataccaaagaagtgtacaagaaaatcaatgtaccaaatgataaagactacgatatgacatcgcatataaatagggaagagaatctatggcacaatagatatataaaagacacttcggttcaaaattcaatctctatgtgcaaaaatccacatgaatttacgtgtcatattgatataggggaactggatagaaagaaaggtcctggtataaccatcggttttcaaattggaacaacagatgggatggcaacattagataatatagaagtgatagaagcacatccgttaaccggatacgccttagcacgtatcgaaaaacgtgaacgtaaatggaaacaaaaatggctagagcatcgaatacaaatcgaaaaggctgtgcaaacagcgcaagaggtgattcgaaatttatttacatgcccacaacaaaatcaattgaactggatgacaacccgaaacgacattgcacatgcagaaacattgataaaagagatttcatatcggtatagccaactttcttgtggagatttccccatactaccagaagaggcgtatgacatccttcaacaactttcaactgcagttgaaaccgcaaaagcgttgtatacacaacgaaacgtggtgaataatggggattttcaagctggattatcgaattggcataggacagatggtgcagagatacaacaaattcagaatgcatcctctgttctaataattacagactgggctgcgaatatttcacaagacatgcgtgtagttgaaaaaggtagctatctgttgcgcgtaacagcgaaaaaagaagatgccggagaaggttatattacaattagtgattgtgccgcattgatagaaacattgacatttacaacgggggaatctgtggaaagtctgacacattctgatattcattcaaggctccataaacgctataataaaaaacacataaaaaaccatccttcagaagaatatgaaatagaatcggatcttcatttatttaatagggcggaacaaaacggttctctcccctctagctatgtaaccaaaacgatggaaatctttccggaaaccaatcgagtacgcattgaaattggagaaacaggtggaacatttatagtggaaagtgtggaattaattcgaatggaacagatgaacgaaacaaacaatccagatgtagatgttcaaattgtaatgaatgatacacccgctacacaatttgatccagtttcttttacagaatccacggtgaggcccagaaatgctcagtatgcatattctcatgattcaaatataggttatgaaaatcctaactggatggctgatatttcaggtgatactttatttactgatttatctatccctggtacacataatacaatggctctttatggaggagatattacacaatgtcaaacgatgtcactgaatacgcaattacatgtaggaattcgttatttagatattcgctgtaggcacatagaaaatgcttttgcgattcatcatggacctgtgtaccaaaatgcgatgtttggagatgtttgtattgccgtaaggaattttttgagaagcaaccctagtgaaacagtatttatgcggataaaagaagaacatacagcagaaaacaatacaagatctttttcagatacatttgcagattataagtctcaatatagcgacttattttgggattggacaggtgataacccaagattaagtgaaataagaggaaaagttgttgttttacaaaatttttcaggtggtaaatttggtatcaattacaatacattgaatactcaagatcaatatcatttaaatacaaactgggatttatatgataaatggctattcgtcaaagaacatttgtatgccgctgacaactcttataaaagtggccgtaaacaagtatatctgaattacctaagtggatcaggtggttcatttccttattttgttgcaagtggacatagtagtccaggtacagatgctccacaattatctacaggtctaacaacaccagcatttgcaagctggtatccggattttccacggggaagttgttttataggaatttgcacaatttactttgaaggaacaaatattcttacaagtcagtggatagagaaaaatgattttaaatatataggaatcatagctgctgattttccaggaagaacattaatttccaatattattagtctgaataaacttcttagcttagaaattaaaaatggtggtacctatcaaattgtttccgctttaaataatagtagtgttatagatatgagtctgagtggagatcgaaatgttcacctatggtccaataacggtactcttaatcaagtatggaaattcgtgtatgattcaaatagattggcataccaaattaaaagtctatccgatgaaaatttagtactaacttgggcttattatagtagtaatcgagataatgtaattgttgcttctaatcaaaatagcgatgagcaatattggatacctgagcgcacaggcgcatatcattattttaaaaatctcatcaatccctcgggagcattagatgtaagcggatcaggaacaacaaacggaacgaatattttgtattggagttataacagagcaacgaatcaaaaattcaaactggaagaagtaaatatacctggaggtcaagctgaaggtgtacttttatatgcagatgctaattatgtagggaaatctgtactactaacaaatagtgtctcaaaccttagagacgttggtatgaatgatatagccagttctataaaatttattggtccttatcaagctactctatatgaacatgataattttacaggtgcggcttttactctcacatctaatgttgcaaatttaaaagatgttggcatgaatgatacagttagttctataaaaattacaaagacatctggaggccgagctacaggtatatatttatatgcagatgctaattatgtaggcagatctgtatggttaacatctaatgttgcaaatttaaaagatattggcatgaatgatacagtcagttctgtagaaattgttggcgcatatcaagccactttatatggggatgccaattatacagggaaggcttataatctcactcataatgttacaaatttaaaagatgttggcatgaatgatatagtcagttccataaaaatttttagtgtgcaccaccatcacgctcaccatcactga

SEQ ID NO: 22 - protein, 1225 aa, synthetic construct
  REGION 1..1225: note = The amino acid sequence of TIC4747_His.
MDQKIIKMREAVNALFSNNHLKLNITDYNIDQTAYLVDSMSDDAYRQEKMMFLDQIKFAKRLSQKRNLLNHGDFEGSNWTGKNGWKRNNYVVVASDHPIFKGRYLHIPGATTAMSGAIIPTYVYQSIDESKLKPYTRYLVRGFVGKSQDLALLVSRYTKEVYKKINVPNDKDYDMTSHINREENLWHNRYIKDTSVQNSISMCKNPHEFTCHIDIGELDRKKGPGITIGFQIGTTDGMATLDNIEVIEAHPLTGYALARIEKRERKWKQKWLEHRIQIEKAVQTAQEVIRNLFTCPQQNQLNWMTTRNDIAHAETLIKEISYRYSQLSCGDFPILPEEAYDILQQLSTAVETAKALYTQRNVVNNGDFQAGLSNWHRTDGAEIQQIQNASSVLIITDWAANISQDMRVVEKGSYLLRVTAKKEDAGEGYITISDCAALIETLTFTTGESVESLTHSDIHSRLHKRYNKKHIKNHPSEEYEIESDLHLFNRAEQNGSLPSSYVTKTMEIFPETNRVRIEIGETGGTFIVESVELIRMEQMNETNNPDVDVQIVMNDTPATQFDPVSFTESTVRPRNAQYAYSHDSNIGYENPNWMADISGDTLFTDLSIPGTHNTMALYGGDITQCQTMSLNTQLHVGIRYLDIRCRHIENAFAIHHGPVYQNAMFGDVCIAVRNFLRSNPSETVFMRIKEEHTAENNTRSFSDTFADYKSQYSDLFWDWTGDNPRLSEIRGKVVVLQNFSGGKFGINYNTLNTQDQYHLNTNWDLYDKWLFVKEHLYAADNSYKSGRKQVYLNYLSGSGGSFPYFVASGHSSPGTDAPQLSTGLTTPAFASWYPDFPRGSCFIGICTIYFEGTNILTSQWIEKNDFKYIGIIAADFPGRTLISNIISLNKLLSLEIKNGGTYQIVSALNNSSVIDMSLSGDRNVHLWSNNGTLNQVWKFVYDSNRLAYQIKSLSDENLVLTWAYYSSNRDNVIVASNQNSDEQYWIPERTGAYHYFKNLINPSGALDVSGSGTTNGTNILYWSYNRATNQKFKLEEVNIPGGQAEGVLLYADANYVGKSVLLTNSVSNLRDVGMNDIASSIKFIGPYQATLYEHDNFTGAAFTLTSNVANLKDVGMNDTVSSIKITKTSGGRATGIYLYADANYVGRSVWLTSNVANLKDIGMNDTVSSVEIVGAYQATLYGDANYTGKAYNLTHNVTNLKDVGMNDIVSSIKIFSVHHHHAHHH

SEQ ID NO: 23 - DNA, 3714 bp, synthetic construct
  misc_feature 1..3714: note = A nucleotide sequence encoding TIC4904_His comprised of a histidine tag coding sequence operably linked 5' to the TIC4904 coding sequence.
  source 1..3714: mol_type = other DNA
atgcatcaccatcaccatcaccatcaccatcacggtaccgagaccgtccgcttccaatccatggatcaaaagattataaaaatgcgagaagcagtcaatgccttgttttccaataatcagttaaaattgaatattactgattacaatatagatcagattgcataccttgttgatagtatgtctgatgacgcatatcgacaagaaaaaatgaggtttctcgatcaaatcaaatttgcaaagcgcttgagtcaaaaacgcaacctgttgaattatggagattttgaaggatccaattggccaggtaagaatggatggaaaagaaataattatgtagttgtcgcatcggatcatcctatatttaaaggccgatatttacacataccaagtgcaacaaccacgatgagtggcgcaatcattccgacttatgtatatcaacgtatagatgaatcgaagttaaaaccgtatacacgttatttggtacgagggtatgttggaaagagtcaagatttagcgttacttgtttcccggtataccaaagaagtgtacaagaaaatcaatgtaccaaatgatgaagattacgatatcacatcgcatataaatagggaagagaatctatggcacaatagatatataagaggcacccaggttcaaaattcaatctctatgtgcaacaatccacatgaatttacgtgtcatattgatataggggaactggatagaaagaaaggtcctggtataaccatcggttttcaaattggaacaacagatgggatggcaacattagataatatagaagtgatagaagcacatccgttaactggatcggccttagcacgtatcgaaaaacgtgaacgtaaatggaaacaaaaatggctagagaatcaaatacaaatcgaaaaggctgtgcaaacagtgcaagaggtgattcgaaatttatttacatgcccacaacaaaatcaattgaactggatgacaacccgaaacgacattgcacatgcagaaacattgataaaagagattccatatcggtatagtcaactttcttgtggagatttccccatactaccagaagaggcatatgacatccttcaacaactttcaactgcagttgaaaccgcaaaaacgttgtatacacagcgaaatgtggtgaagaatggggattttcaagctggattatcaaattggcataggacagatggtgcagagatacaacaaattcagaatacatcctctgttctggtaattacagactgggctgcgaatatttcacaagacatgcgtgtagttgaaaaaggtggatatctgttgcgcgtaacagcgaaaaaagaaaatccgggagaaggttatataactattagtgattgtgccgcattgacagaaacactgaaatttacagcgggggaatctgtagaaagtctgacacattctgatatttattcaaggctccataagcgctctgataaagaacaaataacaaaccatctttcaaaagaatatgaaatagaatcggatcctcatttattaaatagggcagaacaaaatggttctctcccttttagctatgtaaccaaaacaattgaaatttttccggaaaccaatcgagtacgcattgaaattggagaaacaggtggaacatttatagtggaaagtgtggaattgattcaaatggaacaggtaaacgaaacaaacaatccaactgtagatgttcaaattgtaatgaatgatacacccgctacaaaatttaatccagtttcttttacagaatcaacggtgagtcctagaactgttcattatgcgtattcacatgattcaagtataggttatgaaaaccctaactggatggatgatatttcaggtgatactttatttagtgatttatctctccctggtacacataatacaatggctctttatggaggagatattacacaatgccaaacgatgtcactgagtacgcaattacaagtaggaattcgttatttagatattcgctgtaggcacatagaaaatgtttttgctattcatcatggacctgtgtaccaaaatgcgatgtttggagatgtttgtattgccgtaaggaattttttgaaaagcaaccctagtgaaacagtatttatgcggattaaagaagaacatacagcagaaaacaatacaagatctttttcagatacatttgcagattataagtctcaatatagcgacttattttgggattggacaggtgataatccaagattaagtgaaataagaggaaaagttgttgttttgcaaaattttataggtgctaaatttggtatccattacgatacattgaataaacaagatcaatatcatttaaatacaaactgggatttatatgataaatggatattcgtcaaagaacatttgtatgccgctgacaactcttataaaagtggccgtaaacaagtatatctgaattacctaagtggatcaggtggttcatttccttattttgttgcaagtggacatagtagtccaggtacagatgctccacaattatctacaggtctaacaacaccagcatttgcaagctggtatccggattttccacggggaagttgttttataggaatttgcacaatttactttgaaggaacaaatattcttacaagtcagtggatagagaaaaatgattttaaatatataggaatcatagctgctgattttccaggaagaacattaatttccaatattattagtctgaataaacttcttagcttagaaattaaaaatggtggtacctatcaaattgtttccgctttaaataatagtagtgttatagatatgagtctgagtggagatcgaaatgttcacctatggtccaataacggtactcttaatcaagtatggaaattcgtgtatgattcaaatagattagcatatcaaattaaaagtctatccgatgaaaatttagtactaacttgggcttattatagtagtaatcgagataatgtaattgttgcttctaatcaaaatagcgatgagcaatattggatacctgagcgcacaggcgcatatcattattttaaaaatctcatcaatccctcgggagcattagatgtaagcggatcaggaacaacaaacggaacgaatattttgtattggagttataacagagcaacgaatcaaaaattcaaactggaagaagtaaatatacctggaggtcaagctgaaggtgtacttttatatgcagatgctaattatgtagggaaatctgtactactaacaaatagtgtctcaaaccttagagacgttggtatgaatgatatagccagttctataaaatttattggtccttatcaagctactctatatgaacatgataattttacaggtgcggtttttactcccacatctaatgttgcaaatttaaaagatgttggcatgaatgatacagttagttccataaaaattacaaagacatctggaggccgagctacaggtatatatttatatgcagatgctaattatgtaggcagatctgtatggttaacatcaaatgttgcaaatttaaaagatgttggcatgaatgatacagtcagttctgtagaaattgttggcgcgtatcaggccactttatatggggattccaattatacagggaaggcttataatctcactcataatgttgcaaatttaaaagatgttggcatgaatgatatagtcagttccataaaaatttttagtgtgtaa

SEQ ID NO: 24 - protein, 1237 aa, synthetic construct
  REGION 1..1237: note = The amino acid sequence of TIC4904_His.
MHHHHHHHHHHGTETVRFQSMDQKIIKMREAVNALFSNNQLKLNITDYNIDQIAYLVDSMSDDAYRQEKMRFLDQIKFAKRLSQKRNLLNYGDFEGSNWPGKNGWKRNNYVVVASDHPIFKGRYLHIPSATTTMSGAIIPTYVYQRIDESKLKPYTRYLVRGYVGKSQDLALLVSRYTKEVYKKINVPNDEDYDITSHINREENLWHNRYIRGTQVQNSISMCNNPHEFTCHIDIGELDRKKGPGITIGFQIGTTDGMATLDNIEVIEAHPLTGSALARIEKRERKWKQKWLENQIQIEKAVQTVQEVIRNLFTCPQQNQLNWMTTRNDIAHAETLIKEIPYRYSQLSCGDFPILPEEAYDILQQLSTAVETAKTLYTQRNVVKNGDFQAGLSNWHRTDGAEIQQIQNTSSVLVITDWAANISQDMRVVEKGGYLLRVTAKKENPGEGYITISDCAALTETLKFTAGESVESLTHSDIYSRLHKRSDKEQITNHLSKEYEIESDPHLLNRAEQNGSLPFSYVTKTIEIFPETNRVRIEIGETGGTFIVESVELIQMEQVNETNNPTVDVQIVMNDTPATKFNPVSFTESTVSPRTVHYAYSHDSSIGYENPNWMDDISGDTLFSDLSLPGTHNTMALYGGDITQCQTMSLSTQLQVGIRYLDIRCRHIENVFAIHHGPVYQNAMFGDVCIAVRNFLKSNPSETVFMRIKEEHTAENNTRSFSDTFADYKSQYSDLFWDWTGDNPRLSEIRGKVVVLQNFIGAKFGIHYDTLNKQDQYHLNTNWDLYDKWIFVKEHLYAADNSYKSGRKQVYLNYLSGSGGSFPYFVASGHSSPGTDAPQLSTGLTTPAFASWYPDFPRGSCFIGICTIYFEGTNILTSQWIEKNDFKYIGIIAADFPGRTLISNIISLNKLLSLEIKNGGTYQIVSALNNSSVIDMSLSGDRNVHLWSNNGTLNQVWKFVYDSNRLAYQIKSLSDENLVLTWAYYSSNRDNVIVASNQNSDEQYWIPERTGAYHYFKNLINPSGALDVSGSGTTNGTNILYWSYNRATNQKFKLEEVNIPGGQAEGVLLYADANYVGKSVLLTNSVSNLRDVGMNDIASSIKFIGPYQATLYEHDNFTGAVFTPTSNVANLKDVGMNDTVSSIKITKTSGGRATGIYLYADANYVGRSVWLTSNVANLKDVGMNDTVSSVEIVGAYQATLYGDSNYTGKAYNLTHNVANLKDVGMNDIVSSIKIFSV

SEQ ID NO: 25 - DNA, 3696 bp, synthetic construct
  misc_feature 1..3696: note = A nucleotide sequence encoding TIC6547_His comprised of a histidine tag coding sequence operably linked 5' to the TIC6547 coding sequence.
  source 1..3696: mol_type = other DNA
atgcatcaccatcaccatcaccatcaccatcacggtaccgagaccgtccgcttccaatccatggatcaaaagattataaaaatgcgagaagcagtcaatgccttgttttccaataatcagttaaaattgaatattactgattacaatatagatcagattgcataccttgttgatagtatgtctgatgacgcatatcgacaagaaaaaatgaggtttctcgatcaaatcaaatttgcaaagcgcttgagtcaaaaacgcaacctgttgaattatggagattttgaaggatccaattggccaggtaagaatggatggaaaagaaataattatgtagttgtcgcatcggatcatcctatatttaaaggccgatatttacacataccaagtgcaacaaccacgatgagtggcgcaatcattccgacttatgtatatcaacgtatagatgaatcgaagttaaaaccgtatacacgttatttggtacgagggtatgttggaaagagtcaagatttagcgttacttgtttcccggtataccaaagaagtgtacaagaaaatcaatgtaccaaatgatgaggattacgatatcacatcgcatataaatagggaagagaatttatggcacaatagatatataagaggcacccaagttcaaaattcaatctctatgtgcaacaatccacatgaatttacgtgtcatattgatataggagaactggatagaaagaaaggtcctggtataaccatcggttttcaaattggaacaacagatgggatggcaacattagataatatagaagtgatagaagcacatccgttaactggatcggccttagcacgtatccaaaaacgtgaacgtaaatggaaacaaaaatggatagagaatcgaatgcaaatcgaaaaggctgtacaaacagcgcaagaggtgattcgaaatttatttacatgcccacaacaaaatcaattgaactggatgacaactcgaaacgacattacacatgcagaaacattgataaaagagattccatatcggtatagccaactttcttgtggagatttccccacactaccagaagaggcgtatgacatccttcaacaactttcaactgcagttgaaaccgcaaaagcgttatatgcacaacgaaatgtggtgaataatggggattttcaagctggattatcgaattggtatacgacagatggtgcagagatacaacaaatacagaattcgtcctctgttctagtaattaaagactgggctacaaatatttcacaggacatgcgtgtggttgaaaaaggtggctatctgctacgcgtaacagcgaaaaaagaagataccggagaaggttatataacaattagtgattgtgcagcattggtagaaaaattgacatttacaacgggggaagctgtagaaagtctggcacattctgatagtcgttcaaggctccataagcgctatgataaaaaatcagaaggatatgaaatagaatcggatcctcatttatttaatagggcgaaacaaaacggttctcttccttctagctatgtaaccaaaacgattgaaatctttccggaaaccaatcgagtacgcattgagattggagaaacaggtggaaagtttatggtggaaagtgtggaattgattcgaatggaacagatgaacgaaacaaataatccagctgtagatgttcaaactgtaatgaatgatacacctgctacacaatttgatccagtttcttttacagaatcaacggtgagtcccagaaatgctcagtatgcgtattctcatgatacaaatataggctatgaaaatcctaactggatggctgatatttcaggtgatactttatttagtgatttatctatccctggtacacataatacaatggctcttcatggaggagatattacacaatgtcaaacgatgtcactgaatacacaattacatgtaggaattcgttatttagatattcgctgtaggcatatcgataatgtttttgcgattcatcatgggcctgtgtaccaaaatacgatgtttggagatgtttgtatagccgtaagggattttttgaggaacaaccctagtgaaacagtatttatgcggataaaagaagaacatacaccagaaaataatacaagatctttttcggatacatttgcagattataagtctcaatatagcgacttattttggaattggacaggtgataacccaagattaagtgaaataagaggaaaagttgttgttttgcaaaacttttcaggggataggtttggtatctactacaatacactgaatacacaagatcaatatcatttagatacaaactgggatttatatgataaatggctatttgtaaaagagcatttgtataaagctgacgacgcttataaaagtggtggtaaacaagcatatctgaattatctaagtgggtcaggtggttcttttccttattttgttgcaagtggacatagtagtcctggtacagatgctccacaattatctacaggtctaacaacaccagcatttgcaagctggtatccggattttccacggggaagttgttttataggaatttgcacaatttactttgaaggaacaaatattcttacaagtcagtggatagagaaaaatgattttaaatatataggaatcatagctgctgattttccaggaagaacattaatttccaatattattagtttgaataaacttcttagcttagaaattaaaaatggtggtacctatcaaattgtttccgctttaaataatagtagtgttatagatatgagtctgagtggagatcgaaatgctcacctatggtccaataacggtactcctaatcaagtatggaaattcgtgtatgattcaaatagattagcataccaaattaaaagtttatccgatgaaaatttagtactaacttgggcttattatagtagtaatcgagataatgtaattgtcgcttctaatcaaaatagcgatgagcaatattggatacctgagcgcacaggcgcatatcattattttaaaaatctcatcaatccctcaggagcattagatgtaagcggatcaggaacaacaaacggaacgaatattttgtattggagttataacagagcaacgaatcaaaaattcaaactggaagaagtaaatatatctggaggtcaaactgaaggtgtacttttatatgcagaggctaattatgtagggaaatctgtactactaacaaatagtgtctccaaccttagagacgttggtatgaatgatatagctagttctataaaatttattggtccttatcaagctactctatatgaacatgatgattttacaggtgcggtttttactcccacatctaatgttgcaaatttaaaagatgttggcatgaatgatacagttagttctataaaaattacaaagacatctggaggccgagctacaggtatatatttatatgcagatgctaattatgtaggcagatctgtatggttaacatctaatgttgcaaatttaaaagatgttggcatgaatgatacagtcagttctgtagaaattgttggcgcgtatcaggccactttatatggggattccaattatacagggaaggcttataatctcactcataatgttgcaaatttaaaagatgttggcatgaatgatatagtcagttccataaaaatttttagtgtgtaa

SEQ ID NO: 26 - protein, 1231 aa, synthetic construct
  REGION 1..1231: note = The amino acid sequence of TIC6547_His.
MHHHHHHHHHHGTETVRFQSMDQKIIKMREAVNALFSNNQLKLNITDYNIDQIAYLVDSMSDDAYRQEKMRFLDQIKFAKRLSQKRNLLNYGDFEGSNWPGKNGWKRNNYVVVASDHPIFKGRYLHIPSATTTMSGAIIPTYVYQRIDESKLKPYTRYLVRGYVGKSQDLALLVSRYTKEVYKKINVPNDEDYDITSHINREENLWHNRYIRGTQVQNSISMCNNPHEFTCHIDIGELDRKKGPGITIGFQIGTTDGMATLDNIEVIEAHPLTGSALARIQKRERKWKQKWIENRMQIEKAVQTAQEVIRNLFTCPQQNQLNWMTTRNDITHAETLIKEIPYRYSQLSCGDFPTLPEEAYDILQQLSTAVETAKALYAQRNVVNNGDFQAGLSNWYTTDGAEIQQIQNSSSVLVIKDWATNISQDMRVVEKGGYLLRVTAKKEDTGEGYITISDCAALVEKLTFTTGEAVESLAHSDSRSRLHKRYDKKSEGYEIESDPHLFNRAKQNGSLPSSYVTKTIEIFPETNRVRIEIGETGGKFMVESVELIRMEQMNETNNPAVDVQTVMNDTPATQFDPVSFTESTVSPRNAQYAYSHDTNIGYENPNWMADISGDTLFSDLSIPGTHNTMALHGGDITQCQTMSLNTQLHVGIRYLDIRCRHIDNVFAIHHGPVYQNTMFGDVCIAVRDFLRNNPSETVFMRIKEEHTPENNTRSFSDTFADYKSQYSDLFWNWTGDNPRLSEIRGKVVVLQNFSGDRFGIYYNTLNTQDQYHLDTNWDLYDKWLFVKEHLYKADDAYKSGGKQAYLNYLSGSGGSFPYFVASGHSSPGTDAPQLSTGLTTPAFASWYPDFPRGSCFIGICTIYFEGTNILTSQWIEKNDFKYIGIIAADFPGRTLISNIISLNKLLSLEIKNGGTYQIVSALNNSSVIDMSLSGDRNAHLWSNNGTPNQVWKFVYDSNRLAYQIKSLSDENLVLTWAYYSSNRDNVIVASNQNSDEQYWIPERTGAYHYFKNLINPSGALDVSGSGTTNGTNILYWSYNRATNQKFKLEEVNISGGQTEGVLLYAEANYVGKSVLLTNSVSNLRDVGMNDIASSIKFIGPYQATLYEHDDFTGAVFTPTSNVANLKDVGMNDTVSSIKITKTSGGRATGIYLYADANYVGRSVWLTSNVANLKDVGMNDTVSSVEIVGAYQATLYGDSNYTGKAYNLTHNVANLKDVGMNDIVSSIKIFSV

SEQ ID NO: 27 - DNA, 3702 bp, synthetic construct
  misc_feature 1..3702: note = A nucleotide sequence encoding TIC4006_His comprised of a histidine tag coding sequence operably linked 5' to the TIC4006 coding sequence.
  source 1..3702: mol_type = other DNA
atgcatcaccatcaccatcaccatcaccatcacggtaccgagaccgtccgcttccaatccatgaatcaatatgttacaacagtgcaaaaggcagttaatgcattattttcaaataataccttacccttaaacattactgattataatatagaccagacagcatatcttgtagaacgtatatctaatgatagatattctaaagacaagatgatgttactcaatcaagtcaaatttgcgaaacgtttgagtcgagcgcgtaacttattgaaaggtggcgcttttgaattatcagataagaatagatggaagacaaacaattatgcgaatattttatcaggttctctcctatccaaaggccaatctttaaacattctaagcgcaagccctacagtaagtagtcaaattattccgactcatgtatatcaaagaatagatgaatcaaagttaaaaccatatacacgttatttagtaagagggttcgttgaaaagagtcgagatttagaactatttgtgctcagatataacaaagaggtgtataaaagaatcaatgtacccaagaatgaggattatcatatcacatcgcatttaaatgaagaagagaatccatggcacaataaatatatccaaaacactccggttcaaaattcaatctctatgcgcaagaattcacatgagtttacgtgtcatattgatataggggaactggatataaagaaaggacctggtataaccatcggttttcaaattagcacaacagatgggatggcaacattagataatatagaagtgatagaagcacatccgttaactggagacgatttaacacgtatccaaaggcgtgaacgtaaatggaaacaaaaatggctagagaatcaaatacaaatcgaaaaagctgcacaaacagcgaaagaggcgattaaaaatttatttacatgcccacaacaaaatcaattgacctggatgacaaccctaaacgacattatacaggcagaaaaattgatacaagagattccatattggtatagccgacttttaggtgaggatttccccatactaccagaagaggcatatgacacccttcaacaactttcaactgcagttgaaaccgcaaaattgttgtatgcacaacgaaatgtggtgaataatggggattttcaagctggattttcaaattggaatacgaccgatggtgcagagataaaacaaattcaggattcatcttctgttctagtaattacggactgggctgcaaatatttcacaggacatgcgtgtggttgaaaaaggtggctatctgctgcgcgtaacagcgaaaaaagaagatgccggagaaggttatataacaattagtgattgttccgtagtgatggaaaaattgacatttacaacaggggattctgtagagagtctggcacattctgatatttattcaaggatccataagcgctatgctaaaaaacaaataacaaatcatctttcagaaagatatgaaatagaatcgaatcctcatttaattaatagagcggaacaaaatgcttccctcccttctagctatgtaaccaaaacgattgaagtctttccggaaaccaatcgagtacgcgttgaaattggagaaacaggtggaacatttatcgtggaaagtgtcgaattgattcgaatggaacagatgaacgaaacaaacaatccagctgtagatattcaaactgtaatgaatgatacacccgctacacaatttgatccagtttcttttacagaatcaacggtgagtcccagaaatactcaatatgcatattctcatgattcaaatataggttatgaaaatcctaactggatggctgatatttcaggtgatactttatttagtgatttatctatccctggtacacataatacaatggctttttatggaggagatattacacaatgtcaaacgatgtcactgaatacgcaattacatgtaggaattcgttatttagatattcgctgtaggcatatcgaaaatatttttgcgattcatcatggaattgtgtaccaaaatgcgacgtttacagatgtttgtatagccgtaagagattttttgaggaacaaccctagtgagacagtatttatgcggataaaagaagaacatacagcagaaaataatacaagatcttttggggagacatttgcagactataagtctcaatatagcgacttattttggaattggacgggtgataacccaagattaagtgaaataagaggaaaagttgttgttttgcaaaatttttttggggataaatttggtatcgattacaatacactgaataaacaagatcaatatcatttaaatacaaactgggatttatatgataaatggctatttgtaaaagaacatttgtatgccgctgacgattcttataaaaatggtcgtaaacaagcatatctaaattatctaagcgggtcaggtggttcttttccttattttgttgcaagtggacacagtagtcctggtacaaatgcttcaaatctatctacagggctaacaacaccggcatttgaaagctggtatccggattttccacggggaagttgttttataggaatttgcacaatttattttgaaggaacaaatattcttacaagtgagtggatacagaaaagtgattttaaatatgtaggaatcatagctgctgattttccaggaagaacattaatttccaatattattagtctgaataatcttcttagtttagaaattaaaaatggtggtacctatcaaattgtttccgctttaaataatagtagtgttgtagatatgaatccaggagaccaaaatattcacttatggaacaataacggtactgctaatcaattatggaaattcgtatataattcaaatgaattagcataccaaattaaaagtttatctaatgaaaatttagtattaacctgggcttacaatagtagtaatccagataatgtaattgctgcttccaatcaaaataggtctgagcaatattggatacctgagcgtacgggagcatatcattattttaaaaatctaagcaatcgttcgggagcattagatgtaagcggctcagagacaaaaaacggaacaaacattctgtactggagttataaaaaagcaacaaatcaaaaattcaaactgacagaagtaaatgtatctggaggtcaagctgaaggtgtatatttatatgcagatgccaattatgtagggcaatctgtagggctaacaaatagtgtcgcagaccttagcgaagttggtatgaatgatatagctagttctataaaatttattggtccttatcaagctactctatatgagcatgctgattttaaaggtgcggtttttactcccacaactaatattgcaaatttaaaagatgttggcatgaatgatacaatcagctctataaaaattacaaagacatctggaggccgagctgcaggtatatatttatattcggatgccaattatgtgggaaggtctatatggttaacgtctaatgttgcaaatttaaaagatgttggcatgaatgatacaatcagttccgtagaaattgttggcgcatatggagtcactttatatggggatgccaattatacaggtaaggcttatgctctcacatctaatgttgcaaatttaaaagatgttggcatgaatgatatagtcagttctataaaaatttttagtgtataa

SEQ ID NO: 28 - protein, 1233 aa, synthetic construct
  REGION 1..1233: note = The amino acid sequence of TIC4006_His.
MHHHHHHHHHHGTETVRFQSMNQYVTTVQKAVNALFSNNTLPLNITDYNIDQTAYLVERISNDRYSKDKMMLLNQVKFAKRLSRARNLLKGGAFELSDKNRWKTNNYANILSGSLLSKGQSLNILSASPTVSSQIIPTHVYQRIDESKLKPYTRYLVRGFVEKSRDLELFVLRYNKEVYKRINVPKNEDYHITSHLNEEENPWHNKYIQNTPVQNSISMRKNSHEFTCHIDIGELDIKKGPGITIGFQISTTDGMATLDNIEVIEAHPLTGDDLTRIQRRERKWKQKWLENQIQIEKAAQTAKEAIKNLFTCPQQNQLTWMTTLNDIIQAEKLIQEIPYWYSRLLGEDFPILPEEAYDTLQQLSTAVETAKLLYAQRNVVNNGDFQAGFSNWNTTDGAEIKQIQDSSSVLVITDWAANISQDMRVVEKGGYLLRVTAKKEDAGEGYITISDCSVVMEKLTFTTGDSVESLAHSDIYSRIHKRYAKKQITNHLSERYEIESNPHLINRAEQNASLPSSYVTKTIEVFPETNRVRVEIGETGGTFIVESVELIRMEQMNETNNPAVDIQTVMNDTPATQFDPVSFTESTVSPRNTQYAYSHDSNIGYENPNWMADISGDTLFSDLSIPGTHNTMAFYGGDITQCQTMSLNTQLHVGIRYLDIRCRHIENIFAIHHGIVYQNATFTDVCIAVRDFLRNNPSETVFMRIKEEHTAENNTRSFGETFADYKSQYSDLFWNWTGDNPRLSEIRGKVVVLQNFFGDKFGIDYNTLNKQDQYHLNTNWDLYDKWLFVKEHLYAADDSYKNGRKQAYLNYLSGSGGSFPYFVASGHSSPGTNASNLSTGLTTPAFESWYPDFPRGSCFIGICTIYFEGTNILTSEWIQKSDFKYVGIIAADFPGRTLISNIISLNNLLSLEIKNGGTYQIVSALNNSSVVDMNPGDQNIHLWNNNGTANQLWKFVYNSNELAYQIKSLSNENLVLTWAYNSSNPDNVIAASNQNRSEQYWIPERTGAYHYFKNLSNRSGALDVSGSETKNGTNILYWSYKKATNQKFKLTEVNVSGGQAEGVYLYADANYVGQSVGLTNSVADLSEVGMNDIASSIKFIGPYQATLYEHADFKGAVFTPTTNIANLKDVGMNDTISSIKITKTSGGRAAGIYLYSDANYVGRSIWLTSNVANLKDVGMNDTISSVEIVGAYGVTLYGDANYTGKAYALTSNVANLKDVGMNDIVSSIKIFSV